SEQUENCE LISTING (330 sequences)

SEQ ID NO: 1 - protein, 5 aa, synthetic construct
EYTIH

SEQ ID NO: 2 - protein, 17 aa, synthetic construct
NINPNNGGTTYNQKFED

SEQ ID NO: 3 - protein, 6 aa, synthetic construct
GWNFDY

SEQ ID NO: 4 - protein, 115 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSS

SEQ ID NO: 5 - DNA, 15 bp, synthetic construct
  source 1..15: mol_type = other DNA
gaatacaccatccac

SEQ ID NO: 6 - DNA, 51 bp, synthetic construct
  source 1..51: mol_type = other DNA
aacattaatcctaacaatggtggtactacctacaaccagaagttcgaggac

SEQ ID NO: 7 - DNA, 18 bp, synthetic construct
  source 1..18: mol_type = other DNA
ggttggaactttgactac

SEQ ID NO: 8 - DNA, 345 bp, synthetic construct
  source 1..345: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagctggttggaactttgactactggggccaaggcaccacggtcaccgtctcctca

SEQ ID NO: 9 - protein, 30 aa, synthetic construct
EVQLVQSGAEVKKPGASVKVSCKASGYTFT

SEQ ID NO: 10 - protein, 14 aa, synthetic construct
WVRQAPGKGLEWIG

SEQ ID NO: 11 - protein, 32 aa, synthetic construct
RVTITVDKSTSTAYMELSSLRSEDTAVYYCAA

SEQ ID NO: 12 - protein, 11 aa, synthetic construct
WGQGTTVTVSS

SEQ ID NO: 13 - DNA, 90 bp, synthetic construct
  source 1..90: mol_type = other DNA
gaggtccagctggtgcagtctggagctgaggtgaagaagcctggggcctcagtgaaggtctcctgcaaggcttctggatacacattcact

SEQ ID NO: 14 - DNA, 42 bp, synthetic construct
  source 1..42: mol_type = other DNA
tgggtgaggcaggcccctggaaagggccttgagtggattgga

SEQ ID NO: 15 - DNA, 96 bp, synthetic construct
  source 1..96: mol_type = other DNA
agagtcacaatcactgtagacaagtccaccagcacagcctacatggagctcagcagcctgagatctgaggatactgcagtctattactgtgcagct

SEQ ID NO: 16 - DNA, 33 bp, synthetic construct
  source 1..33: mol_type = other DNA
tggggccaaggcaccacggtcaccgtctcctca

SEQ ID NO: 17 - protein, 5 aa, synthetic construct
EYTIH

SEQ ID NO: 18 - protein, 17 aa, synthetic construct
NINPNNGGTTYNQKFED

SEQ ID NO: 19 - protein, 6 aa, synthetic construct
YWLFDY

SEQ ID NO: 20 - protein, 115 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSS

SEQ ID NO: 21 - DNA, 15 bp, synthetic construct
  source 1..15: mol_type = other DNA
gaatacaccatccac

SEQ ID NO: 22 - DNA, 51 bp, synthetic construct; identical to SEQ ID NO: 6
  source 1..51: mol_type = other DNA

SEQ ID NO: 23 - DNA, 18 bp, synthetic construct
  source 1..18: mol_type = other DNA
tactggctgttcgactac

SEQ ID NO: 24 - DNA, 345 bp, synthetic construct
  source 1..345: mol_type = other DNA
gaggtccagctggtgcagtctggagctgaggtgaagaagcctggggcctcagtgaaggtctcctgcaaggcttctggatacacattcactgaatacaccatccactgggtgaggcaggcccctggaaagggccttgagtggattggaaacattaatcctaacaatggtggtactacctacaaccagaagttcgaggacagagtcacaatcactgtagacaagtccaccagcacagcctacatggagctcagcagcctgagatctgaggatactgcagtctattactgtgcagcttactggctgttcgactactggggccaaggcaccacggtcaccgtctcctca

SEQ ID NO: 25 - protein, 30 aa, synthetic construct; identical to SEQ ID NO: 9

SEQ ID NO: 26 - protein, 14 aa, synthetic construct
WVRQAPGKGLEWIG

SEQ ID NO: 27 - protein, 32 aa, synthetic construct; identical to SEQ ID NO: 11

SEQ ID NO: 28 - protein, 11 aa, synthetic construct
WGQGTTVTVSS

SEQ ID NO: 29 - DNA, 90 bp, synthetic construct; identical to SEQ ID NO: 13
  source 1..90: mol_type = other DNA

SEQ ID NO: 30 - DNA, 42 bp, synthetic construct; identical to SEQ ID NO: 14
  source 1..42: mol_type = other DNA

SEQ ID NO: 31 - DNA, 96 bp, synthetic construct; identical to SEQ ID NO: 15
  source 1..96: mol_type = other DNA

SEQ ID NO: 32 - DNA, 33 bp, synthetic construct; identical to SEQ ID NO: 16
  source 1..33: mol_type = other DNA

SEQ ID NO: 33 - protein, 11 aa, synthetic construct
KASQDVGTAVD

SEQ ID NO: 34 - protein, 7 aa, synthetic construct
WASTRHT

SEQ ID NO: 35 - protein, 9 aa, synthetic construct
QQYNSYPLT

SEQ ID NO: 36 - protein, 107 aa, synthetic construct
DIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIK

SEQ ID NO: 37 - DNA, 33 bp, synthetic construct
  source 1..33: mol_type = other DNA
aaggccagtcaggatgtgggtactgctgtagac

SEQ ID NO: 38 - DNA, 21 bp, synthetic construct
  source 1..21: mol_type = other DNA
tgggcatccacccggcacact

SEQ ID NO: 39 - DNA, 27 bp, synthetic construct
  source 1..27: mol_type = other DNA
cagcaatataacagctatcctctcacg

SEQ ID NO: 40 - DNA, 321 bp, synthetic construct
  source 1..321: mol_type = other DNA
gacattcagatgacccagtctcccagcaccctgtccgcatcagtaggagacagggtcaccatcacttgcaaggccagtcaggatgtgggtactgctgtagactggtatcaacagaaaccagggcaagctcctaaactactgatttactgggcatccacccggcacactggagtccctgatcgcttcagcggcagtggatctgggacagatttcactctcaccatcagcagactgcagcctgaagactttgcagtttattactgtcagcaatataacagctatcctctcacgttcggccaggggaccaaggtggatatcaaa

SEQ ID NO: 41 - protein, 23 aa, synthetic construct
DIQMTQSPSTLSASVGDRVTITC

SEQ ID NO: 42 - protein, 15 aa, synthetic construct
WYQQKPGQAPKLLIY

SEQ ID NO: 43 - protein, 32 aa, synthetic construct
GVPDRFSGSGSGTDFTLTISRLQPEDFAVYYC

SEQ ID NO: 44 - protein, 10 aa, synthetic construct
FGQGTKVDIK

SEQ ID NO: 45 - DNA, 69 bp, synthetic construct
  source 1..69: mol_type = other DNA
gacattcagatgacccagtctcccagcaccctgtccgcatcagtaggagacagggtcaccatcacttgc

SEQ ID NO: 46 - DNA, 45 bp, synthetic construct
  source 1..45: mol_type = other DNA
tggtatcaacagaaaccagggcaagctcctaaactactgatttac

SEQ ID NO: 47 - DNA, 96 bp, synthetic construct
  source 1..96: mol_type = other DNA
ggagtccctgatcgcttcagcggcagtggatctgggacagatttcactctcaccatcagcagactgcagcctgaagactttgcagtttattactgt

SEQ ID NO: 48 - DNA, 30 bp, synthetic construct
  source 1..30: mol_type = other DNA
ttcggccaggggaccaaggtggatatcaaa

SEQ ID NO: 49 - protein, 5 aa, synthetic construct
NYYMS

SEQ ID NO: 50 - protein, 17 aa, synthetic construct
AINSDGGITYYLDTVKG

SEQ ID NO: 51 - protein, 10 aa, synthetic construct
HRSGYFSMDY

SEQ ID NO: 52 - protein, 119 aa, synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSS

SEQ ID NO: 53 - DNA, 15 bp, synthetic construct
  source 1..15: mol_type = other DNA
aactattacatgtct

SEQ ID NO: 54 - DNA, 51 bp, synthetic construct
  source 1..51: mol_type = other DNA
gccattaatagtgatggtggtatcacctactatctagacactgtgaagggc

SEQ ID NO: 55 - DNA, 30 bp, synthetic construct
  source 1..30: mol_type = other DNA
caccgctcgggctacttttctatggactac

SEQ ID NO: 56 - DNA, 357 bp, synthetic construct
  source 1..357: mol_type = other DNA
gacgtgaagctcgtggagtctgggggaggcttagtgaagcttggaggatccctgaaactctcctgtgcagcctctggattcactttcagtaactattacatgtcttgggttcgccagactccagagaagaggctggagttggtcgcagccattaatagtgatggtggtatcacctactatctagacactgtgaagggccgattcaccatttcaagagacaatgccaagaacaccctgtacctgcaaatgagcagtctgaagtctgaggacacagccttgttttactgtgcaagacaccgctcgggctacttttctatggactactggggtcaaggaacctcagtcaccgtctcctca

SEQ ID NO: 57 - protein, 30 aa, synthetic construct
DVKLVESGGGLVKLGGSLKLSCAASGFTFS

SEQ ID NO: 58 - protein, 14 aa, synthetic construct
WVRQTPEKRLELVA

SEQ ID NO: 59 - protein, 32 aa, synthetic construct
RFTISRDNAKNTLYLQMSSLKSEDTALFYCAR

SEQ ID NO: 60 - protein, 11 aa, synthetic construct
WGQGTSVTVSS

SEQ ID NO: 61 - DNA, 90 bp, synthetic construct
  source 1..90: mol_type = other DNA
gacgtgaagctcgtggagtctgggggaggcttagtgaagcttggaggatccctgaaactctcctgtgcagcctctggattcactttcagt

SEQ ID NO: 62 - DNA, 42 bp, synthetic construct
  source 1..42: mol_type = other DNA
tgggttcgccagactccagagaagaggctggagttggtcgca

SEQ ID NO: 63 - DNA, 96 bp, synthetic construct
  source 1..96: mol_type = other DNA
cgattcaccatttcaagagacaatgccaagaacaccctgtacctgcaaatgagcagtctgaagtctgaggacacagccttgttttactgtgcaaga

SEQ ID NO: 64 - DNA, 33 bp, synthetic construct
  source 1..33: mol_type = other DNA
tggggtcaaggaacctcagtcaccgtctcctca

SEQ ID NO: 65 - protein, 5 aa, synthetic construct
NYYMS

SEQ ID NO: 66 - protein, 17 aa, synthetic construct
AINSDGGITYYLDTVKG

SEQ ID NO: 67 - protein, 10 aa, synthetic construct
HRSGYFSMDY

SEQ ID NO: 68 - protein, 119 aa, synthetic construct
DVKLVESGGGLVKPGGSLRLSCAASGFTFSNYYMSWVRQAPGKGLELVAAINSDGGITYYLDTVKGRFTISRDNAKNTLYLQMSSLKSEDTALYYCARHRSGYFSMDYWGQGTSVTVSS

SEQ ID NO: 69 - DNA, 15 bp, synthetic construct
  source 1..15: mol_type = other DNA
aactactacatgagc

SEQ ID NO: 70 - DNA, 51 bp, synthetic construct
  source 1..51: mol_type = other DNA
gccattaacagtgacggtggcatcacctactacctggacaccgtgaagggc

SEQ ID NO: 71 - DNA, 30 bp, synthetic construct
  source 1..30: mol_type = other DNA
cacaggagcggctacttctctatggactac

SEQ ID NO: 72 - DNA, 357 bp, synthetic construct
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgagcagcctgaagagcgaggacacggccctgtattactgtgcgagacacaggagcggctacttctctatggactactggggccagggcaccagcgtcactgtctcctca

SEQ ID NO: 73 - protein, 30 aa, synthetic construct
DVKLVESGGGLVKPGGSLRLSCAASGFTFS

SEQ ID NO: 74 - protein, 14 aa, synthetic construct
WVRQAPGKGLELVA

SEQ ID NO: 75 - protein, 32 aa, synthetic construct
RFTISRDNAKNTLYLQMSSLKSEDTALYYCAR

SEQ ID NO: 76 - protein, 11 aa, synthetic construct
WGQGTSVTVSS

SEQ ID NO: 77 - DNA, 90 bp, synthetic construct
  source 1..90: mol_type = other DNA
gacgtgaagctggtggagtctgggggaggcttggtcaagcctggaggatccctgagactctcctgtgcagcctctggattcaccttcagt

SEQ ID NO: 78 - DNA, 42 bp, synthetic construct
  source 1..42: mol_type = other DNA
tgggtgcgccaggctccagggaaggggctggagctggttgcc

SEQ ID NO: 79 - DNA, 96 bp, synthetic construct
  source 1..96: mol_type = other DNA
cgattcaccatctccagggacaacgccaagaacaccctgtatctgcaaatgagcagcctgaagagcgaggacacggccctgtattactgtgcgaga

SEQ ID NO: 80 - DNA, 33 bp, synthetic construct
  source 1..33: mol_type = other DNA
tggggccagggcaccagcgtcactgtctcctca

SEQ ID NO: 81 - protein, 5 aa, synthetic construct
NYYMS

SEQ ID NO: 82 - protein, 17 aa, synthetic construct
AINSDGGITYYLDTVKG

SEQ ID NO: 83 - protein, 10 aa, synthetic construct
HRSGYFSMDY

SEQ ID NO: 84 - protein, 119 aa, synthetic construct
DVKLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 85 - DNA, 15 bp, synthetic construct
  source 1..15: mol_type = other DNA
aactactacatgagc

SEQ ID NO: 86 - DNA, 50 bp, synthetic construct
  source 1..50: mol_type = other DNA
ccattaacagtgacggtggcatcacctactacctggacaccgtgaagggc

SEQ ID NO: 87 - DNA, 30 bp, synthetic construct; identical to SEQ ID NO: 71
  source 1..30: mol_type = other DNA

SEQ ID NO: 88 - DNA, 357 bp, synthetic construct
  source 1..357: mol_type = other DNA
gacgtgaagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 89 - protein, 30 aa, synthetic construct; identical to SEQ ID NO: 73

SEQ ID NO: 90 - protein, 14 aa, synthetic construct
WVRQAPGKGLELVA

SEQ ID NO: 91 - protein, 32 aa, synthetic construct
RFTISRDNAKNTLYLQMNSLRAEDTALYYCAR

SEQ ID NO: 92 - protein, 11 aa, synthetic construct
WGQGTLVTVSS

SEQ ID NO: 93 - DNA, 90 bp, synthetic construct; identical to SEQ ID NO: 77
  source 1..90: mol_type = other DNA

SEQ ID NO: 94 - DNA, 42 bp, synthetic construct; identical to SEQ ID NO: 78
  source 1..42: mol_type = other DNA

SEQ ID NO: 95 - DNA, 96 bp, synthetic construct
  source 1..96: mol_type = other DNA
cgattcaccatctccagggacaacgccaagaacaccctgtatctgcaaatgaacagcctgagggccgaggacacggccctgtattactgtgcgaga

SEQ ID NO: 96 - DNA, 33 bp, synthetic construct
  source 1..33: mol_type = other DNA
tggggccagggcaccctggtcactgtctcctca

SEQ ID NO: 97 - protein, 5 aa, synthetic construct
NYYMS

SEQ ID NO: 98 - protein, 17 aa, synthetic construct
AINSDGGITYYLDTVKG

SEQ ID NO: 99 - protein, 10 aa, synthetic construct
HRSGYFSMDY

SEQ ID NO: 100 - protein, 119 aa, synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSS

SEQ ID NO: 101 - DNA, 15 bp, synthetic construct
  source 1..15: mol_type = other DNA
aactactacatgagc

SEQ ID NO: 102 - DNA, 51 bp, synthetic construct; identical to SEQ ID NO: 70
  source 1..51: mol_type = other DNA

SEQ ID NO: 103 - DNA, 30 bp, synthetic construct; identical to SEQ ID NO: 71
  source 1..30: mol_type = other DNA

SEQ ID NO: 104 - DNA, 357 bp, synthetic construct
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagctggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 105 - protein, 30 aa, synthetic construct
EVQLVESGGGLVKPGGSLRLSCAASGFTFS

SEQ ID NO: 106 - protein, 14 aa, synthetic construct
WVRQAPGKGLELVA

SEQ ID NO: 107 - protein, 32 aa, synthetic construct; identical to SEQ ID NO: 91

SEQ ID NO: 108 - protein, 11 aa, synthetic construct
WGQGTLVTVSS

SEQ ID NO: 109 - DNA, 90 bp, synthetic construct
  source 1..90: mol_type = other DNA
gaggtgcagctggtggagtctgggggaggcttggtcaagcctggaggatccctgagactctcctgtgcagcctctggattcaccttcagt

SEQ ID NO: 110 - DNA, 42 bp, synthetic construct; identical to SEQ ID NO: 78
  source 1..42: mol_type = other DNA

SEQ ID NO: 111 - DNA, 96 bp, synthetic construct; identical to SEQ ID NO: 95
  source 1..96: mol_type = other DNA

SEQ ID NO: 112 - DNA, 33 bp, synthetic construct; identical to SEQ ID NO: 96
  source 1..33: mol_type = other DNA

SEQ ID NO: 113 - protein, 5 aa, synthetic construct
NYYMS

SEQ ID NO: 114 - protein, 17 aa, synthetic construct
AINSDGGITYYLDTVKG

SEQ ID NO: 115 - protein, 10 aa, synthetic construct
HRSGYFSMDY

SEQ ID NO: 116 - protein, 119 aa, synthetic construct
EVQLVESGGGLVKPGGSLRLSCAASGFTFSNYYMSWVRQAPGKGLEWVAAINSDGGITYYLDTVKGRFTISRDNAKNTLYLQMNSLRAEDTALYYCARHRSGYFSMDYWGQGTLVTVSS

SEQ ID NO: 117 - DNA, 15 bp, synthetic construct
  source 1..15: mol_type = other DNA
aactactacatgagc

SEQ ID NO: 118 - DNA, 51 bp, synthetic construct; identical to SEQ ID NO: 70
  source 1..51: mol_type = other DNA

SEQ ID NO: 119 - DNA, 30 bp, synthetic construct; identical to SEQ ID NO: 71
  source 1..30: mol_type = other DNA

SEQ ID NO: 120 - DNA, 357 bp, synthetic construct
  source 1..357: mol_type = other DNA
gaggtgcagctggtggagtctgggggaggcttggtcaagcctggaggatccctgagactctcctgtgcagcctctggattcaccttcagtaactactacatgagctgggtgcgccaggctccagggaaggggctggagtgggttgccgccattaacagtgacggtggcatcacctactacctggacaccgtgaagggccgattcaccatctccagggacaacgccaagaacaccctgtatctgcaaatgaacagcctgagggccgaggacacggccctgtattactgtgcgagacacaggagcggctacttctctatggactactggggccagggcaccctggtcactgtctcctca

SEQ ID NO: 121 - protein, 30 aa, synthetic construct; identical to SEQ ID NO: 105

SEQ ID NO: 122 - protein, 14 aa, synthetic construct
WVRQAPGKGLEWVA

SEQ ID NO: 123 - protein, 32 aa, synthetic construct; identical to SEQ ID NO: 91

SEQ ID NO: 124 - protein, 11 aa, synthetic construct
WGQGTLVTVSS

SEQ ID NO: 125 - DNA, 90 bp, synthetic construct; identical to SEQ ID NO: 109
  source 1..90: mol_type = other DNA

SEQ ID NO: 126 - DNA, 42 bp, synthetic construct
  source 1..42: mol_type = other DNA
tgggtgcgccaggctccagggaaggggctggagtgggttgcc

SEQ ID NO: 127 - DNA, 96 bp, synthetic construct; identical to SEQ ID NO: 95
  source 1..96: mol_type = other DNA

SEQ ID NO: 128 - DNA, 33 bp, synthetic construct; identical to SEQ ID NO: 96
  source 1..33: mol_type = other DNA

SEQ ID NO: 129 - protein, 11 aa, synthetic construct
KASQNVVSAVA

SEQ ID NO: 130 - protein, 7 aa, synthetic construct
SASNRYT

SEQ ID NO: 131 - protein, 9 aa, synthetic construct
QQYSNYPWT

SEQ ID NO: 132 - protein, 107 aa, synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIK

SEQ ID NO: 133 - DNA, 33 bp, synthetic construct
  source 1..33: mol_type = other DNA
aaggccagtcagaatgtggtttctgctgttgcc

SEQ ID NO: 134 - DNA, 21 bp, synthetic construct
  source 1..21: mol_type = other DNA
tcagcatccaatcggtacact

SEQ ID NO: 135 - DNA, 27 bp, synthetic construct
  source 1..27: mol_type = other DNA
caacaatatagcaactatccgtggacg

SEQ ID NO: 136 - DNA, 321 bp, synthetic construct
  source 1..321: mol_type = other DNA
gacattgtgatgacccagtctcaaagattcatgtccacaacagtaggagacagggtcagcatcacctgcaaggccagtcagaatgtggtttctgctgttgcctggtatcaacagaaaccaggacaatctcctaaactactgatttactcagcatccaatcggtacactggagtccctgatcgcttcacaggcagtggatctgggacagatttcactctcaccattagcaatatgcagtctgaagacctggctgattttttctgtcaacaatatagcaactatccgtggacgttcggtggaggcaccaagctggaaatcaaa

SEQ ID NO: 137 - protein, 23 aa, synthetic construct
DIVMTQSQRFMSTTVGDRVSITC

SEQ ID NO: 138 - protein, 15 aa, synthetic construct
WYQQKPGQSPKLLIY

SEQ ID NO: 139 - protein, 32 aa, synthetic construct
GVPDRFTGSGSGTDFTLTISNMQSEDLADFFC

SEQ ID NO: 140 - protein, 10 aa, synthetic construct
FGGGTKLEIK

SEQ ID NO: 141 - DNA, 69 bp, synthetic construct
  source 1..69: mol_type = other DNA
gacattgtgatgacccagtctcaaagattcatgtccacaacagtaggagacagggtcagcatcacctgc

SEQ ID NO: 142 - DNA, 45 bp, synthetic construct
  source 1..45: mol_type = other DNA
tggtatcaacagaaaccaggacaatctcctaaactactgatttac

SEQ ID NO: 143 - DNA, 96 bp, synthetic construct
  source 1..96: mol_type = other DNA
ggagtccctgatcgcttcacaggcagtggatctgggacagatttcactctcaccattagcaatatgcagtctgaagacctggctgattttttctgt

SEQ ID NO: 144 - DNA, 30 bp, synthetic construct
  source 1..30: mol_type = other DNA
ttcggtggaggcaccaagctggaaatcaaa

SEQ ID NO: 145 - protein, 11 aa, synthetic construct
KASQNVVSAVA

SEQ ID NO: 146 - protein, 7 aa, synthetic construct
SASNRYT

SEQ ID NO: 147 - protein, 9 aa, synthetic construct
QQYSNYPWT

SEQ ID NO: 148 - protein, 107 aa, synthetic construct
DIVMTQSPSFLSASVGDRVTITCKASQNVVSAVAWYQQKPGQAPKLLIYSASNRYTGVPDRFTGSGSGTDFTLTISSLQSEDLADYFCQQYSNYPWTFGGGTKVEIK

SEQ ID NO: 149 - DNA, 33 bp, synthetic construct
  source 1..33: mol_type = other DNA
aaggcaagtcagaacgtggtgagtgctgtggcc

SEQ ID NO: 150 - DNA, 21 bp, synthetic construct
  source 1..21: mol_type = other DNA
agcgcctccaacaggtacacc

SEQ ID NO: 151 - DNA, 27 bp, synthetic construct
  source 1..27: mol_type = other DNA
caacagtacagcaattacccttggacg

SEQ ID NO: 152 - DNA, 321 bp, synthetic construct
  source 1..321: mol_type = other DNA
gacatcgtgatgacccagtctccatccttcctgtctgcatctgtaggagacagagtcaccatcacttgcaaggcaagtcagaacgtggtgagtgctgtggcctggtatcagcagaaaccagggcaggctcctaagctcctgatctatagcgcctccaacaggtacaccggggtcccagacaggttcaccggcagtggatctgggacagatttcactctcaccatcagcagcctgcagagcgaagatctggcagactatttctgtcaacagtacagcaattacccttggacgttcggcggcgggaccaaggtggaaatcaaa

SEQ ID NO: 153 - protein, 23 aa, synthetic construct
DIVMTQSPSFLSASVGDRVTITC

SEQ ID NO: 154 - protein, 15 aa, synthetic construct
WYQQKPGQAPKLLIY

SEQ ID NO: 155 - protein, 32 aa, synthetic construct
GVPDRFTGSGSGTDFTLTISSLQSEDLADYFC

SEQ ID NO: 156 - protein, 10 aa, synthetic construct
FGGGTKVEIK

SEQ ID NO: 157 - DNA, 69 bp, synthetic construct
  source 1..69: mol_type = other DNA
gacatcgtgatgacccagtctccatccttcctgtctgcatctgtaggagacagagtcaccatcacttgc

SEQ ID NO: 158 - DNA, 45 bp, synthetic construct
  source 1..45: mol_type = other DNA
tggtatcagcagaaaccagggcaggctcctaagctcctgatctat

SEQ ID NO: 159 - DNA, 96 bp, synthetic construct
  source 1..96: mol_type = other DNA
ggggtcccagacaggttcaccggcagtggatctgggacagatttcactctcaccatcagcagcctgcagagcgaagatctggcagactatttctgt

SEQ ID NO: 160 - DNA, 30 bp, synthetic construct
  source 1..30: mol_type = other DNA
ttcggcggcgggaccaaggtggaaatcaaa

SEQ ID NO: 161 - protein, 11 aa, synthetic construct
KASQNVVSAVA

SEQ ID NO: 162 - protein, 7 aa, synthetic construct
SASNRYT

SEQ ID NO: 163 - protein, 9 aa, synthetic construct
QQYSNYPWT

SEQ ID NO: 164 - protein, 107 aa, synthetic construct
DIVMTQSPSSLSASVGDRVTITCKASQNVVSAVAWYQQKPGQAPRLLIYSASNRYTGVPDRFTGSGSGTDFTLTISSLQAEDLADYFCQQYSNYPWTFGGGTKVEIK

SEQ ID NO: 165 - DNA, 33 bp, synthetic construct; identical to SEQ ID NO: 149
  source 1..33: mol_type = other DNA

SEQ ID NO: 166 - DNA, 21 bp, synthetic construct
  source 1..21: mol_type = other DNA
agcgcctccaacaggtacacc

SEQ ID NO: 167 - DNA, 27 bp, synthetic construct
  source 1..27: mol_type = other DNA
caacagtacagcaattacccttggacg

SEQ ID NO: 168 - DNA, 321 bp, synthetic construct
  source 1..321: mol_type = other DNA
gacatcgtgatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcaccggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 169 - protein, 23 aa, synthetic construct
DIVMTQSPSSLSASVGDRVTITC

SEQ ID NO: 170 - protein, 15 aa, synthetic construct
WYQQKPGQAPRLLIY

SEQ ID NO: 171 - protein, 32 aa, synthetic construct
GVPDRFTGSGSGTDFTLTISSLQAEDLADYFC

SEQ ID NO: 172 - protein, 10 aa, synthetic construct
FGGGTKVEIK

SEQ ID NO: 173 - DNA, 69 bp, synthetic construct
  source 1..69: mol_type = other DNA
gacatcgtgatgacccagtctccatccagcctgtctgcatctgtaggagacagagtcaccatcacttgc

SEQ ID NO: 174 - DNA, 45 bp, synthetic construct
  source 1..45: mol_type = other DNA
tggtatcagcagaaaccagggcaggctcctaggctcctgatctat

SEQ ID NO: 175 - DNA, 96 bp, synthetic construct
  source 1..96: mol_type = other DNA
ggggtcccagacaggttcaccggcagtggatctgggacagatttcactctcaccatcagcagcctgcaggccgaagatctggcagactatttctgt

SEQ ID NO: 176 - DNA, 30 bp, synthetic construct; identical to SEQ ID NO: 160
  source 1..30: mol_type = other DNA

SEQ ID NO: 177 - protein, 11 aa, synthetic construct
KASQNVVSAVA

SEQ ID NO: 178 - protein, 7 aa, synthetic construct
SASNRYT

SEQ ID NO: 179 - protein, 9 aa, synthetic construct
QQYSNYPWT

SEQ ID NO: 180 - protein, 107 aa, synthetic construct
DIQMTQSPSSLSASVGDRVTITCKASQNVVSAVAWYQQKPGQAPRLLIYSASNRYTGVPDRFSGSGSGTDFTLTISSLQAEDLADYFCQQYSNYPWTFGGGTKVEIK

SEQ ID NO: 181 - DNA, 33 bp, synthetic construct; identical to SEQ ID NO: 149
  source 1..33: mol_type = other DNA

SEQ ID NO: 182 - DNA, 21 bp, synthetic construct
  source 1..21: mol_type = other DNA
agcgcctccaacaggtacacc

SEQ ID NO: 183 - DNA, 27 bp, synthetic construct
  source 1..27: mol_type = other DNA
caacagtacagcaattacccttggacg

SEQ ID NO: 184 - DNA, 321 bp, synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactatttctgtcaacagtacagcaattacccttggacgttcggcggcgggaccaaggtggaaatcaaa

SEQ ID NO: 185 - protein, 23 aa, synthetic construct
DIQMTQSPSSLSASVGDRVTITC

SEQ ID NO: 186 - protein, 15 aa, synthetic construct
WYQQKPGQAPRLLIY

SEQ ID NO: 187 - protein, 32 aa, synthetic construct
GVPDRFSGSGSGTDFTLTISSLQAEDLADYFC

SEQ ID NO: 188 - protein, 10 aa, synthetic construct
FGGGTKVEIK

SEQ ID NO: 189 - DNA, 69 bp, synthetic construct
  source 1..69: mol_type = other DNA
gacatccagatgacccagtctccatccagcctgtctgcatctgtaggagacagagtcaccatcacttgc

SEQ ID NO: 190 - DNA, 45 bp, synthetic construct; identical to SEQ ID NO: 174
  source 1..45: mol_type = other DNA

SEQ ID NO: 191 - DNA, 96 bp, synthetic construct
  source 1..96: mol_type = other DNA
ggggtcccagacaggttcagcggcagtggatctgggacagatttcactctcaccatcagcagcctgcaggccgaagatctggcagactatttctgt

SEQ ID NO: 192 - DNA, 30 bp, synthetic construct; identical to SEQ ID NO: 160
  source 1..30: mol_type = other DNA

SEQ ID NO: 193 - protein, 11 aa, synthetic construct
KASQNVVSAVA

SEQ ID NO: 194 - protein, 7 aa, synthetic construct
SASNRYT

SEQ ID NO: 195 - protein, 9 aa, synthetic construct
QQYSNYPWT

SEQ ID NO: 196 - protein, 107 aa, synthetic construct
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIK

SEQ ID NO: 197 - DNA, 33 bp, synthetic construct; identical to SEQ ID NO: 149
  source 1..33: mol_type = other DNA

SEQ ID NO: 198 - DNA, 21 bp, synthetic construct
  source 1..21: mol_type = other DNA
agcgcctccaacaggtacacc

SEQ ID NO: 199 - DNA, 27 bp, synthetic construct
  source 1..27: mol_type = other DNA
caacagtacagcaattacccttggacg

SEQ ID NO: 200 - DNA, 321 bp, synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggctcctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 201 - protein, 23 aa, synthetic construct
DIQMTQSPSSLSASVGDRVTITC

SEQ ID NO: 202 - protein, 15 aa, synthetic construct
WYQQKPGQAPRLLIY

SEQ ID NO: 203 - protein, 32 aa, synthetic construct
GVPDRFSGSGSGTDFTLTISSLQAEDLADYYC

SEQ ID NO: 204 - protein, 10 aa, synthetic construct
FGGGTKVEIK

SEQ ID NO: 205 - DNA, 69 bp, synthetic construct; identical to SEQ ID NO: 189
  source 1..69: mol_type = other DNA

SEQ ID NO: 206 - DNA, 45 bp, synthetic construct; identical to SEQ ID NO: 174
  source 1..45: mol_type = other DNA

SEQ ID NO: 207 - DNA, 96 bp, synthetic construct
  source 1..96: mol_type = other DNA
ggggtcccagacaggttcagcggcagtggatctgggacagatttcactctcaccatcagcagcctgcaggccgaagatctggcagactattactgt

SEQ ID NO: 208 - DNA, 30 bp, synthetic construct; identical to SEQ ID NO: 160
  source 1..30: mol_type = other DNA

SEQ ID NO: 209 - protein, 11 aa, synthetic construct
KASQNVVSAVA

SEQ ID NO: 210 - protein, 7 aa, synthetic construct
SASNRYT

SEQ ID NO: 211 - protein, 9 aa, synthetic construct
QQYSNYPWT

SEQ ID NO: 212 - protein, 107 aa, synthetic construct
DIQMTQSPSSLSASVGDRVTITCKASQNVVSAVAWYQQKPGQAPRRLIYSASNRYTGVPDRFSGSGSGTDFTLTISSLQAEDLADYYCQQYSNYPWTFGGGTKVEIK

SEQ ID NO: 213 - DNA, 33 bp, synthetic construct; identical to SEQ ID NO: 149
  source 1..33: mol_type = other DNA

SEQ ID NO: 214 - DNA, 21 bp, synthetic construct
  source 1..21: mol_type = other DNA
agcgcctccaacaggtacacc

SEQ ID NO: 215 - DNA, 27 bp, synthetic construct
  source 1..27: mol_type = other DNA
caacagtacagcaattacccttggacg

SEQ ID NO: 216 - DNA, 321 bp, synthetic construct
  source 1..321: mol_type = other DNA
gacatccagatgacccagtctccatccagcctgtctgcatctgtaggagacagagtcaccatcacttgcaaggcaagtcagaacgtggtgagtgctgtggcctggtatcagcagaaaccagggcaggctcctaggaggctgatctatagcgcctccaacaggtacaccggggtcccagacaggttcagcggcagtggatctgggacagatttcactctcaccatcagcagcctgcaggccgaagatctggcagactattactgtcaacagtacagcaattacccttggacgttcggcggcgggaccaaggtggaaatcaaa

SEQ ID NO: 217 - protein, 23 aa, synthetic construct
DIQMTQSPSSLSASVGDRVTITC

SEQ ID NO: 218 - protein, 15 aa, synthetic construct
WYQQKPGQAPRRLIY

SEQ ID NO: 219 - protein, 32 aa, synthetic construct; identical to SEQ ID NO: 203

SEQ ID NO: 220 - protein, 10 aa, synthetic construct
FGGGTKVEIK

SEQ ID NO: 221 - DNA, 69 bp, synthetic construct; identical to SEQ ID NO: 189
  source 1..69: mol_type = other DNA

SEQ ID NO: 222 - DNA, 45 bp, synthetic construct
  source 1..45: mol_type = other DNA
tggtatcagcagaaaccagggcaggctcctaggaggctgatctat

SEQ ID NO: 223 - DNA, 96 bp, synthetic construct; identical to SEQ ID NO: 207
  source 1..96: mol_type = other DNA

SEQ ID NO: 224 - DNA, 30 bp, synthetic construct; identical to SEQ ID NO: 160
  source 1..30: mol_type = other DNA

SEQ ID NO: 225 - protein, 330 aa, synthetic construct [sequence omitted]

SEQ ID NO: 226 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 227 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 228 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 229 - protein, 107 aa, synthetic construct [sequence omitted]

SEQ ID NO: 230 - protein, 449 aa, synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 231 - protein, 449 aa, synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 232 - protein, 446 aa, synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 233 - protein, 446 aa, synthetic construct
EVQLVESGGGLVKPGGSLRLSCAASGFTFSNYYMSWVRQAPGKGLELVAAINSDGGITYYLDTVKGRFTISRDNAKNTLYLQMNSLRAEDTALYYCARHRSGYFSMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 234 - protein, 214 aa, synthetic construct
DIQMTQSPSSLSASVGDRVTITCKASQNVVSAVAWYQQKPGQAPRLLIYSASNRYTGVPDRFSGSGSGTDFTLTISSLQAEDLADYYCQQYSNYPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 235 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 236 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 237 - protein, 327 aa, synthetic construct; identical to SEQ ID NO: 228

SEQ ID NO: 238 - protein, 107 aa, synthetic construct [sequence omitted]

SEQ ID NO: 239 - protein, 445 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 240 - protein, 442 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAGWNFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 241 - protein, 445 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLAQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNQYTQKSLSLSPGK

SEQ ID NO: 242 - protein, 442 aa, synthetic construct
EVQLVQSGAEVKKPGASVKVSCKASGYTFTEYTIHWVRQAPGKGLEWIGNINPNNGGTTYNQKFEDRVTITVDKSTSTAYMELSSLRSEDTAVYYCAAYWLFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLAQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNQYTQKSLSLSLGK

SEQ ID NO: 243 - protein, 214 aa, synthetic construct
DIQMTQSPSTLSASVGDRVTITCKASQDVGTAVDWYQQKPGQAPKLLIYWASTRHTGVPDRFSGSGSGTDFTLTISRLQPEDFAVYYCQQYNSYPLTFGQGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 244 - protein, 115 aa, synthetic construct
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSS

SEQ ID NO: 245 - protein, 107 aa, synthetic construct
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIK

SEQ ID NO: 246 - protein, 93 aa, Homo sapiens
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 247 - protein, 154 aa, Homo sapiens
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 248 - protein, 143 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 249 - protein, 136 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 250 - protein, 129 aa, Homo sapiens
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 251 - protein, 114 aa, Homo sapiens
TDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 252 - protein, 104 aa, Homo sapiens
QLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEE

SEQ ID NO: 253 - protein, 851 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNAEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 254 - protein, 850 aa, Homo sapiens
MTSHYVIAIFALMSSCLATAGPEPGALCELSPVSASHPVQALMESFTVLSGCASRGTTGLPQEVHVLNLRTAGQGPGQLQREVTLHLNPISSVHIHHKSVVFLLNSPHPLVWHLKTERLATGVSRLFLVSEGSVVQFSSANFSLTAETEERNFPHGNEHLLNWARKEYGAVTSFTELKIARNIYIKVGEDQVFPPKCNIGKNFLSLNYLAEYLQPKAAEGCVMSSQPQNEEVHIIELITPNSNPYSAFQVDITIDIRPSQEDLEVVKNLILILKCKKSVNWVIKSFDVKGSLKIIAPNSIGFGKESERSMTMTKSIRDDIPSTQGNLVKWALDNGYSPITSYTMAPVANRFHLRLENNEEMGDEEVHTIPPELRILLDPGALPALQNPPIRGGEGQNGGLPFPFPDISRRVWNEEGEDGLPRPKDPVIPSIQLFPGLREPEEVQGSVDIALSVKCDNEKMIVAVEKDSFQASGYSGMDVTLLDPTCKAKMNGTHFVLESPLNGCGTRPRWSALDGVVYYNSIVIQVPALGDSSGWPDGYEDLESGDNGFPGDMDEGDASLFTRPEIVVFNCSLQQVRNPSSFQEQPHGNITFNMELYNTDLFLVPSQGVFSVPENGHVYVEVSVTKAEQELGFAIQTCFISPYSNPDRMSHYTIIENICPKDESVKFYSPKRVHFPIPQADMDKKRFSFVFKPVFNTSLLFLQCELTLCTKMEKHPQKLPKCVPPDEACTSLDASIIWAMMQNKKTFTKPLAVIHHEAESKEKGPSMKEPNPISPPIFHGLDTLTVMGIAFAAFVIGALLTGALWYIYSHTGETAGRQQVPTSPPASENSSAAHSIGSTQSTPCSSSSTA

SEQ ID NO: 255 - protein, 620 aa, synthetic construct
  VARIANT 468: note = K or A
MGWSCIILFLVATATGVHSDVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGXGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 256 - protein, 233 aa, synthetic construct
MGWSCIILFLVATATGVHSDIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 257 - protein, 601 aa, synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 258 - protein, 601 aa, synthetic construct
DVKLVESGGGLVKLGGSLKLSCAASGFTFSNYYMSWVRQTPEKRLELVAAINSDGGITYYLDTVKGRFTISRDNAKNTLYLQMSSLKSEDTALFYCARHRSGYFSMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 259 - protein, 214 aa, synthetic construct
DIVMTQSQRFMSTTVGDRVSITCKASQNVVSAVAWYQQKPGQSPKLLIYSASNRYTGVPDRFTGSGSGTDFTLTISNMQSEDLADFFCQQYSNYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 616 aa, synthetic construct
  VARIANT 468: note = K or A
MGWSCIILFLVATATGVHSEVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGXGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 261 - protein, 233 aa, synthetic construct
MGWSCIILFLVATATGVHSDIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 597 aa, synthetic construct
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 263 - protein, 597 aa, synthetic construct
EVQLVQSGPEVKKPGATVKISCKTSGYTFTEYTIHWVKQAPGKGLEWIGNINPNNGGTTYNQKFEDKATLTVDKSTDTAYMELSSLRSEDTAVYYCAAGWNFDYWGQGTLLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGAGGGGSGGGGSGGGGSTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 264 - protein, 214 aa, synthetic construct
DIQMTQSPSSLSTSVGDRVTLTCKASQDVGTAVDWYQQKPGPSPKLLIYWASTRHTGIPSRFSGSGSGTDFTLTISSLQPEDFADYYCQQYNSYPLTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 265 - protein, 127 aa, synthetic construct
QLQLQESGPGLVKPSETLSLTCTVSGGSINSSSYYWGWLRQSPGKGLEWIGSFFYTGSTYYNPSLRSRLTISVDTSKNQFSLMLSSVTAADTAVYYCARQSTYYYGSGNYYGWFDRWDQGTLVTVSS

SEQ ID NO: 266 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGQGTKVEIK

SEQ ID NO: 267 - protein, 11 aa, synthetic construct
GGSINGSSSYY

SEQ ID NO: 268 - protein, 7 aa, synthetic construct
FFYTGST

SEQ ID NO: 269 - protein, 19 aa, synthetic construct
ARQSTYYYGSGNYYGWFDR

SEQ ID NO: 270 - protein, 6 aa, synthetic construct
QSVSSY

SEQ ID NO: 271 - skipped

SEQ ID NO: 272 - protein, 9 aa, synthetic construct
QQRSNWPPA

SEQ ID NO: 273 - protein, 25 aa, synthetic construct
QLQLQESGPGLVKPSETLSLTCTVS

SEQ ID NO: 274 - protein, 17 aa, synthetic construct
WGWLRQSPGKGLEWIGS

SEQ ID NO: 275 - protein, 37 aa, synthetic construct
YYNPSLRSRLTISVDTSKNQFSLMLSSVTAADTAVYY

SEQ ID NO: 276 - protein, 11 aa, synthetic construct
WDQGTLVTVSS

SEQ ID NO: 277 - protein, 26 aa, synthetic construct [sequence omitted]

SEQ ID NO: 278 - protein, 17 aa, synthetic construct
LAWYQQKPGQAPRLLIY

SEQ ID NO: 279 - protein, 35 aa, synthetic construct
NRATGIPARFSGSGSGTDFTLTISSLEPEDFAVYY

SEQ ID NO: 280-299 - skipped (20 entries)

SEQ ID NO: 300 - protein, 10 aa, synthetic construct
FGQGTKVEIK

SEQ ID NO: 301 - protein, 330 aa, synthetic construct; identical to SEQ ID NO: 235

SEQ ID NO: 302 - protein, 107 aa, synthetic construct [sequence omitted]

SEQ ID NO: 303 - protein, 120 aa, synthetic construct
EVQLQQPGAELVKPGASVKLSCKASGYAFTHYYIYWIKQRPGQGLEWIGGVNPSNGGTHFNEKFKSKATLTVDKSSSTAYMQLSSLTSEDSAVYYCTRSEYDYGLGFAYWGQGTLLTVSS

SEQ ID NO: 304 - protein, 112 aa, synthetic construct
EIVMSQSPSSLAVSAGEKVTMSCKSSQSLLNSRTPKNYLAWYQQKPGQSPKLLIYWASTRKSGVPDRFTGSGSGTDFTLTISSVQAEDLAVYYCKQSYNLLTFGAGTKLELK

SEQ ID NO: 305 - protein, 5 aa, synthetic construct
HYYIY

SEQ ID NO: 306 - protein, 17 aa, synthetic construct
GVNPSNGGTHFNEKFKS

SEQ ID NO: 307 - protein, 11 aa, synthetic construct
SEYDYGLGFAY

SEQ ID NO: 308 - protein, 17 aa, synthetic construct
KSSQSLLNSRTPKNYLA

SEQ ID NO: 309 - protein, 7 aa, synthetic construct
WASTRKS

SEQ ID NO: 310 - protein, 8 aa, synthetic construct
KQSYNLLT

SEQ ID NO: 311 - protein, 30 aa, synthetic construct
EVQLQQPGAELVKPGASVKLSCKASGYAFT

SEQ ID NO: 312 - protein, 14 aa, synthetic construct
WIKQRPGQGLEWIG

SEQ ID NO: 313 - protein, 32 aa, synthetic construct
KATLTVDKSSSTAYMQLSSLTSEDSAVYYCTR

SEQ ID NO: 314 - protein, 11 aa, synthetic construct
WGQGTLLTVSS

SEQ ID NO: 315 - protein, 23 aa, synthetic construct
EIVMSQSPSSLAVSAGEKVTMSC

SEQ ID NO: 316 - protein, 15 aa, synthetic construct
WYQQKPGQSPKLLIY

SEQ ID NO: 317 - protein, 32 aa, synthetic construct
GVPDRFTGSGSGTDFTLTISSVQAEDLAVYYC

SEQ ID NO: 318 - protein, 9 aa, synthetic construct
GAGTKLELK

SEQ ID NO: 319 - protein, 330 aa, synthetic construct
  VARIANT 205: note = K or A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCXVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 320 - protein, 107 aa, synthetic construct [sequence omitted]

SEQ ID NO: 321 - protein, 137 aa, Homo sapiens
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 322 - protein, 5 aa, synthetic construct
GGGGS

SEQ ID NO: 323 - protein, 5 aa, synthetic construct
GSGGS

SEQ ID NO: 324 - protein, 4 aa, synthetic construct
GGGS

SEQ ID NO: 325 - protein, 6 aa, synthetic construct
GSGSGS

SEQ ID NO: 326 - protein, 22 aa, synthetic construct
GSGSGSGSGSGSGSGSGSGSGS

SEQ ID NO: 327 - protein, 15 aa, synthetic construct
GGGGSGGGGSGGGGS

SEQ ID NO: 328 - protein, 12 aa, synthetic construct
GTPTPTPTPTGE

SEQ ID NO: 329 - protein, 17 aa, synthetic construct
AEAAAKEAAAKEAAAKA

SEQ ID NO: 330 - protein, 5 aa, synthetic construct
PAPAP

(19) United States
(12) Patent Application Publication
(10) Pub. No.: US 2026/0263645 A1
(43) Pub. Date: Sep. 10, 2026

(54) MULTIFUNCTIONAL ANTIBODIES

(71) Applicant: Telix Pharmaceuticals (Innovations) Pty Ltd, Victoria (AU)

(72) Inventors: Kwame TWUMASI-BOATENG, Victoria (AU); Michael Paul WHEATCROFT, Victoria (AU); David CHIN, Victoria (AU)

(21) Appl. No.: 19/163,361

(22) PCT Filed: Mar. 8, 2024
(86) PCT No.: PCT/AU2024/050198

§ 371 Date: Sep. 8, 2025

(30) Foreign Application Priority Data
Mar. 9, 2023 (AU) 2023900642

(51) Int. Cl. A61K 51/10; A61K 47/68; A61P 35/00; C07K 14/71; C07K 16/28; C07K 16/30; C07K 16/40

(52) U.S. Cl. CPC A61K 51/1087; A61K 47/6851; A61K 47/6869; A61P 35/00; C07K 14/71; C07K 16/2863; C07K 16/3069; C07K 16/40; C07K 2317/622

(57) ABSTRACT

The invention relates to molecules comprising a tumour antigen binding protein and an extracellular domain (ECD) or ligand binding fragment of a transforming growth factor β receptor (TGFβR), and compositions and uses thereof.

### FIELD OF THE INVENTION

The invention relates to molecules, compositions and methods for treating cancer.

### RELATED APPLICATION

This application claims priority from Australian provisional application AU 2023900642, the entire contents of which are hereby incorporated by reference.

### BACKGROUND OF THE INVENTION

Radiation and other treatments for cancer can destroy cells and trigger inflammation that initiates immune responses. Compensatory mechanisms exist in the body to promote wound-healing and limit inflammation following an insult like radiation. While these mechanisms can be important in normal tissue, in cancer tissue they can prematurely shut-off the anti-tumour immune response and can also make tumours resistant to subsequent rounds of radiation.

There remains a need for new or improved molecules and methods for treating cancer.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention relates to molecules comprising a first moiety in the form of a tumour antigen binding protein and second moiety in the form of an extracellular domain (ECD) or ligand binding fragment of a transforming growth factor β receptor (TGFβR).

As described herein, the tumour antigen binding protein may be in the form of:
(i) a single chain Fv fragment (scFv);
(ii) a dimeric scFv (di-scFv);
(iii) one of (i) or (ii) linked to a constant region of an antibody, Fc or a heavy chain constant domain (CH) 2 and/or CH3.

Further, as described herein, the antigen binding protein may be in the form of:
(i) a diabody;
(ii) a triabody;
(iii) a tetrabody;
(iv) a Fab;
(v) a F(ab′)2;
(vi) a Fv;
(vii) a bispecific antibody or other form of multispecific antibody;
(viii) one of (i) to (vii) linked to a constant region of an antibody, Fc or a heavy chain constant domain (CH) 2 and/or CH3.

Accordingly, in any embodiment, the molecule may comprise a tumour antigen binding protein in the form of: an antigen binding domain of an immunoglobulin, an antibody, a bispecific or multispecific antibody, an antibody fragment, a single chain variable fragment (scFv), a bivalent or multivalent scFv, or a Fc-containing polypeptide.

In any embodiment, the tumour antigen binding protein is an antibody or antigen binding fragment thereof, and the ECD or ligand binding fragment of a TGFβR is joined to the C terminus of the heavy chain of the antibody or antigen binding fragment thereof.

Alternatively, the tumour antigen binding protein may be an antibody or antigen binding fragment thereof, wherein the ECD or ligand binding fragment of a TGFβR is joined to the C terminus of the light chain of the antibody or antigen binding fragment thereof.

Alternatively, the tumour antigen binding protein may be an antibody or antigen binding fragment thereof, wherein the ECD or ligand binding fragment of a TGFβR is joined to the C terminus of the heavy chain of the antibody or antigen binding fragment thereof.

In alternative embodiments, the ECD or ligand binding fragment of a TGFβR may be joined to any non-antigen binding region of the tumour antigen binding protein. For example in instances wherein the tumour antigen binding protein is an antibody or Fab, the ECD or ligand binding fragment of a TGFβR may be joined to any amino acid of any constant region of the protein or any region of a variable domain which is not directly involved in antigen binding.

In any embodiment, the ECD or ligand binding fragment of a TGFβR may be joined directly to the tumour antigen binding protein, or may be joined via a peptide linker, a carbohydrate (eg polyethylene glycol based linker or similar) or chemical conjugation.

It will be appreciated that the tumour antigen binding protein of the molecule may bind to any antigen which is associated with or specific for tumour cells, as further described herein. Non-limiting examples of antigens which are associated with (TAAs) or which are specific for tumour cells (TSAs) may include: 17-IA-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CD1, CD1a, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD79b, CD80, CD83, CD95, CD123, CD126, CD132, CD133, CD138, CD147, CD154, CD171, CDC27, CDK-4/m, CDKN2A, CEA, CEACAM5, CEACAM6, complement factors (such as C3, C3a, C3b, C5a and C5), colon-specific antigen-p (CSAp), c-Met, CTLA-4, CXCR4, CXCR7, DAM, Dickkopf-related protein (DKK), ED-B fibronectin, EGFR, EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gplOO, GPC3, GRO-13, HLA-DR, HM1.24, human chorionic gonadotropin (HCG) and its subunits, HER2, HER3, HMGB-1, hypoxia inducible factor (HIF-1), HIF-la, HSP70-2M, HST-2, la, IFN-gamma, IFN-alpha, IFN-beta, IFN-X, IL-4R, IL-6R, IL-13R, IL13Ralpha2, IL-15R, IL-17R, IL-18R, ILGF, ILGF-1R, insulin-like growth factor-1 (IGF-1), IGF-1R, integrin avP3, integrin α5β1, KC4-antigen, killer-cell immunoglobulin-like receptor (KIR), Kras, KS-1-antigen, KS1-4, La/SSB, LDR/FUT, Le1, macrophage migration inhibitory factor (MIF), MAGE, MAGE-3, MART-1, MART-2, mCRP, MCP-1, melanoma glycoprotein, mesothelin, MIP-1 A, MIP-1B, MIF, mucins (such as MUC1, MUC2, MUC3, MUC4, MUC5ac, MUC13, MUC16, MUM-1/2 and MUM-3), NCA66, NCA95, NCA90, NY-ESO-1, PAM4 antigen, pancreatic cancer mucin, PD-1, PD-L1, PD-1 receptor, placental growth factor, platelet-derived growth factor receptor alpha (PDGFRa), p53, PLAGL2, prostatic acid phosphatase, PSA, PRAME, PSMA, P1GF, RS5, RANTES, SAGE, 5100, survivin, survivin-2B, T101, TAC, TAG-72, tenascin, Thomson-Friedenreich antigens, Tn antigen, TNF-alpha, tumour necrosis antigens, TRAG-3, TRAIL receptors, VEGF, VEGFR and WT-1. It will therefore be appreciated that in any embodiment, the tumour antigen binding protein may be one that is capable of specifically binding to any such antigen.

Optionally, the tumour antigen binding protein may be one selected from, or may comprise an antigen binding domain derived from any one of the following: APOMAB (DAB4), atezolizumab, avelumab, bevacizumab, cemiplimab, cetuximab, dataumumab, dinutuximab, durvalumab, elotuzumab, girentuximab, ipilimumab, isatuximab, J591 or huJ591, mogamulizumab, nectimumumab, nivolumab, obinutuzumab, ofatumumab, olaratumab, panitumumab, pembrolizumab, pertizimab, ramucirumab, rituximab, trastuzumab.

Preferably, the tumour antigen binding protein is capable of binding a tumour antigen expressed by a solid cancer.

In certain embodiments, the tumour antigen is not EGFR or EGFRvIII.

In certain embodiment, the tumour antigen is no PD-1 or PD-L1.

In preferred embodiments of the invention, the tumour antigen binding protein may be selected from one that binds to prostate specific membrane antigen (PSMA), carbonic anhydrase IX (CAIX), PDGFRa or La/SSB. Exemplary amino acid sequences of tumour antigen binding proteins for binding to PSMA, CAIX, PDGFRα and La/SSB are further described herein, including in Tables 1, 2 and 5 and 6 herein, respectively.

In any embodiment, the second moiety of the molecule is in the form of an extracellular domain (ECD) or ligand binding fragment of a transforming growth factor β receptor (TGFβR), wherein the TGFβR is a type I (TGFβRI), type II (TGFβRII) or type III (TGFβRIII) TGFβR. In preferred embodiments, the second moiety of the molecule comprises an amino acid sequence that is derived from or comprises the ECD, or ligand binding domain of a type II TGF βR (TGFβRII).

Exemplary amino acid sequences of ECDs and fragments thereof, of TGFβR and isoforms thereof, are further described herein, including in Table 3.

In any embodiment, the tumour antigen binding domain and the ECD or ligand binding fragment of a TGFβR are joined via a linker.

In any embodiment, any molecule of the invention may be conjugated to a therapeutic agent. The therapeutic agent may be conjugated to the molecule directly or indirectly, e.g. by halogenation of amino acid residues. Preferably, the therapeutic agent is indirectly conjugated to the molecule by way of a linker or chelator moiety. In one example, the molecule is conjugated to a chelating moiety, selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq and HOPO or other chelating agent as described herein. In preferred embodiments, the therapeutic agent is a radionuclide.

In any embodiment, the radionuclide may be an alpha emitting radionuclide or a betta emitting radionuclide. As used herein, the terms radioactive isotope, radioisotope, radionuclide, and radioactive nuclide may be used interchangeably.

The radionuclide may be selected from the group consisting of: actinium-225 (225Ac), astatine-211 (211At), bismuth-212 and bismuth-213 (²¹²Bi, ²¹³Bi), copper-67 (⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²⁴Ra), samarium-153 (¹⁵³Sm), scandium-47 (⁴⁷Sc), strontium-90 (⁹⁰Sr), and yttrium-90 (⁹⁰Y). In some embodiments, the radionuclide conjugated with the molecule is lutetium-¹⁷⁷.

In any embodiment, the therapeutic agent, preferably a radioisotope, may be conjugated to the tumour antigen binding protein moiety of the molecule. In alternative or additional embodiments, the therapeutic agent, preferably a radioisotope, may be conjugated to the moiety of the molecule that comprises the ECD or ligand binding fragment of a TGFβR. In alternative embodiments, the therapeutic agent, preferably a radioisotope, may be non-site specifically conjugated to the molecule.

The invention also provides a bioconjugate molecule comprising a first moiety in the form of a tumour antigen binding protein and second moiety in the form of an extracellular domain (ECD) or ligand binding fragment of a transforming growth factor β receptor (TGFβR), suitable for radiolabelling with a therapeutic agent, preferably a therapeutic radionuclide.

In one aspect, the present invention provides a bioconjugate comprising a molecule of the invention conjugated to any chelating moiety or linker group suitable for further conjugation to a radionuclide. In particular, the chelating agent or linker group is capable of indirectly conjugating a radionuclide to the molecule.

Optionally, the chelating agent is selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N′″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8), 11-tetraacetic acid, Te2A (4,11-bis(carboxymethyl)-1,4,8,11-tetraazabicyclo[6.6.2]hexadecane), HBED, DFO (Desferrioxamine), DFOsq (DFO-squaramide) and HOPO (3,4,3-(LI-1,2-HOPO) or any other chelating agent as described herein.

In any embodiment, the bioconjugate comprises a molecule of the invention conjugated to a chelating moiety, selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq and HOPO.

In any embodiment, the bioconjugate comprises a molecule of the invention conjugated to a bifunctional linker, for example, bromoacetyl, thiols, succinimide ester, TFP ester, a maleimide, or using any amine or thiol-modifying chemistry known in the art.

In any embodiment, the chelating agent or linker group may be conjugated to the tumour antigen binding protein moiety of the molecule. In alternative or additional embodiments, the chelating agent or linker group may be conjugated to the moiety of the molecule that comprises the ECD or ligand binding fragment of a TGFβR.

The invention also provides a nucleic acid encoding a molecule or bioconjugate of the invention, or components thereof, such as the tumour antigen binding protein, immunoglobulin variable domain, antibody, dab, di-scFv, scFv, Fab, Fab′, F(ab′)2, Fv fragment, diabody, triabody, tetrabody, linear antibody, single-chain antibody molecule, or multispecific antibody, fusion protein or conjugate as described herein.

In one example, such a nucleic acid is included in an expression construct in which the nucleic acid is operably linked to a promoter. Such an expression construct can be in a vector, e.g., a plasmid.

In examples of the invention directed to single polypeptide chain antigen binding protein, the expression construct may comprise a promoter linked to a nucleic acid encoding that polypeptide chain.

In examples directed to multiple polypeptide chains that form an antigen binding protein, an expression construct comprises a nucleic acid encoding a polypeptide comprising, e.g., a VH operably linked to a promoter and a nucleic acid encoding a polypeptide comprising, e.g., a VL operably linked to a promoter.

In another example, the expression construct is a bicistronic expression construct, e.g., comprising the following operably linked components in 5′ to 3′ order:
(i) a promoter
(ii) a nucleic acid encoding a first polypeptide;
(iii) an internal ribosome entry site; and
(iv) a nucleic acid encoding a second polypeptide, wherein the first polypeptide comprises a VH (and optionally an extracellular domain (ECD) or ligand binding fragment of a TGFβR) and the second polypeptide comprises a VL (and optionally an extracellular domain (ECD) or ligand binding fragment of a TGFβR), or vice versa.

The present invention also contemplates separate expression constructs one of which encodes a first polypeptide comprising a VH and another of which encodes a second polypeptide comprising a VL. For example, the present invention also provides a composition comprising:
(i) a first expression construct comprising a nucleic acid encoding a polypeptide comprising a VH operably linked to a promoter; optionally further encoding a polypeptide comprising extracellular domain (ECD) or ligand binding fragment of a TGFβR; and
(ii) a second expression construct comprising a nucleic acid encoding a polypeptide comprising a VL operably linked to a promoter (optionally further encoding a polypeptide comprising extracellular domain (ECD) or ligand binding fragment of a TGFβR).

The invention provides a cell comprising a vector or nucleic acid described herein. Preferably, the cell is isolated, substantially purified or recombinant. In one example, the cell comprises the expression construct of the invention or:
(i) a first expression construct comprising a nucleic acid encoding a polypeptide comprising a VH operably linked to a promoter (optionally further encoding a polypeptide comprising extracellular domain (ECD) or ligand binding fragment of a TGFβR); and
(ii) a second expression construct comprising a nucleic acid encoding a polypeptide comprising a VL operably linked to a promoter (optionally further encoding a polypeptide comprising extracellular domain (ECD) or ligand binding fragment of a TGFβR);
   wherein the first and second polypeptides associate to form a molecule of the present invention.

Examples of cells of the present invention include bacterial cells, yeast cells, insect cells or mammalian cells.

The present invention also provides compositions comprising the aforementioned molecules of the invention. Optionally, the compositions may comprise one or more pharmaceutically acceptable carriers or excipients.

In one aspect, the present invention provides a composition comprising a molecule of the invention and a treatment for cancer that is suspected or known to cause an increase in TGFβ activity in the tumour microenvironment when administered to a subject.

Preferably, the treatment for cancer is an antibody or antibody fragment thereof that binds to or specifically binds to an antigen expressed by a cancer, optionally conjugated with a radionuclide. In such embodiments, the antibody or antibody fragment thereof may be for binding to the same cancer antigen as the molecule of the invention.

For example, in one embodiment, the composition comprises i) a molecule comprising first moiety in the form of a tumour antigen binding protein for binding to CAIX and second moiety in the form of an extracellular domain (ECD) of a transforming growth factor β receptor (TGFβR) and ii) an antibody or antigen binding fragment thereof for binding to CAIX. Optionally the molecule of i) comprises a radionuclide but the antibody of ii) does not. Alternatively, the molecule of i) may not comprise a radionuclide and the antibody of ii) comprises a radionuclide. Alternatively, the molecule of i) and the antibody of ii) each comprise a radionuclide.

In another example, in one embodiment, the composition comprises i) a molecule comprising first moiety in the form of a tumour antigen binding protein for binding to PSMA and second moiety in the form of an extracellular domain (ECD) of a transforming growth factor β receptor (TGFβR) and ii) an antibody or antigen binding fragment thereof for binding to PSMA. Optionally the molecule of i) comprises a radionuclide but the antibody of ii) does not. Alternatively, the molecule of i) may not comprise a radionuclide and the antibody of ii) comprises a radionuclide. Alternatively, the molecule of i) and the antibody of ii) each comprise a radionuclide.

In another example, in one embodiment, the composition comprises i) a molecule comprising first moiety in the form of a tumour antigen binding protein for binding to PDGFRα and second moiety in the form of an extracellular domain (ECD) of a transforming growth factor β receptor (TGFβR) and ii) an antibody or antigen binding fragment thereof for binding to PDGFRα. Optionally the molecule of i) comprises a radionuclide but the antibody of ii) does not. Alternatively, the molecule of i) may not comprise a radionuclide and the antibody of ii) comprises a radionuclide. Alternatively, the molecule of i) and the antibody of ii) each comprise a radionuclide.

In another example, in one embodiment, the composition comprises i) a molecule comprising first moiety in the form of a tumour antigen binding protein for binding to La/SSB and second moiety in the form of an extracellular domain (ECD) of a transforming growth factor β receptor (TGFβR) and ii) an antibody or antigen binding fragment thereof for binding to LA/SSB. Optionally the molecule of i) comprises a radionuclide but the antibody of ii) does not. Alternatively, the molecule of i) may not comprise a radionuclide and the antibody of ii) comprises a radionuclide. Alternatively, the molecule of i) and the antibody of ii) each comprise a radionuclide.

The present invention also provides various methods of use and uses of the molecules and compositions described herein.

In one aspect, the present invention provides a method of treating, preventing or minimising progression of cancer in a subject comprising administering to the subject a molecule comprising an antigen binding protein that binds to or specifically binds to an antigen expressed by the cancer, wherein the molecule further comprises an ECD or ligand binding fragment of a TGFβR, thereby treating, preventing or minimising progression of cancer in the subject. Optionally, the molecule may be conjugated to a radionuclide.

The present invention also provides various uses of the molecules and compositions described herein. The present invention also provides a method of treating, preventing or minimising progression of cancer in a subject comprising administering to the subject a composition or molecule of the invention, wherein the composition or molecule comprises an antigen binding protein that binds to or specifically binds to an antigen expressed by the cancer, wherein the molecule further comprises an ECD or ligand binding fragment of a TGFβR, thereby treating, preventing or minimising progression of cancer in the subject. Optionally, the molecule may be conjugated to a radionuclide.

The present invention further provides a method of inhibiting TGFβ activity in a subject with cancer, the method comprising administering to the subject, a molecule or composition of the invention, thereby inhibiting TGFβ activity in the cancer. Optionally, the cancer may be one that has high levels of baseline TGFβ expression or activity in the tumour microenvironment. Optionally, the subject may have received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the method may comprise administering a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

The present invention further provides a method inhibiting or preventing cancer treatment-related fibrosis in a subject, the method comprising administering a molecule or composition of the invention, thereby treating the cancer in the subject, optionally wherein the subject has received a prior treatment for the cancer which increases expression of TGFβ in the subject. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

In a particularly preferred embodiment, the present invention provides a method for reducing or inhibiting radiation-induced TGFβ activity in a subject receiving or requiring radiation treatment for cancer, wherein the method comprises administering a molecule or composition of the invention to the subject, thereby reducing or inhibiting radiation-induced TGFβ activity in the subject. The radiation treatment may be provided in the form of external beam radiation or molecular targeted radiation. In any embodiment, the source of the molecular targeted radiation is a molecule or composition of the invention.

In further embodiments, the present invention provides a method for enhancing or increasing the likelihood of success of treatment with an immune checkpoint inhibitor in a subject, wherein the method comprises administering a molecule or composition of the invention to the subject, thereby enhancing or increasing the likelihood of success of treatment with an immune checkpoint inhibitor in a subject. Optionally, the cancer may be one that has high levels of baseline TGFβ expression or activity in the tumour microenvironment. Optionally, the subject has received a prior treatment for cancer, wherein the prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

The present invention also provides a method of treating, preventing or minimising progression of cancer characterised by the expression of carbonic anhydrase IX (CAIX) in a subject, comprising administering to the subject a molecule comprising an antigen binding protein that binds to or specifically binds to CAIX, wherein the molecule further comprises an ECD or ligand binding fragment of a TGFβR, thereby treating, preventing or minimising progression of the cancer in the subject. Optionally, the molecule may be conjugated to a radionuclide.

The present invention also provides a method of treating, preventing or minimising progression of cancer characterised by the expression of prostate specific membrane antigen (PSMA) in a subject, comprising administering to the subject a molecule comprising an antigen binding protein that binds to or specifically binds to PSMA, wherein the molecule further comprises an ECD or ligand binding fragment of a TGFβR, thereby treating, preventing or minimising progression of the cancer in the subject. Optionally, the molecule may be conjugated to a radionuclide.

The present invention also provides a method of treating, preventing or minimising progression of cancer characterised by the expression of platelet derived growth factor receptor alpha (PDGFRα) in a subject, comprising administering to the subject a molecule comprising an antigen binding protein that binds to or specifically binds to PDGFRα, wherein the molecule further comprises an ECD or ligand binding fragment of a TGFβR, thereby treating, preventing or minimising progression of the cancer in the subject. Optionally, the molecule may be conjugated to a radionuclide.

The present invention also provides a method of treating, preventing or minimising progression of cancer characterised by the expression of La/SSB protein in a subject, comprising administering to the subject a molecule comprising an antigen binding protein that binds to or specifically binds to La/SSB, wherein the molecule further comprises an ECD or ligand binding fragment of a TGFβR, thereby treating, preventing or minimising progression of the cancer in the subject. Optionally, the molecule may be conjugated to a radionuclide.

The present invention further provides a method of inhibiting TGFβ activity in a subject with cancer characterised by the expression of carbonic anhydrase IX (CAIX), the method comprising administering to the subject a molecule comprising an antigen binding protein that binds to or specifically binds to carbonic anhydrase IX (CAIX), wherein the molecule further comprises an ECD or ligand binding fragment of a TGFβR, thereby inhibiting TGFβ activity in the cancer. Optionally, the cancer may be one that has high levels of baseline TGFβ expression or activity in the tumour microenvironment. Optionally, the subject may have received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be administered a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

The present invention further provides a method inhibiting or preventing cancer treatment-related fibrosis in a subject, the method comprising administering a molecule comprising an antigen binding protein that binds to or specifically binds to carbonic anhydrase IX (CAIX), wherein the molecule further comprises an ECD or ligand binding fragment of a TGFβR, thereby treating the cancer in the subject, optionally wherein the subject has received a prior treatment for the cancer which increases expression of TGFβ in the subject. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

In a particularly preferred embodiment, the present invention provides a method for reducing or inhibiting radiation-induced TGFβ activity in a subject receiving or requiring radiation treatment for cancer, wherein the method comprises administering a molecule comprising an antigen binding protein that binds to or specifically binds to carbonic anhydrase IX (CAIX), wherein the molecule further comprises an ECD or ligand binding fragment of a TGFβR, thereby reducing or inhibiting radiation-induced TGFβ activity in the subject. The radiation treatment may be provided in the form of external beam radiation or molecular targeted radiation. In any embodiment, the source of the molecular targeted radiation is a molecule or composition of the invention.

In further embodiments, the present invention provides a method for enhancing or increasing the likelihood of success of treatment with an immune checkpoint inhibitor in a subject, wherein the method comprises administering a molecule comprising an antigen binding protein that binds to or specifically binds to carbonic anhydrase IX (CAIX), wherein the molecule further comprises an ECD or ligand binding fragment of a TGFβR, to the subject, thereby enhancing or increasing the likelihood of success of treatment with an immune checkpoint inhibitor in a subject. Optionally, the cancer may be one that has high levels of baseline TGFβ expression or activity in the tumour microenvironment. Optionally, the subject has received a prior treatment for cancer, wherein the prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

The present invention further provides a method of inhibiting TGFβ activity in a subject with cancer characterised by the expression of PSMA, the method comprising administering to the subject a molecule comprising an antigen binding protein that binds to or specifically binds to PSMA, wherein the molecule further comprises an ECD or ligand binding fragment of a TGFβR, thereby inhibiting TGFβ activity in the cancer. Optionally, the cancer may be one that has high levels of baseline TGFβ expression or activity in the tumour microenvironment. Optionally, the subject may have received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be administered a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

The present invention further provides a method inhibiting or preventing cancer treatment-related fibrosis in a subject, the method comprising administering a molecule comprising an antigen binding protein that binds to or specifically binds to prostate specific membrane antigen (PSMA), wherein the molecule further comprises an ECD or ligand binding fragment of a TGFβR, thereby treating the cancer in the subject, optionally wherein the subject has received a prior treatment for the cancer which increases expression of TGFβ in the subject. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

In a particularly preferred embodiment, the present invention provides a method for reducing or inhibiting radiation-induced TGFβ activity in a subject receiving or requiring radiation treatment for cancer, wherein the method comprises administering a molecule comprising an antigen binding protein that binds to or specifically binds to prostate specific membrane antigen (PSMA), wherein the molecule further comprises an ECD or ligand binding fragment of a TGFβR, thereby reducing or inhibiting radiation-induced TGFβ activity in the subject. The radiation treatment may be provided in the form of external beam radiation or molecular targeted radiation. In any embodiment, the source of the molecular targeted radiation is a molecule or composition of the invention.

In further embodiments, the present invention provides method for enhancing or increasing the likelihood of success of treatment with an immune checkpoint inhibitor in a subject, wherein the method comprises administering a molecule comprising an antigen binding protein that binds to or specifically binds to prostate specific membrane antigen (PSMA), wherein the molecule further comprises an ECD or ligand binding fragment of a TGFβR, to the subject, thereby enhancing or increasing the likelihood of success of treatment with an immune checkpoint inhibitor in a subject. Optionally, the cancer may be one that has high levels of baseline TGFβ expression or activity in the tumour microenvironment. Optionally, the subject has received a prior treatment for cancer, wherein the prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

The present invention further provides a method of inhibiting TGFβ activity in a subject with cancer characterised by the expression of PDGFRα, the method comprising administering to the subject a molecule comprising an antigen binding protein that binds to or specifically binds to PDGFRα, wherein the molecule further comprises an ECD or ligand binding fragment of a TGFβR, thereby inhibiting TGFβ activity in the cancer. Optionally, the cancer may be one that has high levels of baseline TGFβ expression or activity in the tumour microenvironment. Optionally, the subject may have received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be administered a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

The present invention further provides a method inhibiting or preventing cancer treatment-related fibrosis in a subject, the method comprising administering a molecule comprising an antigen binding protein that binds to or specifically binds to PDGFRα, wherein the molecule further comprises an ECD or ligand binding fragment of a TGFβR, thereby treating the cancer in the subject, optionally wherein the subject has received a prior treatment for the cancer which increases expression of TGFβ in the subject. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

In a particularly preferred embodiment, the present invention provides a method for reducing or inhibiting radiation-induced TGFβ activity in a subject receiving or requiring radiation treatment for cancer, wherein the method comprises administering a molecule comprising an antigen binding protein that binds to or specifically binds to PDGFRα, wherein the molecule further comprises an ECD or ligand binding fragment of a TGFβR, thereby reducing or inhibiting radiation-induced TGFβ activity in the subject. The radiation treatment may be provided in the form of external beam radiation or molecular targeted radiation. In any embodiment, the source of the molecular targeted radiation is a molecule or composition of the invention.

In further embodiments, the present invention provides method for enhancing or increasing the likelihood of success of treatment with an immune checkpoint inhibitor in a subject, wherein the method comprises administering a molecule comprising an antigen binding protein that binds to or specifically binds to PDGFRα wherein the molecule further comprises an ECD or ligand binding fragment of a TGFβR, to the subject, thereby enhancing or increasing the likelihood of success of treatment with an immune checkpoint inhibitor in a subject. Optionally, the cancer may be one that has high levels of baseline TGFβ expression or activity in the tumour microenvironment. Optionally, the subject has received a prior treatment for cancer, wherein the prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

The present invention further provides a method of inhibiting TGFβ activity in a subject with cancer characterised by the expression of La/SSB, the method comprising administering to the subject a molecule comprising an antigen binding protein that binds to or specifically binds to La/SSB, wherein the molecule further comprises an ECD or ligand binding fragment of a TGFβR, thereby inhibiting TGFβ activity in the cancer. Optionally, the cancer may be one that has high levels of baseline TGFβ expression or activity in the tumour microenvironment. Optionally, the subject may have received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the subject may be administered a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

The present invention further provides a method inhibiting or preventing cancer treatment-related fibrosis in a subject, the method comprising administering a molecule comprising an antigen binding protein that binds to or specifically binds to La/SSB, wherein the molecule further comprises an ECD or ligand binding fragment of a TGFβR, thereby treating the cancer in the subject, optionally wherein the subject has received a prior treatment for the cancer which increases expression of TGFβ in the subject. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

In any embodiment, the compositions or molecules of the invention may be for use as neoadjuvant agent(s) for administration to a patient prior to treatment with a therapy as described herein.

In any embodiment, the composition or molecules of the invention may be for use as adjuvant agent(s) for administration to a patient after treatment with a therapy as described herein.

In a particularly preferred embodiment, the present invention provides a method for reducing or inhibiting radiation-induced TGFβ activity in a subject receiving or requiring radiation treatment for cancer, wherein the method comprises administering a molecule comprising an antigen binding protein that binds to or specifically binds to La/SSB, wherein the molecule further comprises an ECD or ligand binding fragment of a TGFβR, thereby reducing or inhibiting radiation-induced TGFβ activity in the subject. The radiation treatment may be provided in the form of external beam radiation or molecular targeted radiation. In any embodiment, the source of the molecular targeted radiation is a molecule or composition of the invention.

In further embodiments, the present invention provides method for enhancing or increasing the likelihood of success of treatment with an immune checkpoint inhibitor in a subject, wherein the method comprises administering a molecule comprising an antigen binding protein that binds to or specifically binds to La/SSB, wherein the molecule further comprises an ECD or ligand binding fragment of a TGFβR, to the subject, thereby enhancing or increasing the likelihood of success of treatment with an immune checkpoint inhibitor in a subject. Optionally, the cancer may be one that has high levels of baseline TGFβ expression or activity in the tumour microenvironment. Optionally, the subject has received a prior treatment for cancer, wherein the prior treatment for cancer may be selected from the group consisting of: treatment with external beam radiation (EBR), treatment with a chemotherapeutic agent, surgery or resection of the tumour, treatment with an immunomodulatory agent, including a CPI, treatment with molecular targeted radiation (MTR), treatment with a cell therapy, such as CAR T therapy.

In any embodiment, an antigen binding protein for binding to any of CAIX, PSMA, PDGFRα or LA/SSB may be one as further described herein. In any aspect or embodiment described herein, the antigen binding protein that binds to or specifically binds to carbonic anhydrase IX (CAIX) may be an antibody against CAIX. In some embodiments, the anti-CAIX antibody is girentuximab or a derivative thereof, including humanised forms of girentuximab. In some embodiments, the antigen binding protein that binds to or specifically binds to PSMA may be derived from J591, or huJ591, as is further described herein. In some embodiments, the antigen binding protein that binds to PDGFRα is olaratumab, or a derivative thereof. In some embodiments, the antigen binding protein that binds to or specifically binds to LA/SSB may be APOMAB or a derivative thereof, including humanised forms of APOMAB.

In any embodiment, the method further comprises administering a treatment for cancer that is suspected or known to cause an increase in TGFβ activity in the tumour microenvironment when administered to a subject. In preferred embodiments, the treatment for cancer is a molecular targeted radionuclide (MTR), such as in the form of an antibody or antibody fragment thereof that binds to or specifically binds to an antigen expressed by a cancer conjugated with a radionuclide. Optionally, the molecule of the invention does not comprise a radionuclide.

Accordingly, in further embodiments the present invention may comprise:
administering to the subject, a molecule or composition of the invention, optionally wherein the molecule does not comprise a radionuclide; and
administering to the subject a treatment for cancer that is suspected or known to cause an increase in TGFβ activity in the tumour microenvironment, preferably wherein the treatment for cancer is molecular targeted radiation (MTR), such as in the form of an antibody or antibody fragment thereof that binds to or specifically binds to an antigen expressed by a cancer, optionally conjugated with a radionuclide.

In some embodiments, the antibody or antibody fragment thereof may be for binding to the same cancer antigen as the molecule of the invention.

For example, in one embodiment, the method comprises administering i) a molecule comprising first moiety in the form of a tumour antigen binding protein for binding to CAIX and second moiety in the form of an extracellular domain (ECD) of a transforming growth factor β receptor (TGFβR) and ii) an antibody or antigen binding fragment thereof for binding to CAIX. Optionally the molecule of i) comprises a radionuclide but the antibody of ii) does not. Alternatively, the molecule of i) may not comprise a radionuclide and the antibody of ii) comprises a radionuclide.

In another example, in one embodiment, the method comprises administering i) a molecule comprising first moiety in the form of a tumour antigen binding protein for binding to PSMA and second moiety in the form of an extracellular domain (ECD) of a transforming growth factor β receptor (TGFβR) and ii) an antibody or antigen binding fragment thereof for binding to PSMA. Optionally the molecule of i) comprises a radionuclide but the antibody of ii) does not. Alternatively, the molecule of i) may not comprise a radionuclide and the antibody of ii) comprises a radionuclide. Alternatively the molecule of i) and the antibody of ii) each comprise a radionuclide.

In another example, in one embodiment, the method comprises administering i) a molecule comprising first moiety in the form of a tumour antigen binding protein for binding to PDGFRα and second moiety in the form of an extracellular domain (ECD) of a transforming growth factor β receptor (TGFβR) and ii) an antibody or antigen binding fragment thereof for binding to PDGFRα. Optionally the molecule of i) comprises a radionuclide but the antibody of ii) does not. Alternatively, the molecule of i) may not comprise a radionuclide and the antibody of ii) comprises a radionuclide. Alternatively the molecule of i) and the antibody of ii) each comprise a radionuclide.

In another example, in one embodiment, the method comprises administering i) a molecule comprising first moiety in the form of a tumour antigen binding protein for binding to La/SSB and second moiety in the form of an extracellular domain (ECD) of a transforming growth factor β receptor (TGFβR) and ii) an antibody or antigen binding fragment thereof for binding to LA/SSB. Optionally the molecule of i) comprises a radionuclide but the antibody of ii) does not. Alternatively, the molecule of i) may not comprise a radionuclide and the antibody of ii) comprises a radionuclide. Alternatively the molecule of i) and the antibody of ii) each comprise a radionuclide.

In any aspect or embodiment described herein, an immune checkpoint inhibitor (CPI) may be a PD-1, PD-L1, CTLA-4, TIGIT, VISTA, LAG-3, TIM-3 or CD47 checkpoint inhibitor. In any embodiment, the checkpoint inhibitor may be an antibody or antigen binding fragment thereof, a protein, a peptide or a small molecule. In some embodiments, the checkpoint inhibitor is an inhibitor of PD-1, PD-L1, CTLA-4 TIGIT, VISTA, LAG-3, TIM-3 or CD47 in the form of an antibody or antigen binding fragment thereof. In some embodiments, the checkpoint inhibitor is an inhibitor of PD-1, PD-L1, CTLA-4 TIGIT, VISTA, LAG-3, TIM-3 or CD47 in the form of a peptide. In some embodiments, the checkpoint inhibitor is an inhibitor of PD-1. In some embodiments, the checkpoint inhibitor is an inhibitor of CTLA-4. In some embodiments, immune checkpoint inhibitor therapy involves administering inhibitors of PD-1 and CTLA-4. In some embodiments, the checkpoint inhibitor is an inhibitor of TIGIT. In some embodiments, the checkpoint inhibitor is an inhibitor of VISTA. In some embodiments, the checkpoint inhibitor is an inhibitor of LAG-3. In some embodiments the checkpoint inhibitor is an inhibitor of TIM-3. In some embodiments, the checkpoint inhibitor is an inhibitor of CD47.

In any embodiment of the invention, the methods described herein further comprise identifying a subject having cancer. In an embodiment, the cancer may be pre-cancerous or non-metastatic. In another embodiment, the cancer may be malignant or metastatic.

In any embodiment of the invention, the immune checkpoint inhibitor (CPI) may be a PD-1, PD-L1 or a CTLA-4 checkpoint inhibitor. In any aspect, the checkpoint inhibitor is an antibody. In some embodiments, the checkpoint inhibitor is an inhibitor of PD-1, PD-L1 or CTLA-4 in the form of an antibody. In some embodiments, the checkpoint inhibitor is an inhibitor of PD-1. In some embodiments, the checkpoint inhibitor is an inhibitor of CTLA-4. In some embodiments, immune checkpoint inhibitor therapy involves administering inhibitors of PD-1 and CTLA-4.

The present invention also provide a composition or molecule as described herein, for use in:
a method of treating, preventing or minimising progression of cancer in a subject;
a method of inhibiting TGFβ activity in a subject with cancer;
a method for reducing or inhibiting radiation-induced TGFβ activity in a subject receiving or requiring radiation treatment for cancer;
a method inhibiting or preventing cancer treatment-related fibrosis in a subject; and/or
a method for enhancing or increasing the likelihood of success of treatment with an immune checkpoint inhibitor in a subject
   wherein the composition or molecule comprises an antigen binding protein that binds to or specifically binds to an antigen expressed by the cancer and wherein the molecule further comprises an ECD or ligand binding fragment of a TGFβR. Optionally, the molecule may be conjugated to a radionuclide.

In any embodiment, the composition or molecule for use as described above, may be for use after the subject has received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

In any embodiment, the composition or molecule for use as described above, may be for use prior to the subject receiving a treatment for the cancer, wherein the treatment is suspected of causing, or causes, an increase in TGFβ activity in the tumour microenvironment. The treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

Optionally, the use may be for the treatment of a subject that may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the use may be for administration of a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

The present invention also provide a use of a molecule or bioconjugate as described herein, for use in the manufacture of a medicament for:
treating, preventing or minimising progression of cancer in a subject;
inhibiting TGFβ activity in a subject with cancer;
reducing or inhibiting radiation-induced TGFβ activity in a subject receiving or requiring radiation treatment for cancer;
inhibiting or preventing cancer treatment-related fibrosis in a subject; and/or
enhancing or increasing the likelihood of success of treatment with an immune checkpoint inhibitor in a subject wherein the molecule or bioconjugate comprises an antigen binding protein that binds to or specifically binds to an antigen expressed by the cancer and wherein the molecule further comprises an ECD or ligand binding fragment of a TGFβR. Optionally, the molecule may be conjugated to a radionuclide.

The present invention also provide a composition, bioconjugate or molecule as described herein, for use in the manufacture of a medicament for:
treating, preventing or minimising progression of cancer in a subject;
inhibiting TGFβ activity in a subject with cancer;
reducing or inhibiting radiation-induced TGFβ activity in a subject receiving or requiring radiation treatment for cancer;
inhibiting or preventing cancer treatment-related fibrosis in a subject; and/or
enhancing or increasing the likelihood of success of treatment with an immune checkpoint inhibitor in a subject
   wherein the composition, bioconjugate or molecule comprises an antigen binding protein that binds to or specifically binds to an antigen expressed by the cancer and wherein the molecule, or bioconjugate, further comprises an ECD or ligand binding fragment of a TGFβR. Optionally, the molecule may be conjugated to a radionuclide.

In any embodiment, the medicament as described above, may be for use after the subject has received a prior treatment for the cancer, wherein the prior treatment is suspected of causing, or causes, an increase in TGFβ activity in the tumour microenvironment. The prior treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

In any embodiment, the medicament as described above, may be for use prior to the subject receiving a treatment for the cancer, wherein the treatment is suspected of causing, or causes, an increase in TGFβ activity in the tumour microenvironment. The treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

Optionally, the medicament may be for the treatment of a subject that may be receiving a concomitant treatment for the cancer, wherein the concomitant treatment is suspected of causing, or causes, an increase in TGFβ activity in the tumour microenvironment. The concomitant treatment for cancer may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy. Optionally, the medicament may be for administration of a subsequent treatment for the cancer, wherein the subsequent treatment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

In any embodiment of the invention, wherein the molecule comprises a tumour antigen binding protein that binds specifically to CAIX, the antigen binding protein preferably comprises:
FR1-CDR1-FR2-CDR2-FR3-CDR3-FR4 and FR1a-CDR1a-FR2a-CDR2a-FR3a-CDR3a-FR4a
   wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
   wherein the sequence of any of the complementarity determining regions have an amino acid sequence as described in Table 2.

Preferably, FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

In any embodiment, the tumour antigen binding protein comprises an HCDR1, HCDR2, HCDR3 of a heavy chain comprising the amino acid sequence as set forth in any of SEQ ID NOs: 52, 68, 84, 100 or 116; and an LCDR1, LCDR2 and LCDR3 of a light chain comprising the amino acid sequence as set forth in any of SEQ ID NOs: 132, 148, 164, 180, 196 or 212. It will be understood that the CDR boundaries and sequences can be determined by any suitable method known to the skilled person, including but not limited to the Kabat, Chothia or IMGT methods, as further described herein.

Preferably, the tumour antigen binding protein comprises an antigen binding domain that consists essentially of or consists of an amino acids sequence of (in order of N to C terminus or C to N terminus) SEQ ID NO: 52, 68, 84, 100 or 116; and a sequence as set forth in SEQ ID NO: 132, 148, 164, 180, 196 or 212.

Preferably, the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 49, 65, 81, 97 or 113, a CDR2 comprising a sequence at least about 80%, at least 85%, at least 90%, at least 92%, at least 95%, at least 97%, at least 99% identical to a sequence set in SEQ ID NO:50, 66, 82, 98 or 114, and a CDR3 comprising a sequence at least about 80%, at least 85%, at least 90%, at least 92%, at least 95%, at least 97%, at least 99% identical to a sequence set forth in SEQ ID NO: 51, 67, 83, 99 or 115;
(ii) a VH comprising a sequence at least about 95% or 96% or 97% or 98% or 99% identical to a sequence set forth in SEQ ID NO: 52, 68, 84, 100 or 116;
(iii) a VL comprising a CDR1 comprising a sequence at least about 80%, at least 85%, at least 90%, at least 92%, at least 95%, at least 97%, at least 99% identical to a sequence set forth in SEQ ID NO: 129, 145, 161, 177, 193, or 209, a CDR2 comprising a sequence at least about 80%, at least 85%, at least 90%, at least 92%, at least 95%, at least 97%, at least 99% identical to a sequence set forth in SEQ ID NO: 130, 146, 162, 178, 194 or 210 and a CDR3 comprising a sequence at least about 80%, at least 85%, at least 90%, at least 92%, at least 95%, at least 97%, at least 99% identical to a sequence set forth in SEQ ID NO: 131, 147, 163, 179, 195, or 211;
(iv) a VL comprising a sequence at least about 95% identical to a sequence set forth in SEQ ID NO:132, 148, 164, 180, 196 or 212;
(v) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115;
(vi) a VH comprising a sequence set forth in SEQ ID NO: 52, 68, 84, 100 or 116;
(vii) a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211;
(viii) a VL comprising a sequence set forth in SEQ ID NO: 132, 148, 164, 180, 196 or 212;
(ix) a VH comprising a CDR1 comprising a sequence set forth in SEQ ID NO: 49, 65, 81, 97 or 113, a CDR2 comprising a sequence set forth between in SEQ ID NO: 50, 66, 82, 98 or 114 and a CDR3 comprising a sequence set forth in SEQ ID NO: 51, 67, 83, 99 or 115; and a VL comprising a CDR1 comprising a sequence set SEQ ID NO: 129, 145, 161, 177, 193, or 209, a CDR2 comprising a sequence set forth in SEQ ID NO: 130, 146, 162, 178, 194, or 210 and a CDR3 comprising a sequence set forth in SEQ ID NO: 131, 147, 163, 179, 195 or 211; or
(x) a VH comprising a sequence set forth in SEQ ID NO: 52, 68, 84, 100 or 116 and a VL comprising a sequence set forth in SEQ ID NO: 132, 148, 164, 180, 196 or 212.

Preferably, the tumour antigen binding protein comprises a variable heavy chain comprising an amino acid sequence as set forth in SEQ ID NO: 52, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX.

Preferably, the tumour antigen binding protein comprises a variable heavy chain comprising an amino acid sequence as set forth in SEQ ID NO: 52, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX and while comprising the same CDR sequences as defined for the variable heavy chain of SEQ ID NO: 52.

Preferably, the tumour antigen binding protein comprises a variable light chain comprising an amino acid sequence as set forth in SEQ ID NO: 132, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX.

Preferably, the tumour antigen binding protein comprises a variable light chain comprising an amino acid sequence as set forth in SEQ ID NO: 132, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX and while comprising the same CDR sequences as defined for the variable light chain of SEQ ID NO: 132.

Preferably, the tumour antigen binding protein comprises a variable heavy chain comprising an amino acid sequence as set forth in SEQ ID NO: 52, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 132; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto; while retaining binding affinity for CAIX.

Preferably, the tumour antigen binding protein comprises a variable heavy chain comprising an amino acid sequence as set forth in SEQ ID NO: 52, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 132; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX and while comprising the same CDR sequences as defined for the variable heavy chain of SEQ ID NO: 52 and the variable light chain of SEQ ID NO: 132.

In any embodiment, the tumour antigen binding protein further comprises a constant region of the heavy chain that comprises the amino acid sequence as set forth in SEQ ID NO: 225, or a sequence at least 80%, 81%, 82%, 84%, 84%, 85%, 86%, 87%, 88%, 89%, 90%, 91%, 92%, 93%, 94%, 95% or 96% or 97% or 98% or 99% identical thereto

In any embodiment, the tumour antigen binding protein further comprises a constant region of the light chain that comprises the amino acid sequence as set forth in SEQ ID NO: 229, or a sequence at least 80%, 81%, 82%, 84%, 84%, 85%, 86%, 87%, 88%, 89%, 90%, 91%, 92%, 93%, 94%, 95% or 96% or 97% or 98% or 99% identical thereto.

In embodiment wherein the molecule of the invention comprises a tumour antigen binding protein that binds specifically to PSMA, the antigen binding protein preferably comprises:
FR1-CDR1-FR2-CDR2-FR3-CDR3-FR4 and FR1a-CDR1a-FR2a-CDR2a-FR3a-CDR3a-FR4a
   wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
   wherein the sequence of any of the complementarity determining regions have an amino acid sequence as described in Table 1.

Preferably, FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

In any embodiment, the tumour antigen binding protein comprises an HCDR1, HCDR2, HCDR3 of a heavy chain comprising the amino acid sequence as set forth in any of SEQ ID NOs: 4, 20 or 244; and an LCDR1, LCDR2 and LCDR3 of a light chain comprising the amino acid sequence as set forth in any of SEQ ID NOs: 36 or 245. It will be understood that the CDR boundaries and sequences can be determined by any suitable method known to the skilled person, including but not limited to the Kabat, Chothia or IMGT methods, as further described herein.

Preferably, the tumour antigen binding protein comprises an antigen binding domain that consists essentially of or consists of an amino acid sequence of (in order of N to C terminus or C to N terminus) SEQ ID NO: 4, 20 or 244 and 36 or 245.

Preferably, the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 1 or 17, a CDR2 comprising a sequence at least about 80%, at least 85%, at least 90%, at least 92%, at least 95%, at least 97%, at least 99% identical to a sequence set in SEQ ID NO: 2 or 18, and a CDR3 comprising a sequence at least about 80%, at least 85%, at least 90%, at least 92%, at least 95%, at least 97%, at least 99% identical to a sequence set forth in SEQ ID NO: 3 or 19;
(ii) a VH comprising a sequence at least about 95% or 96% or 97% or 98% or 99% identical to a sequence set forth in SEQ ID NO: 4, 20 or 244;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO:36 or 245;
(v) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19;
(vi) a VH comprising a sequence set forth in SEQ ID NO: 4, 20 or 244;
(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 45;
(viii) a VL comprising a sequence set forth in SEQ ID NO: 36 or 245;
(ix) a VH comprising a CDR1 comprising a sequence set forth in SEQ ID NO: 1 or 17, a CDR2 comprising a sequence set forth between in SEQ ID NO: 2 or 18 and a CDR3 comprising a sequence set forth in SEQ ID NO: 3 or 19; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 4, 20 or 244 and a VL comprising a sequence set forth in SEQ ID NO: 36 or 245.

Preferably, the tumour antigen binding protein comprises a variable heavy chain comprising an amino acid sequence as set forth in SEQ ID NO: 244, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PSMA.

Preferably, the tumour antigen binding protein comprises a variable heavy chain comprising an amino acid sequence as set forth in SEQ ID NO: 244, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PSMA and while comprising the same CDR sequences as defined for the variable heavy chain of SEQ ID NO: 244.

Preferably, the tumour antigen binding protein comprises a variable light chain comprising an amino acid sequence as set forth in SEQ ID NO: 245, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PSMA.

Preferably, the tumour antigen binding protein comprises a variable light chain comprising an amino acid sequence as set forth in SEQ ID NO: 245, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, which retains binding affinity for PSMA and while comprising the same CDR sequences as defined for the variable light chain of SEQ ID NO: 245.

Preferably, the tumour antigen binding protein comprises a variable heavy chain comprising an amino acid sequence as set forth in SEQ ID NO: 244, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 245; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto; while retaining binding affinity for PSMA.

Preferably, the tumour antigen binding protein comprises a variable heavy chain comprising an amino acid sequence as set forth in SEQ ID NO: 244, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 245; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PSMA and while comprising the same CDR sequences as defined for the variable heavy chain of SEQ ID NO: 244 and the variable light chain of SEQ ID NO: 245.

In any embodiment wherein the molecule of the invention comprises a tumour antigen binding protein that binds specifically to PDGFRα, the antigen binding protein preferably comprises:
FR1-CDR1-FR2-CDR2-FR3-CDR3-FR4 and FR1a-CDR1a-FR2a-CDR2a-FR3a-CDR3a-FR4a
   wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
   wherein the sequence of any of the complementarity determining regions have an amino acid sequence as described in Table 5.

Preferably, FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

In any embodiment, the tumour antigen binding protein comprises an HCDR1, HCDR2, HCDR3 of a heavy chain comprising the amino acid sequence as set forth in SEQ ID NO: 265 and an LCDR1, LCDR2 and LCDR3 of a light chain comprising the amino acid sequence as set forth in SEQ ID NO: 266. It will be understood that the CDR boundaries and sequences can be determined by any suitable method known to the skilled person, including but not limited to the Kabat, Chothia or IMGT methods, as further described herein.

Preferably, the tumour antigen binding protein comprises an antigen binding domain that consists essentially of or consists of an amino acid sequence of (in order of N to C terminus or C to N terminus) SEQ ID NO: 265 and/or 266.

Preferably, the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 267, a CDR2 comprising a sequence at least about 80%, at least 85%, at least 90%, at least 92%, at least 95%, at least 97%, at least 99% identical to a sequence set in SEQ ID NO: 268, and a CDR3 comprising a sequence at least about 80%, at least 85%, at least 90%, at least 92%, at least 95%, at least 97%, at least 99% identical to a sequence set forth in SEQ ID NO: 269;
(ii) a VH comprising a sequence at least about 95% or 96% or 97% or 98% or 99% identical to a sequence set forth in SEQ ID NO: 265;
(iii) a VL comprising a CDR1 comprising a sequence at least about 80%, at least 85%, at least 90%, at least 92%, at least 95%, at least 97%, at least 99% identical to a sequence set forth in SEQ ID NO: 270, a CDR2 comprising a sequence at least about 80%, at least 85%, at least 90%, at least 92%, at least 95%, at least 97%, at least 99% identical to a sequence set forth in SEQ ID NO: 271 and a CDR3 comprising a sequence at least about 80%, at least 85%, at least 90%, at least 92%, at least 95%, at least 97%, at least 99% identical to a sequence set forth in SEQ ID NO: 272;
(iv) a VL comprising a sequence at least about 95% identical to a sequence set forth in SEQ ID NO: 266;
(v) a VH comprising a CDR1 comprising a sequence set forth in SEQ ID NO: 267, a CDR2 comprising a sequence set forth between in SEQ ID NO: 268 and a CDR3 comprising a sequence set forth in SEQ ID NO: 269;
(vi) a VH comprising a sequence set forth in SEQ ID NO: 265,
(vii) a VL comprising a CDR1 comprising a sequence set SEQ ID NO: 270, a CDR2 comprising a sequence set forth in SEQ ID NO: 271 and a CDR3 comprising a sequence set forth in SEQ ID NO: 272;
(viii) a VL comprising a sequence set forth in SEQ ID NO: 266;
(ix) a VH comprising a CDR1 comprising a sequence set forth in SEQ ID NO: 267, a CDR2 comprising a sequence set forth between in SEQ ID NO: 268 and a CDR3 comprising a sequence set forth in SEQ ID NO: 269; and a VL comprising a CDR1 comprising a sequence set SEQ ID NO: 270, a CDR2 comprising a sequence set forth in SEQ ID NO: 271 and a CDR3 comprising a sequence set forth in SEQ ID NO: 272; or
(x) a VH comprising a sequence set forth in SEQ ID NO: 265 and a VL comprising a sequence set forth in SEQ ID NO: 266.

Preferably, the tumour antigen binding protein comprises a variable heavy chain comprising an amino acid sequence as set forth in SEQ ID NO: 265, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PDGFRα.

Preferably, the tumour antigen binding protein comprises a variable heavy chain comprising an amino acid sequence as set forth in SEQ ID NO: 265, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PDGFRα and while comprising the same CDR sequences as defined for the variable heavy chain of SEQ ID NO: 265.

Preferably, the tumour antigen binding protein comprises a variable light chain comprising an amino acid sequence as set forth in SEQ ID NO: 266, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PDGFRα.

Preferably, the tumour antigen binding protein comprises a variable light chain comprising an amino acid sequence as set forth in SEQ ID NO: 266, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, which retains binding affinity for PDGFRα and while comprising the same CDR sequences as defined for the variable light chain of SEQ ID NO: 266.

Preferably, the tumour antigen binding protein comprises a variable heavy chain comprising an amino acid sequence as set forth in SEQ ID NO: 265, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 266; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto; while retaining binding affinity for PDGFRα.

Preferably, the tumour antigen binding protein comprises a variable heavy chain comprising an amino acid sequence as set forth in SEQ ID NO: 265, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 266; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PDGFRα and while comprising the same CDR sequences as defined for the variable heavy chain of SEQ ID NO: 265 and the variable light chain of SEQ ID NO: 266.

In any embodiment wherein the molecule of the invention comprises a tumour antigen binding protein that binds specifically to La/SSB, the antigen binding protein preferably comprises:
FR1-CDR1-FR2-CDR2-FR3-CDR3-FR4 and FR1a-CDR1a-FR2a-CDR2a-FR3a-CDR3a-FR4a
   wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
   wherein the sequence of any of the complementarity determining regions have an amino acid sequence as described in Table 6.

Preferably, FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

In any embodiment, the tumour antigen binding protein comprises an HCDR1, HCDR2, HCDR3 of a heavy chain comprising the amino acid sequence as set forth in SEQ ID NO: 303 and an LCDR1, LCDR2 and LCDR3 of a light chain comprising the amino acid sequence as set forth in SEQ ID NO: 304. It will be understood that the CDR boundaries and sequences can be determined by any suitable method known to the skilled person, including but not limited to the Kabat, Chothia or IMGT methods, as further described herein.

Preferably, the tumour antigen binding protein comprises an antigen binding domain that consists essentially of or consists of an amino acid sequence of (in order of N to C terminus or C to N terminus) SEQ ID NO: 303 and/or 304.

Preferably, the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 305, a CDR2 comprising a sequence at least about 80%, at least 85%, at least 90%, at least 92%, at least 95%, at least 97%, at least 99% identical to a sequence set in SEQ ID NO: 306, and a CDR3 comprising a sequence at least about 80%, at least 85%, at least 90%, at least 92%, at least 95%, at least 97%, at least 99% identical to a sequence set forth in SEQ ID NO: 307;
(ii) a VH comprising a sequence at least about 95% or 96% or 97% or 98% or 99% identical to a sequence set forth in SEQ ID NO: 303;
(iii) a VL comprising a CDR1 comprising a sequence at least about 80%, at least 85%, at least 90%, at least 92%, at least 95%, at least 97%, at least 99% identical to a sequence set forth in SEQ ID NO: 308, a CDR2 comprising a sequence at least about 80%, at least 85%, at least 90%, at least 92%, at least 95%, at least 97%, at least 99% identical to a sequence set forth in SEQ ID NO: 309 and a CDR3 comprising a sequence at least about 80%, at least 85%, at least 90%, at least 92%, at least 95%, at least 97%, at least 99% identical to a sequence set forth in SEQ ID NO: 310;
(iv) a VL comprising a sequence at least about 95% identical to a sequence set forth in SEQ ID NO: 304;
(v) a VH comprising a CDR1 comprising a sequence set forth in SEQ ID NO: 305, a CDR2 comprising a sequence set forth between in SEQ ID NO: 306 and a CDR3 comprising a sequence set forth in SEQ ID NO: 307;
(vi) a VH comprising a sequence set forth in SEQ ID NO: 303,
(vii) a VL comprising a CDR1 comprising a sequence set SEQ ID NO: 308, a CDR2 comprising a sequence set forth in SEQ ID NO: 309 and a CDR3 comprising a sequence set forth in SEQ ID NO: 310;
(viii) a VL comprising a sequence set forth in SEQ ID NO: 304;
(ix) a VH comprising a CDR1 comprising a sequence set forth in SEQ ID NO: 305, a CDR2 comprising a sequence set forth between in SEQ ID NO: 306 and a CDR3 comprising a sequence set forth in SEQ ID NO: 307; and a VL comprising a CDR1 comprising a sequence set SEQ ID NO: 308, a CDR2 comprising a sequence set forth in SEQ ID NO: 309 and a CDR3 comprising a sequence set forth in SEQ ID NO: 310; or
(x) a VH comprising a sequence set forth in SEQ ID NO: 303 and a VL comprising a sequence set forth in SEQ ID NO: 304.

Preferably, the tumour antigen binding protein comprises a variable heavy chain comprising an amino acid sequence as set forth in SEQ ID NO: 303, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for La/SSB.

Preferably, the tumour antigen binding protein comprises a variable heavy chain comprising an amino acid sequence as set forth in SEQ ID NO: 303, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for La/SSB and while comprising the same CDR sequences as defined for the variable heavy chain of SEQ ID NO: 303.

Preferably, the tumour antigen binding protein comprises a variable light chain comprising an amino acid sequence as set forth in SEQ ID NO: 304, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for La/SSB.

Preferably, the tumour antigen binding protein comprises a variable light chain comprising an amino acid sequence as set forth in SEQ ID NO: 304, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, which retains binding affinity for La/SSB and while comprising the same CDR sequences as defined for the variable light chain of SEQ ID NO: 304.

Preferably, the tumour antigen binding protein comprises a variable heavy chain comprising an amino acid sequence as set forth in SEQ ID NO: 303, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 304; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto; while retaining binding affinity for La/SSB.

Preferably, the tumour antigen binding protein comprises a variable heavy chain comprising an amino acid sequence as set forth in SEQ ID NO: 303, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, and a variable light chain comprising an amino acid sequence as set forth in SEQ ID NO: 304; or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for La/SSB and while comprising the same CDR sequences as defined for the variable heavy chain of SEQ ID NO: 303 and the variable light chain of SEQ ID NO: 304.

An antigen binding protein as described herein may comprise a human constant region, e.g., an IgG constant region, such as an IgG1, IgG2, IgG3 or IgG4 constant region or mixtures thereof. In the case of an antibody or protein comprising a VH and a VL, the VH can be linked to a heavy chain constant region and the VL can be linked to a light chain constant region.

In one example, an antigen binding protein as described herein comprises a constant region of an IgG4 antibody or a stabilized constant region of an IgG4 antibody. In one example, the protein or antibody comprises an IgG4 constant region with a proline at position 241 (according to the numbering system of Kabat (Kabat et al., Sequences of Proteins of Immunological Interest Washington DC United States Department of Health and Human Services, 1987 and/or 1991)).

In one example, an antigen binding protein as described herein or a composition of an antigen binding protein as described herein, comprises a heavy chain constant region, comprising a stabilized heavy chain constant region, comprising a mixture of sequences fully or partially with or without the C-terminal lysine residue.

In one example, an antigen binding protein comprises a VH disclosed herein linked or fused to an IgG4 constant region or stabilized IgG4 constant region (e.g., as discussed above) and the VL is linked to or fused to a kappa light chain constant region.

An antigen binding protein or molecule as described herein may be purified, substantially purified, isolated and/or recombinant.

In any embodiment, the tumour antigen binding protein further comprises a constant region of the heavy chain that comprises the amino acid sequence as set forth in SEQ ID NO: 225, or a sequence at least 80%, 81%, 82%, 84%, 84%, 85%, 86%, 87%, 88%, 89%, 90%, 91%, 92%, 93%, 94%, 95% or 96% or 97% or 98% or 99% identical thereto. In any embodiment, the heavy chain constant region may comprise one or more amino acid substitutions for stabilising the linkage to the ECD or ligand binding domain of the TGFβR. Such substitutions are further described herein and are also known to the skilled person in the art, in relation to stabilisation of fusion proteins.

In any embodiment, the tumour antigen binding protein further comprises a constant region of the light chain that comprises the amino acid sequence as set forth in SEQ ID NO: 229, or a sequence at least 80%, 81%, 82%, 84%, 84%, 85%, 86%, 87%, 88%, 89%, 90%, 91%, 92%, 93%, 94%, 95% or 96% or 97% or 98% or 99% identical thereto.

In any embodiment, the ECD or ligand binding domain of the TGFβR may comprise or consist of an amino acid sequence as set forth in any one of SEQ ID NOs: 246 to 254, or as further herein described including in Table 4. In a preferred embodiment, the molecule of the invention may comprise the amino acid sequence as set forth in SEQ ID NO: 249, or 321, or a sequence at least about 80%, 81%, 82%, 84%, 84%, 85%, 86%, 87%, 88%, 89%, 90%, 91%, 92%, 93%, 94%, 95% or 96% or 97% or 98% or 99% identical thereto, which retains the ability to bind to TGFβ.

In embodiments of the invention, the molecule is in the form of an antibody, wherein the antibody comprises a heavy chain joined to the ECD or ligand binding domain of the TGFβR, and a light chain. Accordingly the invention provides for fusion proteins comprising the sequence of the heavy chain of an antibody, as herein described, and an ECD or ligand binding domain of the TGFβR. In further embodiments, the molecule is in the form of an antibody, wherein the antibody comprises a light chain joined to the ECD or ligand binding domain of the TGFβR, and a heavy chain. Accordingly the invention provides for fusion proteins comprising the sequence of the light chain of an antibody, as herein described, and an ECD or ligand binding domain of the TGFβR.

In particularly preferred embodiments of the invention, the molecule is in the form of an antibody, wherein the antibody comprises a heavy chain joined to the ECD or ligand binding domain of the TGFβR, and a light chain. Exemplary heavy and light chain pairs are set forth herein in Table 5. In a preferred embodiment, the molecule comprises an antibody for binding CAIX and comprises a heavy chain comprising the amino acid sequence as set forth in SEQ ID NO: 257 or 258 and a light chain comprising the amino acid sequence as set forth in SEQ ID NO: 259. In a further preferred embodiment, the molecule comprises an antibody for binding PSMA and comprises a heavy chain comprising the amino acid sequence as set forth in SEQ ID NO: 262 or 263 and a light chain comprising the amino acid sequence as set forth in SEQ ID NO: 264.

The present invention additionally comprises a kit comprising one or more of the following:
(i) an molecule of the invention or expression construct(s) encoding same;
(iii) a pharmaceutical composition of the invention.

The kit may additionally comprise a pharmaceutically acceptable carrier.

In certain embodiments, the kit may comprise one or more additional therapeutic agents for administration to the subject prior to or following treatment with a molecule or composition of the invention, as described herein. Optionally, the kit may comprise written instructions for use of the components of the kit.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF DRAWINGS

FIG. 1. Representative SDS page (A) and SEC-HPLC (B) from an anti-CAIX TGFβ fusion protein of the invention (TLX250 trap).

FIG. 2. Anti-CAIX TGFβ fusion protein specifically binds CAIX-expressing cells. (A) Representative histograms from CT26-hCAIX cells incubated with anti-CAIX TGFβ fusion protein (TLX250 trap) or anti-PSMA TGFβ fusion protein (TLX591 trap), followed by incubation with an anti-human IgG. Concentration of the primary antibody was 0.1235 ug/ml. (B) gMFI of the signal from CT26-hCAIX cells treated as in (A) across a dose range. A single concentration (open circle) of the parent antibody Girentuximab is shown for reference.

FIG. 3. Anti-PSMA TGFβ fusion protein specifically binds PSMA-expressing cells. (A) Representative histograms from LnCap cells incubated with the anti-PSMA TGFβ fusion protein (TLX591 trap) or anti-CAIX TGFβ fusion protein (TLX250 trap), followed by incubation with an anti-human IgG. Concentration of the primary antibody was 90 ug/ml. (B) gMFI of the signal from LnCap cells treated as in (A) across a dose range. A single concentration (open circle) of the parent antibody HuJ591 is shown for reference.

FIG. 4. Anti-CAIX TGFβ fusion protein binds all 3 isoforms of TGF-beta in vitro. For TGF-beta1 and TGF-beta3, TGF-beta was coated on plates and incubated with anti-CAIX TGFβ fusion protein (TLX250 trap) and binding was determined by ELISA using an anti-human IgG. For TGF-beta2, antibodies were coated on plates, and incubated with TGF-beta2, which was detected by ELISA using an anti-TGF-beta2 detection antibody. The parent antibody (Girentuximab) was used as a negative control.

FIG. 5. Representative SDS page (A) and SEC-HPLC (B) from an anti-PSMA TGFβ fusion protein (TLX591 trap).

FIG. 6. Anti-PSMA TGFβ fusion protein binds all 3 isoforms of TGF-beta in vitro. For TGF-beta1 and TGF-beta3, TGF-beta was coated on plates and incubated with anti-PSMA TGFβ fusion protein (TLX591 trap) and binding was determined by ELISA using an anti-human IgG. For TGF-beta2, antibodies were coated on plates, and incubated with TGF-beta2, which was detected by ELISA using an anti-TGF-beta2 detection antibody. The parent antibody (HuJ591) was used as a negative control.

FIG. 7. A radiolabeled anti-PSMA TGFβ fusion protein binds TGFβ1 with high affinity in vitro. Plates were coated with TGFβ1, then incubated with varying concentrations of 89Zr radiolabeled antibodies. After washing, radioactivity in wells was determined. Anti-TGFb refers to the positive control antibody Fresolimumab and HuJ591 refers to the negative control parent antibody for the anti-PSMA TGFβ fusion protein (TLX591 trap).

FIG. 8. Anti-PSMA TGFβ fusion protein sequesters TGF-beta in the Tumor Microenvironment. RM1-hPSMA tumour bearing animals were treated according to the timeline in (A). Control or anti-PSMA TGFβ fusion protein (TLX591 trap) pre-treated animals were imaged for TGF-beta using an 89Zr-labeled anti-TGFβ antibody (Fresolimumab). TLX591 trap pre-treated animals had a substantially reduced PET signal indicating the TLX591 trap had sequestered TGFβ from 89Zr Fresolimumab (B). % ID/ml=% injected dose per ml. n=5 animals per group

FIG. 9. Anti-PSMA TGFβ fusion protein sequesters TGF-beta systemically. RM1-hPSMA tumour bearing animals were treated according to the timeline in (A). Graph in (B) shows serum concentrations of TGF-beta1 as measured by ELISA. Animals treated with the anti-PSMA TGFβ fusion protein (TLX591 trap) or a positive control anti-TGFβ mAb had substantially reduced serum levels of TGF-beta1. EBRT=10 Gy External Beam RadioTherapy. No Tx=No Treatment.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

The present invention seeks to overcome at least some of the limitations of existing treatments for cancer, and of other conditions, where TGFβ expression is prevalent or wherein treatment results in induction of TGFβ expression, particularly in the tumour microenvironment.

Increased expression of TGFβ is a key driver of the wound-healing response to various cancer treatments, such as, but not limited to radiation therapy, chemotherapy and surgery. TGFβ can promote activity of suppressive regulatory cells that restrain anti-tumour T cells. In addition, increased TGFβ expression can drive fibrosis, which may result in a physical barrier to anti-cancer T cells entering the tumour. TGF-beta not only limits T cell responses, but also promotes DNA damage responses and EMT in tumour cells, making them radioresistant.

Accordingly, the present invention seeks to inhibit or reduce baseline and treatment-induced TGFβ activity, specifically in the tumour microenvironment. Without wishing to be bound by theory, the inventors consider that this approach may facilitate reduction in tumour fibrosis and enhancement of anti-cancer immunity. The present invention thus provides tumour antigen binding proteins (eg antibodies), fused to a “molecular trap” in the form of a TGFβ binding domain for binding to TGFβ, thereby preventing TGF-beta from interacting with and signalling through its normal (ie inhibiting TGFβ activity). As explained further herein, the TGFβ binding domain is preferably in the form of a ligand binding domain, or extracellular domain of a TGFβ receptor. Such molecules of the invention may be utilised following any treatment for cancer that results in the increased expression of TGFβ, such as external beam radiation, molecular targeted radiation, surgery, chemotherapy, immunotherapy and treatment with an immune checkpoint inhibitor. Because the molecules of the invention comprise antigen binding domains for specifically targeting the molecular trap to the site of the tumour cells, it is thought that the molecules may maximise reduction of TGFβ activity in the tumour microenvironment.

In certain embodiments of the invention, the molecules of the invention may also be conjugated to a radioisotope. Thus, a particular advantage of one approach of the present invention, is the ability to physically link the “molecular trap” (for binding TGFβ), and the source of radiation, to be delivered to tumour microenvironment via the tumour antigen binding protein of the molecule. Thus, in preferred embodiments of the invention, the molecules and compositions of the invention provide for simultaneous tumour-targeted radiation, and mitigation of a key suppressive compensatory mechanism that dampens its activity.

Further still, the approach of the present invention may assist in preparing the tumour microenvironment for subsequent treatment, such as with immune checkpoint inhibitors. For example, it is believed that increased TGFβ activity, which may occur after radiation or other cancer treatment, may limit the response to treatment with a checkpoint inhibitor, thereby at least in part contributing to immune-exclusion. Thus, in further embodiments, the present invention seeks to improve the likelihood of success of downstream treatments for cancer following initial treatments which may result in increased TGFβ signalling.

### GENERAL

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The description and definitions of variable regions and parts thereof, immunoglobulins, antibodies and fragments thereof herein may be further clarified by the discussion in Kabat Sequences of Proteins of Immunological Interest, National Institutes of Health, Bethesda, Md., 1987 and 1991, Bork et al., J Mol. Biol. 242, 309-320, 1994, Chothia and Lesk J. Mol Biol. 196:901-917, 1987, Chothia et al. Nature 342, 877-883, 1989 and/or or Al-Lazikani et al., J Mol Biol 273, 927-948, 1997.

The term “and/or”, e.g., “X and/or Y” shall be understood to mean either “X and Y” or “X or Y” and shall be taken to provide explicit support for both meanings or for either meaning.

As used herein, at least 70% sequence identity should be understood to provide basis for at least 70%, at least 71%, at least 72%, at least 73%, at least 74%, at least 75%, at least 76%, at least 77%, at least 78%, at least 79% and at least 80% identity. At least 80% sequence identity should be understood to provide basis for at least 80%, at least 81%, at least 82%, at least 83%, at least 84%, at least 85%, at least 86%, at least 87%, at least 88%, at least 89% and at least 90% identity. At least 90% sequence identity should be understood to provide basis for at least 90%, at least 91%, at least 92%, at least 93%, at least 94%, at least 95%, at least 96%, at least 97%, at least 98%, and at least 99% identity.

As used herein the term “derived from” shall be taken to indicate that a specified integer may be obtained from a particular source albeit not necessarily directly from that source.

### Selected Definitions

As used herein, the term “antigen binding protein” is used interchangeably with “antigen binding domain” and shall be taken to mean a region of an antibody that is capable of specifically binding to an antigen, i.e., a VH or a VL or an Fv comprising both a VH and a VL. The antigen binding domain need not be in the context of an entire antibody, e.g., it can be in isolation (e.g., a domain antibody) or in another form, e.g., as described herein, such as a scFv.

For the purposes for the present disclosure, the term “antibody” includes a protein capable of specifically binding to one or a few closely related antigens by virtue of an antigen binding domain contained within a Fv. This term includes four chain antibodies (e.g., two light chains and two heavy chains), recombinant or modified antibodies (e.g., chimeric antibodies, humanized antibodies, human antibodies, CDR-grafted antibodies, primatized antibodies, de-immunized antibodies, synhumanized antibodies, half-antibodies, bispecific antibodies). An antibody generally comprises constant domains, which can be arranged into a constant region or constant fragment or fragment crystallizable (Fc). Exemplary forms of antibodies comprise a four-chain structure as their basic unit. Full-length antibodies comprise two heavy chains (~50 to 70 kD) covalently linked and two light chains (~23 kDa each). A light chain generally comprises a variable region (if present) and a constant domain and in mammals is either a κ light chain or a λ light chain. A heavy chain generally comprises a variable region and one or two constant domain(s) linked by a hinge region to additional constant domain(s). Heavy chains of mammals are of one of the following types α, δ, ε, γ, or μ. Each light chain is also covalently linked to one of the heavy chains. For example, the two heavy chains and the heavy and light chains are held together by inter-chain disulfide bonds and by non-covalent interactions. The number of inter-chain disulfide bonds can vary among different types of antibodies. Each chain has an N-terminal variable region (VH or VL wherein each are ~110 amino acids in length) and one or more constant domains at the C-terminus. The constant domain of the light chain (CL which is ~110 amino acids in length) is aligned with and disulfide bonded to the first constant domain of the heavy chain (CH1 which is 330 to 440 amino acids in length). The light chain variable region is aligned with the variable region of the heavy chain. The antibody heavy chain can comprise 2 or more additional CH domains (such as, CH2, CH3 and the like) and can comprise a hinge region between the CH1 and CH2 constant domains. Antibodies can be of any type (e.g., IgG, IgE, IgM, IgD, IgA, and IgY), class (e.g., IgG1, IgG2, IgG3, IgG4, IgA1 and IgA2) or subclass. In one example, the antibody is a murine (mouse or rat) antibody or a primate (such as, human) antibody. In one example the antibody heavy chain is missing a C-terminal lysine residue. In one example, the antibody is humanized, synhumanized, chimeric, CDR-grafted or deimmunized.

The terms “full-length antibody”, “intact antibody” or “whole antibody” are used interchangeably to refer to an antibody in its substantially intact form, as opposed to an antigen binding fragment of an antibody. Specifically, whole antibodies include those with heavy and light chains including an Fc region. The constant domains may be wild-type sequence constant domains (e.g., human wild-type sequence constant domains) or amino acid sequence variants thereof.

As used herein, “variable region” refers to the portions of the light and/or heavy chains of an antibody as defined herein that is capable of specifically binding to an antigen and, includes amino acid sequences of complementarity determining regions (CDRs); i.e., CDR1, CDR2, and CDR3, and framework regions (FRs). For example, the variable region comprises three or four FRs (e.g., FR1, FR2, FR3 and optionally FR4) together with three CDRs. VH refers to the variable region of the heavy chain. VL refers to the variable region of the light chain.

As used herein, the term “complementarity determining regions” (syn. CDRs; i.e., CDR1, CDR2, and CDR3) refers to the amino acid residues of an antibody variable region the presence of which are major contributors to specific antigen binding. Each variable region domain (VH or VL) typically has three CDRs identified as CDR1, CDR2 and CDR3. The CDRs of VH are also referred to herein as CDR H1, CDR H2 and CDR H3, respectively, wherein CDR H1 corresponds to CDR 1 of VH, CDR H2 corresponds to CDR 2 of VH and CDR H3 corresponds to CDR 3 of VH. Likewise, the CDRs of VL are referred to herein as CDR L1, CDR L2 and CDR L3, respectively, wherein CDR L1 corresponds to CDR 1 of VL, CDR L2 corresponds to CDR 2 of VL and CDR L3 corresponds to CDR 3 of VL. In one example, the amino acid positions assigned to CDRs and FRs are defined according to Kabat Sequences of Proteins of Immunological Interest, National Institutes of Health, Bethesda, Md., 1987 and 1991 (also referred to herein as “the Kabat numbering system”). In another example, the amino acid positions assigned to CDRs and FRs are defined according to the Enhanced Chothia Numbering Scheme (http://www.bioinfo.org.uk/mdex.html). The present invention is not limited to FRs and CDRs as defined by the Kabat numbering system, but includes all numbering systems, including the canonical numbering system or of Chothia and Lesk J. Mol. Biol. 196: 901-917, 1987; Chothia et al., Nature 342: 877-883, 1989; and/or Al-Lazikani et al., J. Mol. Biol. 273: 927-948, 1997; the numbering system of Honnegher and Plükthun J. Mol. Biol. 309: 657-670, 2001; or the IMGT system discussed in Giudicelli et al., Nucleic Acids Res. 25: 206-211 1997. In one example, the CDRs are defined according to the Kabat numbering system. Optionally, heavy chain CDR2 according to the Kabat numbering system does not comprise the five C-terminal amino acids listed herein or any one or more of those amino acids are substituted with another naturally-occurring amino acid. In this regard, Padlan et al., FASEB J., 9: 133-139, 1995 established that the five C-terminal amino acids of heavy chain CDR2 are not generally involved in antigen binding.

“Framework regions” (FRs) are those variable region residues other than the CDR residues. The FRs of VH are also referred to herein as FR H1, FR H2, FR H3 and FR H4, respectively, wherein FR H1 corresponds to FR 1 of VH, FR H2 corresponds to FR 2 of VH, FR H3 corresponds to FR 3 of VH and FR H4 corresponds to FR 4 of VH. Likewise, the FRs of VL are referred to herein as FR L1, FR L2, FR L3 and FR L4, respectively, wherein FR L1 corresponds to FR 1 of VL, FR L2 corresponds to FR 2 of VL, FR L3 corresponds to FR 3 of VL and FR L4 corresponds to FR 4 of VL.

As used herein, the term “Fv” shall be taken to mean any protein, whether comprised of multiple polypeptides or a single polypeptide, in which a VL and a VH associate and form a complex having an antigen binding domain, i.e., capable of specifically binding to an antigen. The VH and the VL which form the antigen binding domain can be in a single polypeptide chain or in different polypeptide chains. Furthermore, an Fv of the invention (as well as any protein of the invention) may have multiple antigen binding domains which may or may not bind the same antigen. This term shall be understood to encompass fragments directly derived from an antibody as well as proteins corresponding to such a fragment produced using recombinant means. In some examples, the VH is not linked to a heavy chain constant domain (CH) 1 and/or the VL is not linked to a light chain constant domain (CL). Exemplary Fv containing polypeptides or proteins include a Fab fragment, a Fab′ fragment, a F(ab′) fragment, a scFv, a diabody, a triabody, a tetrabody or higher order complex, or any of the foregoing linked to a constant region or domain thereof, e.g., CH2 or CH3 domain, e.g., a minibody. A “Fab fragment” consists of a monovalent antigen-binding fragment of an immunoglobulin, and can be produced by digestion of a whole antibody with the enzyme papain, to yield a fragment consisting of an intact light chain and a portion of a heavy chain or can be produced using recombinant means. A “Fab′ fragment” of an antibody can be obtained by treating a whole antibody with pepsin, followed by reduction, to yield a molecule consisting of an intact light chain and a portion of a heavy chain comprising a VH and a single constant domain. Two Fab′ fragments are obtained per antibody treated in this manner. A Fab′ fragment can also be produced by recombinant means. A “F(ab′)2 fragment” of an antibody consists of a dimer of two Fab′ fragments held together by two disulfide bonds, and is obtained by treating a whole antibody molecule with the enzyme pepsin, without subsequent reduction. A “Fab2” fragment is a recombinant fragment comprising two Fab fragments linked using, for example a leucine zipper or a CH3 domain. A “single chain Fv” or “scFv” is a recombinant molecule containing the variable region fragment (Fv) of an antibody in which the variable region of the light chain and the variable region of the heavy chain are covalently linked by a suitable, flexible polypeptide linker.

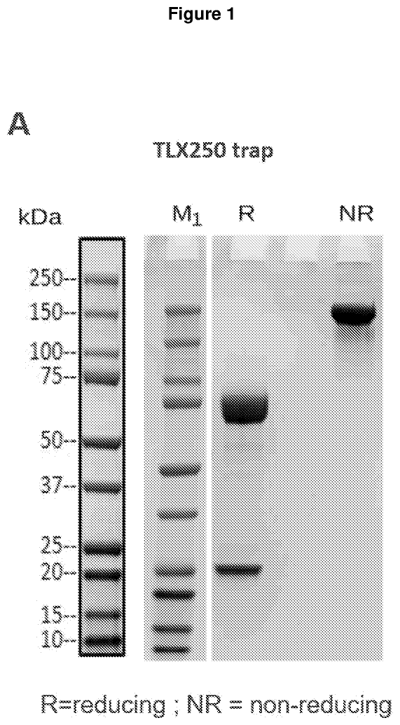

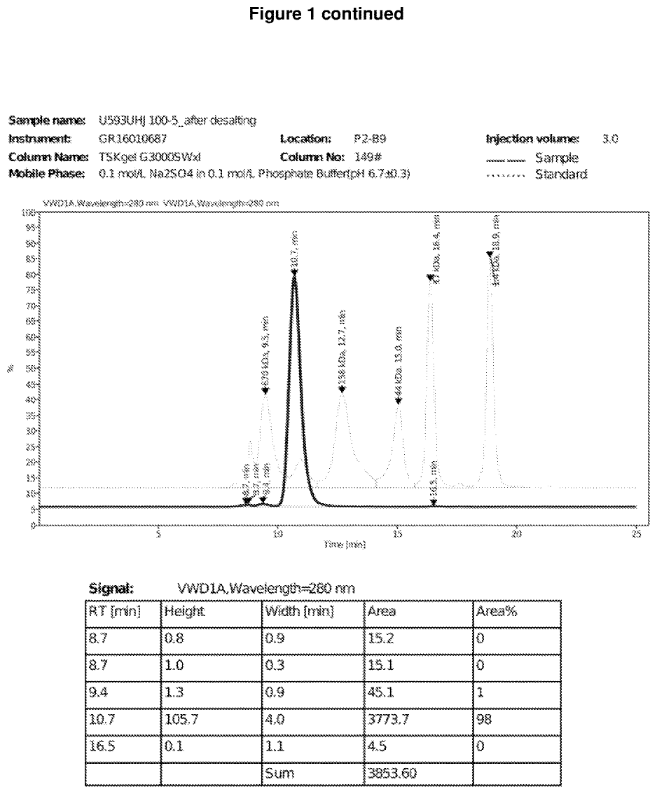

The term “Fc region”, sometimes referred to as “Fc” or “Fc domain”, as used herein refers the portion of an IgG molecule that correlates to a crystallizable fragment obtained by papain digestion of an IgG molecule. The Fc region consists of the C-terminal half of the two heavy chains of an IgG molecule that are linked by disulfide bonds. It has no antigen binding activity but contains the carbohydrate moiety and the binding sites for complement and Fc receptors, including the FcRn receptor. The Fc region contains the entire second constant domain CH2 (residues 231-340 of human IgG1, according to the EU Index numbering system, also defined as residues 244 to 360 in the Kabat system) and the third constant domain CH3 (residues 341-447 EU Index/361-478 Kabat) (e.g., see SEQ ID NO 1 of WO2015175874 or FIG. 1C for the sequence of CH2 and SEQ ID NO:2; FIG. 1D for the sequence of CH3, incorporated herein by reference; see also http://www.imgt.org/IMGTScientificChart/Numbering/Hu_IGHGnber.html #refs for a comparison of the numbering conventions used for various residues in the Fc region of immunoglobulins).

As used herein, the “EU index” or “EU numbering scheme” refers to the numbering of the EU antibody (Edelman et al., 1969, Proc Natl Acad Sci USA 63:78-85, hereby entirely incorporated by reference.) As used herein, the “Kabat system” refers to the Kabat Sequences of Proteins of Immunological Interest, National Institutes of Health, Bethesda, Md., 1987 and 1991. The skilled person will be able to readily determine whether a given amino acid sequence is numbered according to either EU or Kabat systems.

The term “isolated protein” or “isolated polypeptide” is a protein or polypeptide that by virtue of its origin or source of derivation is not associated with naturally-associated components that accompany it in its native state; is substantially free of other proteins from the same source. A protein may be rendered substantially free of naturally associated components or substantially purified by isolation, using protein purification techniques known in the art. By “substantially purified” is meant the protein is substantially free of contaminating agents, e.g., at least about 70% or 75% or 80% or 85% or 90% or 95% or 96% or 97% or 98% or 99% free of contaminating agents.

The term “recombinant” shall be understood to mean the product of artificial genetic recombination. Accordingly, in the context of a recombinant protein comprising an antibody antigen binding domain, this term does not encompass an antibody naturally-occurring within a subject's body that is the product of natural recombination that occurs during B cell maturation. However, if such an antibody is isolated, it is to be considered an isolated protein comprising an antibody antigen binding domain. Similarly, if nucleic acid encoding the protein is isolated and expressed using recombinant means, the resulting protein is a recombinant protein comprising an antibody antigen binding domain. A recombinant protein also encompasses a protein expressed by artificial recombinant means when it is within a cell, tissue or subject, e.g., in which it is expressed.

The term “protein” shall be taken to include a single polypeptide chain, i.e., a series of contiguous amino acids linked by peptide bonds or a series of polypeptide chains covalently or non-covalently linked to one another (i.e., a polypeptide complex). For example, the series of polypeptide chains can be covalently linked using a suitable chemical or a disulphide bond. Examples of non-covalent bonds include hydrogen bonds, ionic bonds, Van der Waals forces, and hydrophobic interactions.

The term “polypeptide” or “polypeptide chain” will be understood from the foregoing paragraph to mean a series of contiguous amino acids linked by peptide bonds.

As used herein, the term “binds” in reference to the interaction of an antigen binding protein or an antigen binding domain thereof with an antigen means that the interaction is dependent upon the presence of a particular structure (e.g., an antigenic determinant or epitope) on the antigen. For example, an antibody recognizes and binds to a specific protein structure rather than to proteins generally. If an antibody binds to epitope “A”, the presence of a molecule containing epitope “A” (or free, unlabelled “A”), in a reaction containing labeled “A” and the protein, will reduce the amount of labelled “A” bound to the antibody.

As used herein, the term “specifically binds” or “binds specifically” shall be taken to mean that an antigen binding protein of the invention reacts or associates more frequently, more rapidly, with greater duration and/or with greater affinity with a particular antigen or cell expressing same than it does with alternative antigens or cells. For example, an antigen binding protein binds to a specified tumour associated or tumour antigen, with materially greater affinity (e.g., 1.5 fold or 2 fold or 5 fold or 10 fold or 20 fold or 40 fold or 60 fold or 80 fold to 100 fold or 150 fold or 200 fold) than it does to other antigens.

As used herein, the term “epitope” (syn. “antigenic determinant”) shall be understood to mean a region of a cell surface protein (such as PSMA, CAIX PDGFRα and LA/SSB) to which an antigen binding protein comprising an antigen binding domain of an antibody binds.

As used herein, the term “condition” refers to a disruption of or interference with normal function, and is not to be limited to any specific condition, and will include diseases or disorders.

As used herein, the terms “preventing”, “prevent” or “prevention” include administering an antigen binding protein of the invention to thereby stop or hinder the development of at least one symptom of a condition. This term also encompasses treatment of a subject in remission to prevent or hinder relapse.

As used herein, the terms “treating”, “treat” or “treatment” include administering an antigen binding protein described herein to thereby reduce or eliminate at least one symptom of a specified disease or condition.

As used herein, the term “subject” shall be taken to mean any animal including humans, for example a mammal. Exemplary subjects include but are not limited to humans and non-human primates. For example, the subject is a human.

As used herein “tumour-associated antigen” (TAA) refers to an antigen that is expressed by cancer cells. A TAA associated antigen is not unique to a tumour cell and instead is also expressed on a normal cell under conditions that fail to induce a state of immunologic tolerance to the antigen. The expression of the antigen on the tumour may occur under conditions that enable the immune system to respond to the antigen. TAAs may be antigens that are expressed on normal cells during foetal development when the immune system is immature and unable to respond or they may be antigens that are normally present at extremely low levels on normal cells but which are expressed at much higher levels on tumour cells. Cytotoxic T lymphocytes that recognise these antigens may be able to destroy the tumour cells before they proliferate or metastasise. Tumour antigens may also be on the surface of the tumour in the form of, for example, a mutated receptor, in which case they may be recognised by B cells.

### Antibody Production

Preferably, an antigen binding protein described herein according to any example is recombinant.

In the case of a recombinant protein, nucleic acid encoding same can be cloned into expression constructs or vectors, which are then transfected into host cells, such as E. coli cells, yeast cells, insect cells, or mammalian cells, such as simian COS cells, Chinese Hamster Ovary (CHO) cells, human embryonic kidney (HEK) cells, or myeloma cells that do not otherwise produce the protein. Exemplary cells used for expressing a protein are CHO cells, myeloma cells or HEK cells. Molecular cloning techniques to achieve these ends are known in the art and described, for example in Ausubel et al., (editors), Current Protocols in Molecular Biology, Greene Pub. Associates and Wiley-Interscience (1988, including all updates until present) or Sambrook et al., Molecular Cloning: A Laboratory Manual, Cold Spring Harbor Laboratory Press (1989). A wide variety of cloning and in vitro amplification methods are suitable for the construction of recombinant nucleic acids. Methods of producing recombinant antibodies are also known in the art, see, e.g., U.S. Pat. No. 4,816,567 or U.S. Pat. No. 5,530,101.

Following isolation, the nucleic acid is inserted operably linked to a promoter in an expression construct or expression vector for further cloning (amplification of the DNA) or for expression in a cell-free system or in cells.

As used herein, the term “promoter” is to be taken in its broadest context and includes the transcriptional regulatory sequences of a genomic gene, including the TATA box or initiator element, which is required for accurate transcription initiation, with or without additional regulatory elements (e.g., upstream activating sequences, transcription factor binding sites, enhancers and silencers) that alter expression of a nucleic acid, e.g., in response to a developmental and/or external stimulus, or in a tissue specific manner. In the present context, the term “promoter” is also used to describe a recombinant, synthetic or fusion nucleic acid, or derivative which confers, activates or enhances the expression of a nucleic acid to which it is operably linked. Exemplary promoters can contain additional copies of one or more specific regulatory elements to further enhance expression and/or alter the spatial expression and/or temporal expression of said nucleic acid.

As used herein, the term “operably linked to” means positioning a promoter relative to a nucleic acid such that expression of the nucleic acid is controlled by the promoter.

Many vectors for expression in cells are available. The vector components generally include, but are not limited to, one or more of the following: a signal sequence, a sequence encoding a protein (e.g., derived from the information provided herein), an enhancer element, a promoter, and a transcription termination sequence. The skilled artisan will be aware of suitable sequences for expression of a protein. Exemplary signal sequences include prokaryotic secretion signals (e.g., pelB, alkaline phosphatase, penicillinase, Ipp, or heat-stable enterotoxin II), yeast secretion signals (e.g., invertase leader, α factor leader, or acid phosphatase leader) or mammalian secretion signals (e.g., herpes simplex gD signal).

Exemplary promoters active in mammalian cells include cytomegalovirus immediate early promoter (CMV-IE), human elongation factor 1-α promoter (EF1), small nuclear RNA promoters (U1a and U1 b), α-myosin heavy chain promoter, Simian virus 40 promoter (SV40), Rous sarcoma virus promoter (RSV), Adenovirus major late promoter, β-actin promoter; hybrid regulatory element comprising a CMV enhancer/β-actin promoter or an immunoglobulin promoter or active fragment thereof. Examples of useful mammalian host cell lines are monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); or Chinese hamster ovary cells (CHO).

Typical promoters suitable for expression in yeast cells such as for example a yeast cell selected from the group comprising Pichia pastoris, Saccharomyces cerevisiae and S. pombe, include, but are not limited to, the ADH1 promoter, the GAL1 promoter, the GAL4 promoter, the CUP1 promoter, the PHO5 promoter, the not promoter, the RPR1 promoter, or the TEF1 promoter.

Means for introducing the isolated nucleic acid or expression construct comprising same into a cell for expression are known to those skilled in the art. The technique used for a given cell depends on the known successful techniques. Means for introducing recombinant DNA into cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

The host cells used to produce the protein may be cultured in a variety of media, depending on the cell type used. Commercially available media such as Ham's FIO (Sigma), Minimal Essential Medium ((MEM), (Sigma), RPMI-1640 (Sigma), and Dulbecco's Modified Eagle's Medium ((DMEM), Sigma) are suitable for culturing mammalian cells. Media for culturing other cell types discussed herein are known in the art.

### Constant Regions

The present invention encompasses antigen binding proteins and/or antibodies described herein comprising a constant region of an antibody. This includes antigen binding fragments of an antibody fused to an Fc.

Sequences of constant regions useful for producing the proteins of the present invention may be obtained from a number of different sources. In some examples, the constant region or portion thereof of the protein is derived from a human antibody. The constant region or portion thereof may be derived from any antibody class, including IgM, IgG, IgD, IgA and IgE, and any antibody isotype, including IgG1, IgG2, IgG3 and IgG4. In one example, the constant region is human isotype IgG4 or a stabilized IgG4 constant region.

In one example, the Fc region of the constant region has a reduced ability to induce effector function, e.g., compared to a native or wild-type human IgG1 or IgG3 Fc region. In one example, the effector function is antibody-dependent cell-mediated cytotoxicity (ADCC) and/or antibody-dependent cell-mediated phagocytosis (ADCP) and/or complement-dependent cytotoxicity (CDC). Methods for assessing the level of effector function of an Fc region containing protein are known in the art and/or described herein.

In one example, the Fc region is an IgG4 Fc region (i.e., from an IgG4 constant region), e.g., a human IgG4 Fc region. Sequences of suitable IgG4 Fc regions will be apparent to the skilled person and/or available in publically available databases (e.g., available from National Center for Biotechnology Information).

In one example, the constant region is a stabilized IgG4 constant region. The term “stabilized IgG4 constant region” will be understood to mean an IgG4 constant region that has been modified to reduce Fab arm exchange or the propensity to undergo Fab arm exchange or formation of a half-antibody or a propensity to form a half antibody. “Fab arm exchange” refers to a type of protein modification for human IgG4, in which an IgG4 heavy chain and attached light chain (half-molecule) is swapped for a heavy-light chain pair from another IgG4 molecule. Thus, IgG4 molecules may acquire two distinct Fab arms recognizing two distinct antigens (resulting in bispecific molecules). Fab arm exchange occurs naturally in vivo and can be induced in vitro by purified blood cells or reducing agents such as reduced glutathione. A “half antibody” forms when an IgG4 antibody dissociates to form two molecules each containing a single heavy chain and a single light chain.

In one example, a stabilized IgG4 constant region comprises a proline at position 241 of the hinge region according to the system of Kabat (Kabat et al., Sequences of Proteins of Immunological Interest Washington DC United States Department of Health and Human Services, 1987 and/or 1991). This position corresponds to position 228 of the hinge region according to the EU numbering system (Kabat et al., Sequences of Proteins of Immunological Interest Washington DC United States Department of Health and Human Services, 2001 and Edelman et al., Proc. Natl. Acad. USA, 63, 78-85, 1969). In human IgG4, this residue is generally a serine. Following substitution of the serine for proline, the IgG4 hinge region comprises a sequence CPPC. In this regard, the skilled person will be aware that the “hinge region” is a proline-rich portion of an antibody heavy chain constant region that links the Fc and Fab regions that confers mobility on the two Fab arms of an antibody. The hinge region includes cysteine residues which are involved in inter-heavy chain disulfide bonds. It is generally defined as stretching from Glu226 to Pro243 of human IgG1 according to the numbering system of Kabat. Hinge regions of other IgG isotypes may be aligned with the IgG1 sequence by placing the first and last cysteine residues forming inter-heavy chain disulphide (S—S) bonds in the same positions (see for example WO2010/080538).

Additional examples of stabilized IgG4 antibodies are antibodies in which arginine at position 409 in a heavy chain constant region of human IgG4 (according to the EU numbering system) is substituted with lysine, threonine, methionine, or leucine (e.g., as described in WO2006/033386). The Fc region of the constant region may additionally or alternatively comprise a residue selected from the group consisting of: alanine, valine, glycine, isoleucine and leucine at the position corresponding to 405 (according to the EU numbering system). Optionally, the hinge region comprises a proline at position 241 (i.e., a CPPC sequence) (as described above).

In another example, the Fc region is a region modified to have reduced effector function, i.e., a “non-immunostimulatory Fc region”. For example, the Fc region is an IgG1 Fc region comprising a substitution at one or more positions selected from the group consisting of 268, 309, 330 and 331. In another example, the Fc region is an IgG1 Fc region comprising one or more of the following changes E233P, L234V, L235A and deletion of G236 and/or one or more of the following changes A327G, A330S and P331S (Armour et al., Eur J Immunol. 29:2613-2624, 1999; Shields et al., J Biol Chem. 276(9):6591-604, 2001). Additional examples of non-immunostimulatory Fc regions are described, for example, in Dall'Acqua et al., J Immunol. 177: 1129-1138 2006; and/or Hezareh J Virol; 75: 12161-12168, 2001).

In another example, the Fc region is a chimeric Fc region, e.g., comprising at least one C_{H}2 domain from an IgG4 antibody and at least one C_{H}3 domain from an IgG1 antibody, wherein the Fc region comprises a substitution at one or more amino acid positions selected from the group consisting of 240, 262, 264, 266, 297, 299, 307, 309, 323, 399, 409 and 427 (EU numbering) (e.g., as described in WO2010/085682). Exemplary substitutions include 240F, 262L, 264T, 266F, 297Q, 299A, 299K, 307P, 309K, 309M, 309P, 323F, 399S, and 427F.

### Additional Modifications

The present invention also contemplates additional modifications to an antibody or antigen binding protein comprising an Fc region or constant region.

For example, the antibody comprises one or more amino acid substitutions that increase the half-life of the protein. For example, the antibody comprises a Fc region comprising one or more amino acid substitutions that increase the affinity of the Fc region for the neonatal Fc region (FcRn). For example, the Fc region has increased affinity for FcRn at lower pH, e.g., about pH 6.0, to facilitate Fc/FcRn binding in an endosome. In one example, the Fc region has increased affinity for FcRn at about pH 6 compared to its affinity at about pH 7.4, which facilitates the re-release of Fc into blood following cellular recycling. These amino acid substitutions are useful for extending the half-life of a protein, by reducing clearance from the blood.

Exemplary amino acid substitutions include T250Q and/or M428L or T252A, T254S and T266F or M252Y, S254T and T256E or H433K and N434F according to the EU numbering system. Additional or alternative amino acid substitutions are described, for example, in US20070135620 or U.S. Pat. No. 7,083,784.

### Antibody Binding Domain Containing Proteins

### Single-Domain Antibodies

In some examples, an antigen binding protein of the invention is or comprises a single-domain antibody (which is used interchangeably with the term “domain antibody” or “dAb”). A single-domain antibody is a single polypeptide chain comprising all or a portion of the heavy chain variable region of an antibody. In certain examples, a single-domain antibody is a human single-domain antibody (Domantis, Inc., Waltham, MA; see, e.g., U.S. Pat. No. 6,248,516).

### Diabodies, Triabodies, Tetrabodies

In some examples, a protein of the invention is or comprises a diabody, triabody, tetrabody or higher order protein complex such as those described in WO98/044001 and/or WO94/007921.

For example, a diabody is a protein comprising two associated polypeptide chains, each polypeptide chain comprising the structure VL-X-VH or VH-X-VL, wherein VL is an antibody light chain variable region, VH is an antibody heavy chain variable region, X is a linker comprising insufficient residues to permit the VH and VL in a single polypeptide chain to associate (or form an Fv) or is absent, and wherein the VH of one polypeptide chain binds to a VL of the other polypeptide chain to form an antigen binding domain, i.e., to form a Fv molecule capable of specifically binding to one or more antigens. The VL and VH can be the same in each polypeptide chain or the VL and VH can be different in each polypeptide chain so as to form a bispecific diabody (i.e., comprising two Fvs having different specificity).

Single Chain Fv (scFv)

The skilled artisan will be aware that scFvs comprise VH and VL regions in a single polypeptide chain and a polypeptide linker between the VH and VL which enables the scFv to form the desired structure for antigen binding (i.e., for the VH and VL of the single polypeptide chain to associate with one another to form a Fv). For example, the linker comprises in excess of 12 amino acid residues with (Gly₄Ser)₃ being one of the more favored linkers for a scFv.

The present invention also contemplates a disulfide stabilized Fv (or diFv or dsFv), in which a single cysteine residue is introduced into a FR of VH and a FR of VL and the cysteine residues linked by a disulfide bond to yield a stable Fv.

Alternatively, or in addition, the present invention encompasses a dimeric scFv, i.e., a protein comprising two scFv molecules linked by a non-covalent or covalent linkage, e.g., by a leucine zipper domain (e.g., derived from Fos or Jun). Alternatively, two scFvs are linked by a peptide linker of sufficient length to permit both scFvs to form and to bind to an antigen, e.g., as described in US20060263367.

### Heavy Chain Antibodies

Heavy chain antibodies differ structurally from many other forms of antibodies, in so far as they comprise a heavy chain, but do not comprise a light chain. Accordingly, these antibodies are also referred to as “heavy chain only antibodies”. Heavy chain antibodies are found in, for example, camelids and cartilaginous fish (also called IgNAR).

The variable regions present in naturally occurring heavy chain antibodies are generally referred to as “VHH domains” in camelid antibodies and V-NAR in IgNAR, in order to distinguish them from the heavy chain variable regions that are present in conventional 4-chain antibodies (which are referred to as “VH domains”) and from the light chain variable regions that are present in conventional 4-chain antibodies (which are referred to as “VL domains”).

A general description of heavy chain antibodies from camelids and the variable regions thereof and methods for their production and/or isolation and/or use is found inter alia in the following references WO94/04678, WO97/49805 and WO 97/49805.

A general description of heavy chain antibodies from cartilaginous fish and the variable regions thereof and methods for their production and/or isolation and/or use is found inter alia in WO2005/118629.

### Other Antibodies and Proteins Comprising Antigen Binding Domains Thereof

The present invention also contemplates other antibodies and proteins comprising antigen-binding domains thereof, such as:
(i) “key and hole” bispecific proteins as described in U.S. Pat. No. 5,731,168;
(ii) heteroconjugate proteins, e.g., as described in U.S. Pat. No. 4,676,980;
(iii) heteroconjugate proteins produced using a chemical cross-linker, e.g., as described in U.S. Pat. No. 4,676,980; and
(iv) Fabs (e.g., as described in EP19930302894).

### Transforming Growth Factor β Receptor (TGFβR), Extracellular Domain (ECD) or Ligand Binding Fragment Thereof

The present invention provides molecules, optionally in the form of fusion proteins, which comprise an antigen binding protein comprising a tumour antigen binding domain, with a Transforming growth factor β receptor (TGFβR), extracellular domain (ECD) or ligand binding fragment thereof.

In any embodiment of the invention, a Transforming growth factor β receptor (TGFβR), extracellular domain (ECD) or ligand binding fragment thereof, may be derived from TGFβRI, TGFβRII, or TGFβRIII. Typically, the ECD or ligand binding fragment thereof comprises a polypeptide sequence sufficient to bind a TGF-β polypeptide (e.g., TGFβ1, TGFβ2 or TGFβ3).

a. TGF-β Receptor I (TGFβR1)

In an embodiment the ECD or ligand binding fragment thereof may be derived from a TGFβRI (eg isoform 1) and may comprise all or part of the TGFβRI extracellular domain (amino acid residues 34-126). In some cases, the ECD or ligand binding fragment thereof comprises an amino acid sequence having at least 60%, at least 70%, at least 80%, at least 90%, at least 95%, at least 98%, at least 99%, or 100%, aa sequence identity to at least 70, at least 80, at least 90, at least 100, or 103 amino acid residues of the following TGFβRI extracellular domain (ECD) amino acid sequence: LQCFCHLCTKDNFTCVTDGLCFVSVTETTDKVIHNSMCIAEIDLIPRDRPFVCAPSSKTG SVTTTYCCNQDHCNKIELPTTVKSSPGLGPVEL (SEQ ID NO: 246).

b. TGF-β Receptor II (TGFβR11)

In any embodiment the ECD or ligand binding fragment thereof may be derived from a TGFβRII (eg, isoform A) and may comprise all or part of the TGFβRII ECD sequence (amino acid residues 24 to 177). In some cases, a suitable TGFβRII isoform A polypeptide may comprise an amino acid sequence having at least 60%, at least 70%, at least 80%, at least 90%, at least 95%, at least 98%, at least 99%, or 100%, aa sequence identity to at least 70, at least 80, at least 90, at least 100, at least 110, at least 120, at least 130, at least 140, at least 150 or at least 154 amino acid residues of the following TGFβRII isoform A ECD sequence: IPPHVQKSDVEMEAQKDEIICPSCNRTAHPLRHINNDMIVTDNNGAVKFPQLCKFCDVR FSTCDNQKSCMSNCSITSICEKPQEVCVAVWRKNDENITLETVCHDPKLPYHDFILEDA ASPKCIMKEKKKPGETFFMCSCSSDECNDNIIFSEE (SEQ ID NO: 247).

In any embodiment the ECD or ligand binding fragment thereof may be derived from a TGFβRII (e.g., isoform B) and may comprise all or part of the TGFβRII ECD sequence (amino acid residues 24 to 166). In some cases, a suitable TGFβRII isoform B polypeptide comprises an amino acid sequence having at least 60%, at least 70%, at least 80%, at least 90%, at least 95%, at least 98%, at least 99%, or 100%, amino acid sequence identity to at least 70, at least 80, at least 90, at least 100, or 103 amino acid residues of the TGFβRII isoform B ECD sequence: IPPHVQKSVNNDMIVTDNNGAVKFPQLCKFCDVRFSTCDNQKSCMSNCSITSICEKPQ EVCVAVWRKNDENITLETVCHDPKLPYHDFILEDAASPKCIMKEKKKPGETFFMCSCS SDECNDNIIFSEEYNTSNPDLLLVIFQ (SEQ ID NO: 248). Any one or more of F30, D32, S52, E55, or D118 (underlined in the above sequence) may be substituted by an amino acid other than the naturally occurring aa at those positions (e.g. alanine).

In any embodiment, the TGFβRII isoform B polypeptide may comprise the polypeptide of SEQ ID NO: 248 with a D118A and/or D118R substitution. A suitable TGFβRII isoform B polypeptide may also comprise the peptide of SEQ ID NO: 248 with a D118A and/or D118R substitution and one or more of the following substitutions: F30A, D32N, S52L or E55A.

In any embodiment the ECD or ligand binding fragment thereof may be derived from a TGFβRII (e.g., isoform B) and may comprise all or part of the TGFβRII ECD sequence (amino acid residues 24 to 166). In some cases, a suitable TGFβRII isoform B polypeptide comprises an amino acid sequence having at least 60%, at least 70%, at least 80%, at least 90%, at least 95%, at least 98%, at least 99%, or 100%, amino acid sequence identity to at least 70, at least 80, at least 90, at least 100, or 103 amino acid residues of the TGFβRII isoform B ECD sequence: IPPHVQKSVNNDMIVTDNNGAVKFPQLCKFCDVRFSTCDNQKSCMSNCSITSICEKPQ EVCVAVWRKNDENITLETVCHDPKLPYHDFILEDAASPKCIMKEKKKPGETFFMCSCS SDECNDNIIFSEEYNTSNPD (SEQ ID NO: 249) or TIPPHVQKSVNNDMIVTDNNGAVKFPQLCKFCDVRFSTCDNQKSCMSNCSITSICEKP QEVCVAVWRKNDENITLETVCHDPKLPYHDFILEDAASPKCIMKEKKKPGETFFMCSC SSDECNDNIIFSEEYNTSNPD (SEQ ID NO: 321). Any one or more of F30, D32, S52, E55, or D118 (underlined in the above sequences) (numbering per SEQ ID NO: 249) or positions equivalent thereto (eg in SEQ ID NO: 321, these residues are at F31, D33, S53, E56 and D119, respectively), may be substituted by an amino acid other than the naturally occurring aa at those positions (e.g. alanine).

In any embodiment, the TGFβRII isoform B polypeptide may comprise the polypeptide of SEQ ID NO: 249 with a D118A and/or D118R substitution (or SEQ ID NO: 321, with substitutions at equivalent positions). A suitable TGFβRII isoform B polypeptide may also comprise the peptide of SEQ ID NO: 249 with a D118A and/or D118R substitution and one or more of the following substitutions: F30A, D32N, S52L or E55A. A suitable TGFβRII isoform B polypeptide may also comprise the peptide of SEQ ID NO: 321 with a D119A and/or D119R substitution and one or more of the following substitutions: F31A, D33N, S53L or E56A.

In any embodiment, the ECD derived from TGFβRII may comprise a modification such as: a deletion of lengths of 1 to 25 aa within the first 25 amino acids at the N-terminal (e.g. Δ14, Δ25) and/or substitutions at one or more of L27, F30, D32, S49, 150, T51, S52, I53, E55, V77, D118, and/or E119 including any one of substitutions in SEQ ID NO: 248 or s249: L27A, F30A, D32A, D32N, S49A, 150A, T51A, S52A, S52L, 153A, E55A, V77A, D118A, D118R, E119A, and/or E119Q. See e.g. J. Groppe et al. Mol Cell 29, 157-168, (2008) and De Crescenzo et al. JMB 355, 47-62 (2006).

In one aspect, the aspartic acid at position 118 (D118) of the mature TGFβRII B isoform (SEQ ID NO: 248 or 249) may be replaced by an amino acid other than Asp or Glu, such as Ala, giving rise to a D118A substitution, or by Arg, giving rise to a D118R substitution. The Asp residues corresponding to D118 are indicated in SEQ ID NOs. 248 and 249. N-terminal deletions of lengths of 1 to 25 aa (e.g. a Δ25 deletion) and/or substitutions at F24 (e.g. an F24A substitution) may also be combined with D118 substitutions (e.g. D118A or D118R). N-terminal deletions of lengths of 1 to 25 amino acid (e.g. a Δ25 deletion) and/or substitutions at F24 (e.g. an F24A substitution) may also be combined with substitutions at any of L27, F30, D32, S49, 150, T51, S52, I53, E55, V77, D118, and/or E119 (e.g. D118A), particularly any of the specific substitutions recited for those locations in SEQ ID NO: 248 or 249 described above.

Deletions at the N-terminus of the TGFβRII polypeptides may also result in loss of TGFβRI interactions, preventing the ECD or ligand binding fragment thereof that comprises a TGFβRII polypeptide from acting as a constitutively active complex that engages and activates TGFβRI signaling. A 14 amino acid N-terminal deletion (Δ14) of the TGFβRII polypeptide substantively reduces the interaction with TGFβRI, and a Δ25 aa N-terminal deletion appears to completely abrogate the interaction with TGFβRI. N-terminal deletions also substantially alter the pI of the protein, with the Δ14 TGFβRII ECD mutant displaying a pI of about 4.5-5.0 (approximately 4.74). Accordingly, TGF-β constructs or complexes may comprise TGFβRII ECD polypeptides with N-terminal deletions of lengths of 14 to 25 amino acids (e.g. 14, 15, 16, 17, 18, 19, 20, 21, 22, 23, 24, or 25 amino acids). Modified ECD sequences, including those that limit interactions with TGFβRI, and that may be used for the ECD or ligand binding fragment thereof, are described in the paragraphs that follow.

In any embodiment, the ECD or ligand binding fragment thereof comprises a sequence having at least 60%, at least 70%, at least 80%, at least 90%, at least 95%, at least 98%, at least 99%, or 100%, aa sequence identity to at least 70, at least 80, at least 90, at least 100, or 103 amino acid residues of the TGFβRII isoform B ECD sequence: IPPHVQKSVNNDMIVTDNNGAVKFPQLCKFCDVRFSTCDNQKSCMSNCSI TSICEKPQEVCVAVWRKNDENITLETVCHDPKLPYHDFILEDAASPKCIMKEKKKPGET FFMCSCSSDECNDNIIFSEE (SEQ ID NO: 250). Any one or more of F30, D32, S52, E55, or D118 may be substituted by an amino acid other than the naturally occurring aa at those positions (e.g. alanine).

In any embodiment, the ECD or ligand binding fragment thereof comprises the peptide of SEQ ID NO: 250 with a D118A substitution. In another embodiment, the ECD or ligand binding fragment thereof comprises the polypeptide of SEQ ID NO: 250 with a D118A substitution and one or more of a F30A, D32N, S52L and/or E55A substitution.

Combinations of N-terminal deletions of TGFβRII, such as those of lengths of 14 to 25 amino acid residues (e.g., 14, 15, 16, 17, 18, 19, 20, 21, 22, 23, 24, or 25 amino acid residues), that block inadvertent cell signalling due to the bound TGF-β/TGFβRII complex interacting with endogenous TGFβRI, may also be combined with other TGFβRII ECD substitutions, including any one or more of: F30, D32, S52, E55, and/or D118. Combinations of deletions and substitutions limits cell signalling to that which occurs through the cell's endogenous TGFβRI and TGFβRII receptors.

In an embodiment, the ECD or ligand binding fragment thereof comprises a sequence having at least 60%, at least 70%, at least 80%, at least 90%, at least 95%, at least 98%, at least 99%, or 100%, amino acids sequence identity to at least 70, at least 80, at least 90, at least 100, or 103 amino acid residues of the TGFβRII isoform B ECD sequence: TDNNGAVKFPQLCKFCDVRFSTCDNQKSCMSNCSITSICEKPQEVCVAVWRKNDENIT LETVCHDPKLPYHDFILEDAASPKCIMKEKKKPGETFFMCSCSSDECNDNIIFSEE (SEQ ID NO: 251), which has an N-terminal deletion of amino acids 1-14 (Δ14). Any one or more of: F30, D32, S52, E55, or D118 may be substituted by an amino acid other than the naturally occurring aa at those positions (e.g. alanine).

In an embodiment, the ECD or ligand binding fragment thereof comprises the peptide of SEQ ID NO: 250 with a D118A substitution. In another embodiment, the ECD or ligand binding fragment thereof comprises the polypeptide of SEQ ID NO: 251 with a D118A substitution, and is further substituted with one or more of: F30A, D32N, S52L or E55A. In an embodiment, the ECD or ligand binding fragment thereof comprises sequence having at least 60%, at least 70%, at least 80%, at least 90%, at least 95%, at least 98%, at least 99%, or 100%, aa sequence identity to at least 70, at least 80, at least 90, at least 100, or 103 amino acid residues of the sequence: QLCKFCDVRFSTCDNQKSCMSNCSITSICEKPQEVCVAVWRKNDENITLETVCHDPKL PYHDFILEDAASPKCIMKEKKKPGETFFMCSCSSDECNDNIIFSEE (SEQ ID NO: 252), which has residues 1-25 (Δ25) deleted. Any one or more of: F30, D32, S52, E55, or D118 may be substituted by an amino acid other than the naturally occurring aa at those positions (e.g. alanine).

In any embodiment, the ECD or ligand binding fragment thereof comprises the polypeptide of SEQ ID NO: 252 with a D118A substitution. In an embodiment, the ECD or ligand binding fragment thereof comprises the peptide of SEQ ID NO: 252 with a D118A substitution and one or more of the following substitutions: F30A, D32N, S52L or E55A. In an embodiment, the ECD or ligand binding fragment thereof comprises the peptide of SEQ ID NO: 252 with D118A and F30A substitutions. In another embodiment, the ECD or ligand binding fragment thereof comprises the peptide of SEQ ID NO: 252 with D118A and D32N substitutions. In another embodiment, the ECD or ligand binding fragment thereof comprises the peptide of SEQ ID NO: 252 with D118A and S52L substitutions. In an embodiment, the ECD or ligand binding fragment thereof comprises the peptide of SEQ ID NO: 252 with D118A and E55A substitutions.

c. TGF-β Receptor III (TGFβRIII)

In an embodiment, the ECD or ligand binding fragment thereof may be derived from a TGFβRIII (e.g. isoform A and isoform B), and may comprise all or part of a TGFβRIII ECD (amino acids 27-787 of the A isoform or 27-786 of the B isoform). In some cases, the ECD or ligand binding fragment thereof comprises an amino acid sequence having at least 60%, at least 70%, at least 80%, at least 90%, at least 95%, at least 98%, at least 99%, or 100%, aa sequence identity to at least 70, at least 80, at least 90, at least 100, or 120 amino acids of TGFβRIII A isoform or B isoform ECD sequences (ie SEQ ID NOs: 253 or 254).

### Linkers and Other Means for Joining Antigen Binding Proteins and the Extracellular Domain (ECD) or Ligand Binding Fragment of a TGFβR.

In any embodiment of the invention, the extracellular domain (ECD) or ligand binding fragment of a transforming growth factor β receptor (TGFβR) is joined directly to the tumour antigen binding protein, or is joined via a linker.

Typically, the tumour antigen binding protein and the extracellular domain (ECD) or ligand binding fragment of a transforming growth factor β receptor (TGFβR) are linked via the C-terminus of the heavy chain of the antigen binding protein. However, it will be appreciated the ECD or ligand binding fragment of a TGFβR may be joined via the C-terminus of the light chain of the antigen binding protein, or via any non-antigen binding region of the tumour antigen binding protein. For example in instances wherein the tumour antigen binding protein is an antibody or Fab, the ECD or ligand binding fragment of a TGFβR may be joined to any amino acid of any constant region of the protein or any region of a variable domain which is not directly involved in antigen binding.

It will be appreciated that any number of different linkers (or “spacer”) may be used, including peptide linkers, carbohydrate linkers (such as PEG-based linkers) or via a chemical linker.

Linker” herein is also referred to as “linker sequence”, “spacer”, “tethering sequence” or grammatical equivalents thereof. Homo- or hetero-bifunctional linkers as are well known (see, 1994 Pierce Chemical Company catalog, technical section on cross-linkers, pages 155-200, incorporated entirely by reference). A number of strategies may be used to covalently link molecules together. These include, but are not limited to polypeptide linkages between N- and C-termini of proteins or protein domains, linkage via disulfide bonds, and linkage via chemical cross-linking reagents. In one aspect of this embodiment, the linker is a peptide bond, generated by recombinant techniques or peptide synthesis. The linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used. In certain embodiments of the present invention the linker may have a length of 1, 2, 3, 4, 5, 6, 7, 8, 9, 10, 11, 12, 13, 14, 15, 16, 17, 18, 19, 20, 21 or 22 or more amino acids.

Useful linkers include glycine-serine polymers, including for example (GS)n, (GSGGS)n, (GGGGS)n, and (GGGS)n, where n is an integer of at least one, glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

The linker may be a flexible linker (such as those comprising repeats of glycine and serine residues), a rigid linker (such as those comprising glutamic acid and lysine residues, flanking alanine repeats) and/or a cleavable linker (such as sequences that are susceptible by protease cleavage). Examples of such linkers are known to the skilled person and are described for example, in Chen et al., (2013) Advanced Drug Delivery Reviews, 65: 1357-1369 and in Holliger, P., et al. (1993) Proc. Natl. Acad. Sci. USA 90:6444-6448; Poljak, R. J., et al. (1994) Structure 2:1121-1123; both of which are incorporated herein by reference.

In some aspects, the linker may include the amino acids glycine and serine in various lengths and combinations. In some aspects, the peptide linker can include the sequence Gly-Gly-Ser (GGS), Gly-Gly-Gly-Ser (GGGS) or Gly-Gly-Gly-Gly-Ser (GGGGS) and variations or repeats thereof. In some aspects, the peptide linker can include the amino acid sequence GGGGS (a linker of 6 amino acids in length) or even longer. The linker may a series of repeating glycine and serine residues (GS) of different lengths, i.e., (GS)ₙ where n is any number from 1 to 15 or more. For example, the linker may be (GS)₃ (i.e., GSGSGS) or longer (GS)₁₁ or longer. It will be appreciated that a can be any number including 1, 2, 3, 4, 5, 6, 7, 8, 9, 10, 11 or more. Fusion proteins having linkers of such length are included within the scope of the present invention. Similarly, the linker may be a series of repeating glycine residues separated by serine residues. For example (GGGGS)₃ (i.e., the linker may comprise the amino acid sequence GGGGSGGGGSGGGGS (G4S)₃) and variations thereof.

The peptide linker may consist of a series of repeats of Thr-Pro (TP) comprising one or more additional amino acids N and C terminal to the repeat sequence. For example, the linker may comprise or consist of the sequence GTPTPTPTPTGE.

In further aspects, the linker may be a short and/or alpha-helical rigid linker (e.g. A(EAAAK)3A, PAPAP or a dipeptide such as LE).

In certain aspects, the linker may be flexible and cleavable. Such linkers preferably comprise one or more recognition sites for a protease to enable cleavage.

In certain aspect, the linker may be derived from an antibody hinge region. Hinge regions sequences from any antibody isotype may be used, including for example hinge sequences from IgG1, IgG2, IgG3, and/or IgG4. Linker sequences may also include any sequence of any length of CL/CH1 domain but not all residues of CL/CH1 domain; for example the first 5-12 amino acid residues of the CL/CH1 domains. Linkers can be derived from immunoglobulin heavy chains of any isotype, including for example Cγ1, Cγ2, Cγ3, Cγ4, Cα1, Cα2, Cδ, Cε, and Cμ. Linkers can be derived from immunoglobulin light chain, for example Cκ or Cλ. Linker sequences may also be derived from other proteins such as Ig-like proteins (e.g. TCR, FcR, KIR), hinge region-derived sequences, and other natural sequences from other proteins.

In still further embodiments, the antigen binding protein and extracellular domain (ECD) or ligand binding fragment of a TGFβR may be provided as separate proteins which can become joined in vivo. For example, the provision of complementary binding sequences in one moiety and a binding sequence in the other moiety are contemplated within the scope of the invention to facilitate protein-protein interaction between the two moieties. In one example, the invention contemplates the use of leucine zipper (LZ) protein-protein interaction domains in both moieties, such that the moieties join in vivo to form a fusion comprising a first moiety comprising the tumour antigen binding domain, and a second moiety comprising the extracellular domain (ECD) or ligand binding fragment of a TGFβR. The design and use of LZ zippers are well known to the skilled person in the art.

Other approaches for facilitating joining of the antigen binding protein and extracellular domain (ECD) or ligand binding fragment of a TGFβR may include the use of cysteine residues to facilitate formation of disulphide bonding between the two moieties.

### Isolation of Proteins

Methods for isolating a protein are known in the art and/or described herein.

Where an antigen binding protein is secreted into culture medium, supernatants from such expression systems can be first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants. Alternatively, or additionally, supernatants can be filtered and/or separated from cells expressing the protein, e.g., using continuous centrifugation.

The antigen binding protein or fusion proteins of the invention, prepared from the cells can be purified using, for example, ion exchange, hydroxyapatite chromatography, hydrophobic interaction chromatography, gel electrophoresis, dialysis, affinity chromatography (e.g., protein A affinity chromatography or protein G chromatography), or any combination of the foregoing. These methods are known in the art and described, for example in WO99/57134 or Ed Harlow and David Lane (editors) Antibodies: A Laboratory Manual, Cold Spring Harbor Laboratory, (1988).

The skilled artisan will also be aware that a protein can be modified to include a tag to facilitate purification or detection, e.g., a poly-histidine tag, e.g., a hexa-histidine tag, or an influenza virus hemagglutinin (HA) tag, or a Simian Virus 5 (V5) tag, or a FLAG tag, or a glutathione S-transferase (GST) tag. The resulting protein is then purified using methods known in the art, such as, affinity purification. For example, a protein comprising a hexa-his tag is purified by contacting a sample comprising the protein with nickel-nitrilotriacetic acid (Ni-NTA) that specifically binds a hexa-his tag immobilized on a solid or semi-solid support, washing the sample to remove unbound protein, and subsequently eluting the bound protein. Alternatively, or in addition a ligand or antibody that binds to a tag is used in an affinity purification method.

### Linking of Radioisotopes to Proteins

In any embodiment of the invention, the molecules or fusion proteins herein described may be directly or indirectly linked to a therapeutic agent. In any embodiment, the bioconjugates herein described are capable of being indirectly linked to a therapeutic agent via a chelator moiety or linker group. Preferably, the therapeutic agent is a radioisotope. In preferred embodiments, the therapeutic agent is an alpha emitting radioisotope or a beta emitting radioisotope.

Examples of suitable isotopes include: actinium-225 (225Ac), astatine-211 (211At), bismuth-212 and bismuth-213 (²¹²Bi, ²¹³Bi), copper-67 (⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²⁴Ra), samarium-153 (¹⁵³Sm), scandium-47 (⁴⁷Sc), strontium-90 (⁹⁰Sr), and yttrium-90 (⁹⁰Y. In some embodiments, the radionuclide conjugated with the molecule is lutetium-¹⁷⁷.

It will be understood that the radioisotopes may be conjugated to the molecules or fusion proteins of the invention directly (via a chelating agent or prosthetic group or linker) or indirectly via binding to single or multiple amino acid residues in the antibody (e.g. halogenation of tyrosine residues).

In alternative embodiments, chelating agents or linkers may be used in order to conjugate the radioisotope to the antibody. In one example, the antibodies can be conjugated to a chelating moiety, selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1, 4,7,10-tetraazacyclododecane-NN′,N″(N′″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8), 11-tetraacetic acid, Te2A (4,11-bis(carboxymethyl)-1,4,8,11-tetraazabicyclo[6.6.2]hexadecane), HBED, DFO (Desferrioxamine), DFOsq (DFO-squaramide) and HOPO (3,4,3-(LI-1,2-HOPO) or other chelating agent as described herein.

Chelators with radiometals and other halogenated radioisotopes may be bound to the molecules of the invention, or antibodies, antibody fragments thereof herein described, via one or more amino acid residues or reactive moieties in the antibody, including but not limited to one or more lysine residues, tyrosine residues or thiol moieties.

In another example, the antibody, antibody fragment thereof or molecule is conjugated to a bifunctional linker, for example, bromoacetyl, thiols, succinimide ester, TFP ester, a maleimide, or using any amine or thiol-modifying chemistry known in the art.

The skilled person will be familiar with standard methods for conjugating chelating agents to antibodies and derivatives or fragments thereof. In addition, the skilled person will be familiar with approaches for selecting a relevant chelating agent for pairing with a radiometal, for example as described in Chem. Soc. Rev., 2014,43, 260, incorporated herein by reference.

### Assaying Activity of an Antigen Binding Protein

It will be within the purview of the person skilled in the art to identify a relevant antigen binding protein for use in binding to a cancer antigen, in order to make use of the molecules of the invention. Examples of various cancer antigens are further described herein and are further known in the art. The skilled person will be able to determine which antigens are preferentially expressed by the cancer to be treated.

Having identified the antigen (or antigens) characterising the cancer to be treated, the skilled person will then be able to ascertain various antigen binding proteins for binding to said antigens, and to then be able to generate a molecule of the invention, comprising said antigen binding protein. Examples of known antigen binding proteins for binding to known antigens associated with cancer are further described herein. It is well within the skill set of the skilled person to be able to join any ECD of a TGFβR, including one as described herein, to any desired antigen binding protein, by following the information provided herein and utilising general skills in the art. Further the skilled person will be able to confirm the ability of the molecule to bind to both the cancer antigen and to TGFβ.

Methods for assessing binding to a protein are known in the art, e.g., as described in Scopes (In: Protein purification: principles and practice, Third Edition, Springer Verlag, 1994). Such a method generally involves immobilizing the antigen binding protein and contacting it with labeled antigen. Following washing to remove non-specific bound protein, the amount of label and, as a consequence, bound antigen is detected. Of course, the antigen binding protein can be labeled and the antigen immobilized. Panning-type assays can also be used. Alternatively, or additionally, surface plasmon resonance assays can be used.

### Therapeutic Methods

The molecules of the present invention are useful for treating a number of conditions in which TGFβ inhibition is required in a specific tissue type or cell type. Typically, such conditions include cancer.

In any embodiment, the tumour antigen binding protein may be for binding to an antigen expressed by any cancer, optionally wherein the tumour or cancer is selected from: cystic and solid tumours, bone and soft tissue tumours, including tumours in anal tissue, bile duct, bladder, blood cells, bowel, brain, breast, carcinoid, cervix, eye, oesophagus, head and neck, kidney, larynx, leukaemia, liver, lung, lymph nodes, lymphoma, melanoma, mesothelioma, myeloma, ovary, pancreas, penis, prostate, skin (e.g. squamous cell carcinoma), sarcomas, stomach, testes, thyroid, vagina, vulva. Soft tissue tumours include Benign schwannoma Monosomy, Desmoid tumour, lipo-blastoma, lipoma, uterine leiomyoma, clear cell sarcoma, dermatofibrosarcoma, Ewing sarcoma, extraskeletal myxoid chondrosarcoma, liposarcooma myxoid, Alveolar rhabdomyosarcoma and synovial sarcoma. Specific bone tumours include nonossifying fibroma, unicameral bone cyst, enchon-droma, aneurismal bone cyst, osteoblastoma, chondroblastoma, chondromyxofibroma, ossifying fibroma and adamantinoma, Giant cell tumour, fibrous dysplasia, Ewing's sarcoma eosinophilic granuloma, osteosarcoma, chondroma, chondrosarcoma, malignant fibrous histiocytoma and metastatic carcinoma. Leukaemias include acute lymphoblastic, acute myeloblastic, chronic lymphocytic and chronic myeloid.

Other examples include breast tumours, colorectal tumours, adenocarcinomas, mesothelioma, bladder tumours, prostate tumours, germ cell tumour, hepatoma/cholongio, carcinoma, neuroendocrine tumours, pituitary neoplasm, small round cell tumour, squamous cell cancer, melanoma, atypical fibroxanthoma, seminomas, nonseminomas, stromal leydig cell tumours, Sertoli cell tumours, skin tumuors, kidney tumours, testicular tumours, brain tumours, ovarian tumours, stomach tumours, oral tumors, bladder tumours, bone tumours, cervical tumors, esophageal tumuors, laryngeal tumours, liver tumours, lung tumours, vaginal tumours and Wilm's tumour.

In some embodiments, the cancer is metastatic cancer. The primary source for the metastatic cancer may be any cancer type known in the art, including those described herein.

Preferably, the cancer is a solid tumour.

Non-limiting examples of tumour antigens are TSA or TAA antigens which include the following: 17-IA-antigen, alpha-fetoprotein (AFP), alpha-actinin-4, A3, antigen specific for A33 antibody, ART-4, B7, Ba 733, BAGE, bcl-2, bcl-6, BCMA, BrE3-antigen, CA125, CAMEL, CAP-1, carbonic anhydrase IX (CAIX), CASP-8/m, CCL19, CCL21, CD1, CDla, CD2, CD3, CD4, CD5, CD8, CD11A, CD14, CD15, CD16, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66α-e, CD67, CD70, CD70L, CD74, CD79a, CD79b, CD80, CD83, CD95, CD123, CD126, CD132, CD133, CD 138, CD 147, CD 154, CD171, CDC27, CDK-4/m, CDKN2A, CEA, CEACAM5, CEACAM6, complement factors (such as C3, C3a, C3b, C5a and C5), colon-specific antigen-p (CSAp), c-Met, CTLA-4, CXCR4, CXCR7, CXCL12, DAM, Dickkopf-related protein (DKK), ED-B fibronectin, EGFR, EGFRvIII, EGP-1 (TROP-2), EGP-2, ELF2-M, Ep-CAM, EphA2, EphA3, fibroblast activation protein (FAP), fibroblast growth factor (FGF), Flt-1, Flt-3, folate binding protein, folate receptor, G250 antigen, gangliosides (such as GC2, GD3 and GM2), GAGE, GD2, gplOO, GPC3, GRO-13, HLA-DR, HM1.24, human chorionic gonadotropin (HCG) and its subunits, HER2, HER3, HMGB-1, hypoxia inducible factor (HIF-1), HIF-la, HSP70-2M, HST-2, la, IFN-gamma, IFN-alpha, IFN-beta, IFN-X, IL-4R, IL-6R, IL-13R, IL13Ralpha2, IL-15R, IL-17R, IL-18R, IL-2, IL-6, IL-8, IL-12, IL-15, IL-17, IL-18, IL-23, IL-25, ILGF, ILGF-1R, insulin-like growth factor-1 (IGF-1), IGF-1R, integrin avP3, integrin α5β1, KC4-antigen, killer-cell immunoglobulin-like receptor (KIR), Kras, KS-1-antigen, KS1-4, La/SSB, LDR/FUT, Le1, macrophage migration inhibitory factor (MIF), MAGE, MAGE-3, MART-1, MART-2, mCRP, MCP-1, melanoma glycoprotein, mesothelin, MIP-1 A, MIP-1B, MIF, mucins (such as MUC1, MUC2, MUC3, MUC4, MUC5ac, MUC13, MUC16, MUM-1/2 and MUM-3), NCA66, NCA95, NCA90, NY-ESO-1, PAM4 antigen, pancreatic cancer mucin, PD-1, PD-L1, PD-1 receptor, placental growth factor, platelet-derived growth factor receptor alpha (PDGFRα), p53, PLAGL2, prostatic acid phosphatase, PSA, PRAME, PSMA, P1GF, RS5, RANTES, SAGE, 5100, survivin, survivin-2B, T101, TAC, TAG-72, tenascin, Thomson-Friedenreich antigens, Tn antigen, TNF-alpha, tumour necrosis antigens, TRAG-3, TRAIL receptors, VEGF, VEGFR and WT-1. It will therefore be appreciated that in any embodiment, the tumour antigen binding protein may be one that is capable of specifically binding to any such antigen.

In preferred embodiments of the invention, the TAA is selected from carbonic anhydrase IX (CAIX) prostate specific membrane antigen (PSMA), PDGFRalpha. La/SSB.

In certain embodiments of the invention, the tumour antigen is not EGFR or EGFRvIII.

In certain embodiment of the invention, the tumour antigen is not PD-1, PD-L1 or PD-L1 receptor.

Optionally, the tumour antigen binding protein may be one selected from, or may comprise an antigen binding domain derived from any one of the following: LL1 (anti-CD74), LL2 or RFB4 (anti-CD22), veltuzumab (hA20, anti-CD20), rituxumab (anti-CD20), obinutuzumab (GA101, anti-CD20), daratumumab (anti-CD38), lambrolizumab (anti-PD-1 receptor), nivolumab (anti-PD-1 receptor), ipilimumab (anti-CTLA-4), RS7 (anti-TROP-2), PAM4 or KC4 (both anti-mucin), MN-14 (anti-CEA), MN-15 or MN-3 (anti-CEACAM6), Mu-9 (anti-colon-specific antigen-p), Immu 31 (an anti-alpha-fetoprotein), RI (anti-IGF-IR), A19 (anti-CD19), TAG-72 (e.g., CC49), Tn, J591 or HuJ591 (anti-PSMA), AB-PG1-XG1-026 (anti-PSMA dimer), D2/B (anti-PSMA), G250 (anti-carbonic anhydrase IX), L243 (anti-HLA-DR) alemtuzumab (anti-CD52), bevacizumab (anti-VEGF), cetuximab (anti-EGFR), gemtuzumab (anti-CD33), ibritumomab tiuxetan (anti-CD20); panitumumab (anti-EGFR); tositumomab (anti-CD20); PAM4 (aka clivatuzumab, anti-mucin), trastuzumab (anti-HER2), pertuzumab (anti-HER2), polatuzumab (anti-CD79b) and anetumab (anti-mesothelin).

In further embodiments, the tumour antigen binding protein may be one selected from, or may comprise an antigen binding domain derived from any one of the following: APOMAB (DAB4), atezolizumab, avelumab, bevacizumab, cemiplimab, cetuximab, dataumumab, dinutuximab, durvalumab, elotuzumab, girentuximab, ipilimumab, isatuximab, J591 or huJ591, mogamulizumab, nectimumumab, nivolumab, obinutuzumab, ofatumumab, olaratumab, panitumumab, pembrolizumab, pertizimab, ramucirumab, rituximab, trastuzumab.

Preferably, the molecules of the present invention are useful for treating cancer that are characterised by the presence of PSMA, CAIX, PDGFRα or La/SSB. For example, the molecules that bind to PSMA are useful for treating cancers characterised by increased expression of PSMA, including prostate cancer, bladder cancer, testicular-embryonal cancer, neuroendocrine cancer, renal cell carcinoma, and breast cancer. The molecules that bind to CAIX may be useful for treating cancers characterised by increased expression of CAIX, including renal cell carcinoma (including clear cell renal cell carcinoma), colon cancer, breast cancer, lung cancer, cervical cancer and melanoma. The molecules that bind to PDGFRα may be useful for treating cancers characterised by increased expression of PDGFRα, including gastrointestinal stromal tumours (GISTs) and other soft tissue sarcomas. The molecules that bind to La/SSB may be useful for treating cancers characterised by increased expression of La/SSB, including cancer cells that have been treated with chemotherapy and/or radiation.

In preferred embodiments, the methods of the present invention comprise:
identifying a subject having cancer who has received, or who is receiving or will receive a treatment for cancer, wherein the treatment is suspected or known to cause an increase in TGFβ activity in the tumour microenvironment, or wherein the tumour microenvironment of the cancer has a high baseline level of TGFβ activity;
administering to the subject, a molecule comprising an antigen binding protein that binds to or specifically binds to an antigen of the cancer (optionally wherein the antigen is CAIX, PSMA, PDGFRα or La/SSB), wherein the molecule further comprises an ECD or ligand binding fragment of a TGFβR as described herein, thereby inhibiting TGFβ activity in the cancer.

In further embodiments, the methods of the present invention comprise:
identifying a subject having cancer; characterised by the expression of carbonic anhydrase IX (CAIX);
administering to the subject, a first treatment for the cancer, wherein the treatment is suspected or known to cause an increase in TGFβ activity in the tumour microenvironment;
administering to the subject, a molecule comprising an antigen binding protein that binds to or specifically binds to carbonic anhydrase IX (CAIX), wherein the molecule further comprises an ECD or ligand binding fragment of a TGFβR as described herein, thereby inhibiting TGFβ activity in the cancer.

In further embodiments, the methods of the present invention comprise:
identifying a subject having cancer, preferably characterised by the expression of prostate specific membrane antigen (PSMA);
administering to the subject, a first treatment for the cancer, wherein the treatment is suspected or known to cause an increase in TGFβ activity in the tumour microenvironment;
administering to the subject, a molecule comprising an antigen binding protein that binds to or specifically binds to PSMA, wherein the molecule further comprises an ECD or ligand binding fragment of a TGFβR as described herein, thereby inhibiting TGFβ activity in the cancer.

In further embodiments, the methods of the present invention comprise:
identifying a subject having cancer; characterised by the expression of PDGFRα;
administering to the subject, a first treatment for the cancer, wherein the treatment is suspected or known to cause an increase in TGFβ activity in the tumour microenvironment;
administering to the subject, a molecule comprising an antigen binding protein that binds to or specifically binds to PDGFRα, wherein the molecule further comprises an ECD or ligand binding fragment of a TGFβR as described herein, thereby inhibiting TGFβ activity in the cancer.

In further embodiments, the methods of the present invention comprise:
identifying a subject having cancer; characterised by the expression of La/SSB;
administering to the subject, a first treatment for the cancer, wherein the treatment is suspected or known to cause an increase in TGFβ activity in the tumour microenvironment;
administering to the subject, a molecule comprising an antigen binding protein that binds to or specifically binds to La/SSB, wherein the molecule further comprises an ECD or ligand binding fragment of a TGFβR as described herein, thereby inhibiting TGFβ activity in the cancer.

Optionally, the treatment for the cancer that is suspected of causing, or causes, an increase in TGFβ activity in the tumour microenvironment may be selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

Optionally, a molecule or composition that is administered may comprise a radionuclide.

In any embodiment, the methods of the present invention may inhibit or prevent cancer treatment-related fibrosis in a subject; reduce or inhibit radiation-induced TGFβ activity in a subject receiving treatment for cancer, and/or may enhance or increase the likelihood of success of treatment with a subsequent cancer treatment, such as an immune checkpoint inhibitor.

It will be understood that the molecule or composition of the invention may be administered as a first-line treatment for the cancer. In such embodiments, the molecule or composition of the invention may comprise an antigen binding protein for binding to the cancer antigen (such as CAIX, PSMA, PDGFRα or La/SSB), joined to an ECD or ligand binding fragment of a TGFβR (preferably an ECD of TGFβR II). In further embodiments, the molecule or composition of the invention may comprise an antigen binding protein for binding to the cancer antigen (such as CAIX, PSMA, PDGFRα or La/SSB), joined to an ECD or ligand binding fragment of a TGFβR (preferably an ECD of TGFβR II) wherein the molecule is further conjugated to a radionuclide for enabling molecular targeted radionuclide therapy at the tumour site. Alternatively or in addition, the molecule or composition may be administered concomitantly with external beam radiation directed to the site of the tumour.

In alternative embodiments, and as should be clear from the above, the molecule or composition of the invention may be administered following an initial treatment of the cancer. In preferred embodiments, the initial treatment of the cancer is one that elicits an increased level of TGFβ in the tumour microenvironment (such as EBR or an alternative molecular targeted treatment). In such examples, the molecule or composition that is administered to the subject may not include a radionuclide. In alternative examples, the molecule that is administered is conjugated to a radionuclide for enabling molecular targeted radionuclide therapy at the tumour site. In instances where the prior treatment is with MTR, the methods of the present invention may comprise subsequent MTR with a molecule of the invention, wherein the prior MTR and subsequent MTR comprise antigen binding proteins for binding to the same tumour antigen. Alternatively, the prior MTR and subsequent MTR comprise antigen binding proteins for binding to different tumour antigens on the same tumour type. Further still, the prior MTR and subsequent MTR comprise antigen binding proteins for binding to the same tumour antigen but wherein the antigen binding proteins bind to different epitopes on the antigen, or wherein the antigen binding proteins bind to the same epitope on the antigen, but comprise differing amino acid sequences.

Administration of two or more therapies according to any method of the present invention may include simultaneous, separate or sequential administration of the two or more different therapies. Accordingly, in any embodiment of the methods of the invention, the administration of two or more molecules, bioconjugates, compositions, antibodies, antibody fragments thereof and other cancer treatments may be provided in the same or different dosage form, and may be administered simultaneously, or separately or sequentially in any order.

In further embodiments, the invention provides a method of reducing or inhibiting radiation-induced TGFβ activity in a subject comprising:
identifying a subject having cancer characterised by the expression of carbonic anhydrase IX (CAIX);
administering to the subject, a molecule comprising an antigen binding protein that binds to or specifically binds to CAIX, wherein the molecule further comprises an ECD or ligand binding fragment of a TGFβR as described herein, thereby inhibiting TGFβ activity in the cancer; optionally wherein the molecule does not comprise a radionuclide; and
administering to the subject an antibody or antibody fragment thereof that binds or specifically binds to CAIX, optionally wherein the antibody or antibody fragment thereof is conjugated to a radionuclide, thereby reducing or inhibiting radiation-induced TGFβ activity in the subject.

In further embodiments, the invention provides a method of reducing or inhibiting radiation-induced TGFβ activity in a subject comprising:
identifying a subject having cancer characterised by the expression of prostate specific membrane antigen (PSMA);
administering to the subject, a molecule comprising an antigen binding protein that binds to or specifically binds to PSMA, wherein the molecule further comprises an ECD or ligand binding fragment of a TGFβR as described herein, thereby inhibiting TGFβ activity in the cancer; optionally wherein the molecule does not comprise a radionuclide; and
administering to the subject an antibody or antibody fragment thereof that binds or specifically binds to PSMA, optionally wherein the antibody or antibody fragment thereof is conjugated to a radionuclide, thereby reducing or inhibiting radiation-induced TGFβ activity in the subject.

In further embodiments, the invention provides a method of reducing or inhibiting radiation-induced TGFβ activity in a subject comprising:
identifying a subject having cancer characterised by the expression of PDGFRα;
administering to the subject, a molecule comprising an antigen binding protein that binds to or specifically binds to PDGFRα, wherein the molecule further comprises an ECD or ligand binding fragment of a TGFβR as described herein, thereby inhibiting TGFβ activity in the cancer; optionally wherein the molecule does not comprise a radionuclide; and
administering to the subject an antibody or antibody fragment thereof that binds or specifically binds to PDGFRα, optionally wherein the antibody or antibody fragment thereof is conjugated to a radionuclide, thereby reducing or inhibiting radiation-induced TGFβ activity in the subject.

In further embodiments, the invention provides a method of reducing or inhibiting radiation-induced TGFβ activity in a subject comprising:
identifying a subject having cancer characterised by the expression of La/SSB;
administering to the subject, a molecule comprising an antigen binding protein that binds to or specifically binds to La/SSB, wherein the molecule further comprises an ECD or ligand binding fragment of a TGFβR as described herein, thereby inhibiting TGFβ activity in the cancer; optionally wherein the molecule does not comprise a radionuclide; and
administering to the subject an antibody or antibody fragment thereof that binds or specifically binds to La/SSB, optionally wherein the antibody or antibody fragment thereof is conjugated to a radionuclide, thereby reducing or inhibiting radiation-induced TGFβ activity in the subject.

In further embodiments, the methods of the present invention comprise:
identifying a subject having cancer characterised by the expression of carbonic anhydrase IX (CAIX);
administering to the subject, a molecule comprising an antigen binding protein that binds to or specifically binds to CAIX, wherein the molecule further comprises an ECD or ligand binding fragment of a TGFβR as described herein, thereby inhibiting TGFβ activity in the cancer; optionally wherein the molecule is conjugated to a radionuclide;
administering to the subject an immune checkpoint inhibitor therapy (CPI therapy).

In further embodiments, the methods of the present invention comprise:
identifying a subject having cancer characterised by the expression of prostate specific membrane antigen (PSMA);
administering to the subject, a molecule comprising an antigen binding protein that binds to or specifically binds to PSMA, wherein the molecule further comprises an ECD or ligand binding fragment of a TGFβR as described herein, thereby inhibiting TGFβ activity in the cancer; optionally wherein the molecule is conjugated to a radionuclide;
administering to the subject an immune checkpoint inhibitor therapy (CPI therapy).

In further embodiments, the methods of the present invention comprise:
identifying a subject having cancer characterised by the expression of PDGFRα;
administering to the subject, a molecule comprising an antigen binding protein that binds to or specifically binds to PDGFRα, wherein the molecule further comprises an ECD or ligand binding fragment of a TGFβR as described herein, thereby inhibiting TGFβ activity in the cancer; optionally wherein the molecule is conjugated to a radionuclide;
administering to the subject an immune checkpoint inhibitor therapy (CPI therapy).

In further embodiments, the methods of the present invention comprise:
identifying a subject having cancer characterised by the expression of La/SSB;
administering to the subject, a molecule comprising an antigen binding protein that binds to or specifically binds to La/SSB, wherein the molecule further comprises an ECD or ligand binding fragment of a TGFβR as described herein, thereby inhibiting TGFβ activity in the cancer; optionally wherein the molecule is conjugated to a radionuclide;
administering to the subject an immune checkpoint inhibitor therapy (CPI therapy).

Optionally the amount of CPI administered is lower than the amount administered for successful monotherapy with the CPI as further described herein.

In any embodiment, the administration of the molecule of the invention and the immune checkpoint inhibitor is separate, consecutive or sequential.

Examples of immune checkpoints and antibody inhibitors that target those checkpoints include anti-CTLA-4 (e.g., Ipilimumab, Tremelimumab, KAHR-102), anti-TIM3 (e.g., F38-2E2. ENUM005), anti-LAG3 (e.g., BMS-986016, IMP701. IMP321, C9B7W), anti-KIR (e.g., Lirilumab, IPH2101, IPH4102), anti-PD-1 (e.g., Nivolumab, Pidilizumab, Pembrolizumab, BMS-936559, atezolizumab, Lambrolizumab, MK-3475. AMP-224, AMP-514, STI-A1110, TSR-042), anti-PD-L1 (e.g., KY-1003 (EP20120194977), MCLA-145, atezolizumab. BMS-936559, MEDI-4736, MSB0010718C, AUR-012, STI-A1010. PCT/US2001/020964, MPDL3280A, AMP-224, Dapirolizumab pegol (CDP-7657), MEDI-4920), anti-CD73 (e.g., AR-42 (OSU-HDAC42, HDAC-42, AR42, AR 42, OSU-HDAC 42, OSU-HDAC-42, NSC D736012, HDAC-42, HDAC 42, HDAC42, NSCD736012, NSC-D736012), MEDI-9447), anti-B7-H3 (e.g., MGA271, DS-5573a, 8H9), anti-CD47 (e.g., CC-90002, TTI-621, VLST-007), anti-BTLA, anti-VISTA, anti-A2aR, anti-B7-1, anti-B7-H4, anti-CD52 (such as alemtuzumab), anti-IL-10, anti-IL-35, anti-CSF1R (e.g., FPA008), anti-NKG2A (e.g., monalizumab), anti-MICA (e.g., IPH43), and anti-CD39.

Examples of suitable PD-1 inhibitors that may be used in accordance with the invention include Keytruda (pembrolizumab), Opdivo (nivolumab), AGEN 2034, BGB-A317, BI-754091, CBT-501 (genolimzumab), MED10680, MGA012, PDR001, PF-06801591, REGN2810 (SAR439684), and TSR-042 or those that are disclosed in U.S. Pat. No. 8,008,449. Other anti-PD-1 mAbs have been described in, for example, U.S. Pat. Nos. 6,808,710, 7,488,802, 8,168,757 and 8,354,509, and PCT Publication No. WO 2012/145493.

Nivolumab (also known as “Opdivo®”; formerly designated 5C4, BMS-936558, MDX-1106, or ONO4538) is a fully human IgG4 (S228P) PD-1 immune check point inhibitor Ab that selectively prevents interaction with PD-1 ligands (PD-L1 and PD-L2), thereby blocking the down-regulation of antitumor T-cell functions (U.S. Pat. No. 8,008,449).

Pembrolizumab (also known as “Keytruda®”, lambrolizumab, and MK-3475) is a humanized monoclonal IgG4 antibody directed against human cell surface receptor PD-1 (programmed death-1 or programmed cell death-1). Pembrolizumab is described for example, in U.S. Pat. Nos. 8,354,509 and 8,900,587). Pembrolizumab has been approved by the FDA for the treatment of relapsed or refractory melanoma.

Other suitable PD-1 inhibitors include Libtayo (cemiplimab), Blincyto (blinatumomab), Dostarlimab, Spartalizumab, Cetrelimab, Pidilizumab and BI-754091.

Anti-PD-1 Abs suitable for use in the disclosed methods or compositions are Abs that bind to PD-1 with high specificity and affinity, block the binding of PD-L1 and or PD-L2, and inhibit the immunosuppressive effect of the PD-1 signalling pathway. In any of the compositions or methods disclosed herein, an anti-PD-1 antibody includes an antigen-binding portion or fragment that binds to the PD-1 receptor and exhibits the functional properties similar to those of whole Abs in inhibiting ligand binding and upregulating the immune system.

In certain embodiments, an anti-PD-1 antibody used in the methods can be replaced with another PD-1 or anti-PD-L1 antagonist. For example, because an anti-PD-L1 antibody prevents interaction between PD-1 and PD-L1, thereby exerting similar effects to the signalling pathway of PD-1, an anti-PD-L1 antibody can replace the use of an anti-PD-1 antibody in the methods disclosed herein. In any embodiment, suitable PD-L1 inhibitors include Imfinzi (durvalumab or MED14736), Tecentriq (atezolizumab or MPDL3280A), Bavencio (avelumab; MSB0010718C), MS-936559 (12A4 or MDX-1105) and CX-072.

Anti-CTLA-4 antibodies of the instant invention bind to human CTLA-4 so as to disrupt the interaction of CTLA-4 with a human B7 receptor. It will be understood that because the interaction of CTLA-4 with B7 transduces a signal leading to inactivation of T-cells bearing the CTLA-4 receptor, disruption of the interaction effectively induces, enhances or prolongs the activation of such T cells, thereby inducing, enhancing or prolonging an immune response.

Suitable CTLA-4 inhibitors that may be used in accordance with the invention include Yervoy (ipilimumab), Tremelimumab and AGEN 1884 or those disclosed in U.S. Pat. Nos. 6,984,720 and 7,605,238. Ipilimumab is a fully human, IgG1 monoclonal Ab that blocks the binding of CTLA-4 to its B7 ligands, thereby stimulating T cell activation. Tremelimumab is human IgG2 monoclonal anti-CTLA-4 antibody.

As will of course be appreciated, the CPI may be any other CPI known in the art, including any inhibitor of PD-1, PD-L1, CTLA-4, TIGIT, VISTA, LAG-3 or CD47.

Immune checkpoint inhibitors may be administered in the form of a pharmaceutical composition, including in combination with any pharmaceutically acceptable excipient, carrier and/or diluent described herein. Typically, the immune checkpoint inhibitor is administered in a formulation as is known in the art.

It will be appreciated that the disclosure herein is not limited to the use of the specific immune checkpoint inhibitors described herein, and includes use of the fusion protein or compositions of the invention, prior to, concomitantly with, or subsequent to administration of any immune checkpoint inhibitor, including non-antibody inhibitors.

It will be well within the purview of the skilled person to assess the therapeutic efficacy of the aforementioned methods. For instance, for the treatment of tumours, successful treatment may result in an inhibition of tumour growth by at least about 10%, by at least about 20%, by at least about 30%, by at least about 40%, by at least about 50%, by at least about 60%, by at least about 70%, by at least about 80%, or by at least about 90% or more, relative to untreated subjects. Alternatively, the treatments described herein may cause complete regression of the tumour mass. In other embodiments of the invention, tumour regression can be observed and continue for a period of at least about 10 days, at least about 20 days, at least about 30 days, at least about 40 days, at least about 50 days or at least about 60 days, at least about 70 days, at least about 80 days, at least about 90 days, at least about 100 days or longer.

The terms “subject” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

### Compositions

In some examples, a molecule as described herein can be administered orally, parenterally, by inhalation spray, adsorption, absorption, topically, rectally, nasally, bucally, vaginally, intraventricularly, via an implanted reservoir in dosage formulations containing conventional non-toxic pharmaceutically-acceptable carriers, or by any other convenient dosage form. The term “parenteral” as used herein includes subcutaneous, intravenous, intramuscular, intraperitoneal, intrathecal, intraventricular, intrasternal, and intracranial injection or infusion techniques.

Methods for preparing a molecule of the invention into a suitable form for administration to a subject (e.g. a pharmaceutical composition) are known in the art and include, for example, methods as described in Remington's Pharmaceutical Sciences (18th ed., Mack Publishing Co., Easton, Pa., 1990) and U.S. Pharmacopeia: National Formulary (Mack Publishing Company, Easton, Pa., 1984).

The pharmaceutical compositions of this invention are particularly useful for parenteral administration, such as intravenous administration or administration into a body cavity or lumen of an organ or joint. The compositions for administration will commonly comprise a solution of an antigen binding protein dissolved in a pharmaceutically acceptable carrier, for example an aqueous carrier. A variety of aqueous carriers can be used, e.g., buffered saline and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of an antigen binding site of the present invention in these formulations can vary widely, and will be selected primarily based on fluid volumes, viscosities, body weight and the like in accordance with the particular mode of administration selected and the patient's needs. Exemplary carriers include water, saline, Ringer's solution, dextrose solution, and 5% human serum albumin. Nonaqueous vehicles such as mixed oils and ethyl oleate may also be used. Liposomes may also be used as carriers. The vehicles may contain minor amounts of additives that enhance isotonicity and chemical stability, e.g., buffers and preservatives.

### Dosages and Timing of Administration

Suitable dosages of a molecule of the present invention will vary depending on the specific molecule, the condition to be treated and/or the subject being treated. It is within the ability of a skilled physician to determine a suitable dosage, e.g., by commencing with a sub-optimal dosage and incrementally modifying the dosage to determine an optimal or useful dosage. Alternatively, to determine an appropriate dosage for treatment/prophylaxis, data from the cell culture assays or animal studies are used, wherein a suitable dose is within a range of circulating concentrations that include the ED50 of the active compound with little or no toxicity. The dosage may vary within this range depending upon the dosage form employed and the route of administration utilized. A therapeutically/prophylactically effective dose can be estimated initially from cell culture assays. A dose may be formulated in animal models to achieve a circulating plasma concentration range that includes the IC50 (i.e., the concentration or amount of the compound which achieves a half-maximal inhibition of symptoms) as determined in cell culture. Such information can be used to more accurately determine useful doses in humans. Levels in plasma maybe measured, for example, by high performance liquid chromatography.

In some examples, a method of the present invention comprises administering a prophylactically or therapeutically effective amount of a protein described herein.

The term “therapeutically effective amount” is the quantity which, when administered to a subject in need of treatment, improves the prognosis and/or state of the subject and/or that reduces or inhibits one or more symptoms of a clinical condition described herein to a level that is below that observed and accepted as clinically diagnostic or clinically characteristic of that condition. The amount to be administered to a subject will depend on the particular characteristics of the condition to be treated, the type and stage of condition being treated, the mode of administration, and the characteristics of the subject, such as general health, other diseases, age, sex, genotype, and body weight. A person skilled in the art will be able to determine appropriate dosages depending on these and other factors. Accordingly, this term is not to be construed to limit the present invention to a specific quantity, e.g., weight or amount of protein(s), rather the present invention encompasses any amount of the antigen binding protein(s) sufficient to achieve the stated result in a subject.

As used herein, the term “prophylactically effective amount” shall be taken to mean a sufficient quantity of a protein to prevent or inhibit or delay the onset of one or more detectable symptoms of a clinical condition. The skilled artisan will be aware that such an amount will vary depending on, for example, the specific antigen binding protein(s) administered and/or the particular subject and/or the type or severity or level of condition and/or predisposition (genetic or otherwise) to the condition. Accordingly, this term is not to be construed to limit the present invention to a specific quantity, e.g., weight or amount of antigen binding protein(s), rather the present invention encompasses any amount of the antigen binding protein(s) sufficient to achieve the stated result in a subject.

In some embodiments, where the invention relates to combination therapy with a molecule of the invention followed by or concomitant with administration of an immune checkpoint inhibitor, the molecule of the invention and the immune checkpoint inhibitor are together administered to the subject in need thereof in a “therapeutically effective amount”. This therapeutically effective amount may comprise amounts of either or both the molecule of the invention or the immune checkpoint inhibitor that would by itself be less than a therapeutically effective amount. In some embodiments, the molecule of the invention is administered in an amount that is less than a therapeutically effective amount absent the immune checkpoint inhibitor. In some embodiments, the immune checkpoint inhibitor is administered in an amount that is less than a therapeutically effective amount absent the molecule of the invention. In some embodiments, the “therapeutically effective amount” for the combination of the molecule of the invention and the immune checkpoint inhibitor may be a synergistic amount. The synergistic amount may be synergistic relative to monotherapy with either molecule of the invention or the immune checkpoint inhibitor.

### Kits

The present invention additionally provides a kit comprising one or more of the following:
(i) a molecule of the invention or expression construct(s) encoding same; or
(ii) a pharmaceutical composition of the invention.

The kit may additionally comprise one or more therapeutic agents for administration to a subject, prior to or following administration of a molecule or composition of the invention.

Optionally a kit of the invention is packaged with instructions for use in a method described herein.

TABLE 1
Summary of amino acid and nucleotide sequences for PSMA-
binding antibodies for use in the invention

| | | SEQ ID | |
|---|---|---|---|
| Antibody ID | Region | NO: | Amino acid or nucleotide sequence |
| ANT4044 Variable | HCDR1 (protein) | 1 | EYTIH |
| Heavy chain | HCDR2 (protein) | 2 | NINPNNGGTTYNQKFED |
| | HCDR3 (protein) | 3 | GWNFDY |
| | VH (protein) | 4 | EVQLVQSGAEVKKPGASVKVSCKASGYTFTEYTIHW |
| | | | VRQAPGKGLEWIGNINPNNGGTTYNQKFEDRVTITV |
| | | | DKSTSTAYMELSSLRSEDTAVYYCAAGWNFDYWGQ |
| | | | GTTVTVSS |
| | HCDR1 (DNA) | 5 | GAATACACCATCCAC |
| | HCDR2 (DNA) | 6 | AACATTAATCCTAACAATGGTGGTACTACCTACAAC |
| | | | CAGAAGTTCGAGGAC |
| | HCDR3 (DNA) | 7 | GGTTGGAACTTTGACTAC |
| | VH (DNA) | 8 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACTGAATACACCATCC |
| | | | ACTGGGTGAGGCAGGCCCCTGGAAAGGGCCTTGA |
| | | | GTGGATTGGAAACATTAATCCTAACAATGGTGGTA |
| | | | CTACCTACAACCAGAAGTTCGAGGACAGAGTCACA |
| | | | ATCACTGTAGACAAGTCCACCAGCACAGCCTACAT |
| | | | GGAGCTCAGCAGCCTGAGATCTGAGGATACTGCA |
| | | | GTCTATTACTGTGCAGCTGGTTGGAACTTTGACTA |
| | | | CTGGGGCCAAGGCACCACGGTCACCGTCTCCTCA |
| | HFR1 (protein) | 9 | EVQLVQSGAEVKKPGASVKVSCKASGYTFT |
| | HFR2 (protein) | 10 | WVRQAPGKGLEWIG |
| | HFR3 (protein) | 11 | RVTITVDKSTSTAYMELSSLRSEDTAVYYCAA |
| | HFR4 (protein) | 12 | WGQGTTVTVSS |
| | HFR1 (DNA) | 12 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACT |
| | HFR2 (DNA) | 14 | TGGGTGAGGCAGGCCCCTGGAAAGGGCCTTGAGT |
| | | | GGATTGGA |
| | HFR3 (DNA) | 15 | AGAGTCACAATCACTGTAGACAAGTCCACCAGCAC |
| | | | AGCCTACATGGAGCTCAGCAGCCTGAGATCTGAG |
| | | | GATACTGCAGTCTATTACTGTGCAGCT |
| | HFR4 (DNA) | 16 | TGGGGCCAAGGCACCACGGTCACCGTCTCCTCA |
| ANT4044-A2 | HCDR1 (protein) | 17 | EYTIH |
| Variable Heavy | HCDR2 (protein) | 18 | NINPNNGGTTYNQKFED |
| chain | HCDR3 (protein) | 19 | YWLFDY |
| | VH (protein) | 20 | EVQLVQSGAEVKKPGASVKVSCKASGYTFTEYTIHW |
| | | | VRQAPGKGLEWIGNINPNNGGTTYNQKFEDRVTITV |
| | | | DKSTSTAYMELSSLRSEDTAVYYCAAYWLFDYWGQ |
| | | | GTTVTVSS |
| | HCDR1 (DNA) | 21 | GAATACACCATCCAC |
| | HCDR2 (DNA) | 22 | AACATTAATCCTAACAATGGTGGTACTACCTACAAC |
| | | | CAGAAGTTCGAGGAC |
| | HCDR3 (DNA) | 23 | TACTGGCTGTTCGACTAC |
| | VH (DNA) | 24 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACTGAATACACCATCC |
| | | | ACTGGGTGAGGCAGGCCCCTGGAAAGGGCCTTGA |
| | | | GTGGATTGGAAACATTAATCCTAACAATGGTGGTA |
| | | | CTACCTACAACCAGAAGTTCGAGGACAGAGTCACA |
| | | | ATCACTGTAGACAAGTCCACCAGCACAGCCTACAT |
| | | | GGAGCTCAGCAGCCTGAGATCTGAGGATACTGCA |
| | | | GTCTATTACTGTGCAGCTTACTGGCTGTTCGACTA |
| | | | CTGGGGCCAAGGCACCACGGTCACCGTCTCCTCA |
| | HFR1 (protein) | 25 | EVQLVQSGAEVKKPGASVKVSCKASGYTFT |
| | HFR2 (protein) | 26 | WVRQAPGKGLEWIG |
| | HFR3 (protein) | 27 | RVTITVDKSTSTAYMELSSLRSEDTAVYYCAA |
| | HFR4 (protein) | 28 | WGQGTTVTVSS |
| | HFR1 (DNA) | 29 | GAGGTCCAGCTGGTGCAGTCTGGAGCTGAGGTGA |
| | | | AGAAGCCTGGGGCCTCAGTGAAGGTCTCCTGCAA |
| | | | GGCTTCTGGATACACATTCACT |
| | HFR2 (DNA) | 30 | TGGGTGAGGCAGGCCCCTGGAAAGGGCCTTGAGT |
| | | | GGATTGGA |
| | HFR3 (DNA) | 31 | AGAGTCACAATCACTGTAGACAAGTCCACCAGCAC |
| | | | AGCCTACATGGAGCTCAGCAGCCTGAGATCTGAG |
| | | | GATACTGCAGTCTATTACTGTGCAGCT |
| | HFR4 (DNA) | 32 | TGGGGCCAAGGCACCACGGTCACCGTCTCCTCA |
| ANT4044/ | LCDR1 (protein) | 33 | KASQDVGTAVD |
| ANT4044-A2 | LCDR2 (protein) | 34 | WASTRHT |
| Variable Light | LCDR3 (protein) | 35 | QQYNSYPLT |
| chain | VL (protein) | 36 | DIQMTQSPSTLSASVGDRVTITCKASQDVGTAVDWY |
| | | | QQKPGQAPKLLIYWASTRHTGVPDRFSGSGSGTDFT |
| | | | LTISRLQPEDFAVYYCQQYNSYPLTFGQGTKVDIK |
| | LCDR1 (DNA) | 37 | AAGGCCAGTCAGGATGTGGGTACTGCTGTAGAC |
| | LCDR2 (DNA) | 38 | TGGGCATCCACCCGGCACACT |
| | LCDR3 (DNA) | 39 | CAGCAATATAACAGCTATCCTCTCACG |
| | VL (DNA) | 40 | GACATTCAGATGACCCAGTCTCCCAGCACCCTGTC |
| | | | CGCATCAGTAGGAGACAGGGTCACCATCACTTGCA |
| | | | AGGCCAGTCAGGATGTGGGTACTGCTGTAGACTG |
| | | | GTATCAACAGAAACCAGGGCAAGCTCCTAAACTAC |
| | | | TGATTTACTGGGCATCCACCCGGCACACTGGAGTC |
| | | | CCTGATCGCTTCAGCGGCAGTGGATCTGGGACAG |
| | | | ATTTCACTCTCACCATCAGCAGACTGCAGCCTGAA |
| | | | GACTTTGCAGTTTATTACTGTCAGCAATATAACAGC |
| | | | TATCCTCTCACGTTCGGCCAGGGGACCAAGGTGG |
| | | | ATATCAAA |
| | LFR1 (protein) | 41 | DIQMTQSPSTLSASVGDRVTITC |
| | LFR2 (protein) | 42 | WYQQKPGQAPKLLIY |
| | LFR3 (protein) | 43 | GVPDRFSGSGSGTDFTLTISRLQPEDFAVYYC |
| | LFR4 (protein) | 44 | FGQGTKVDIK |
| | LFR1 (DNA) | 45 | GACATTCAGATGACCCAGTCTCCCAGCACCCTGTC |
| | | | CGCATCAGTAGGAGACAGGGTCACCATCACTTGC |
| | LFR2 (DNA) | 46 | TGGTATCAACAGAAACCAGGGCAAGCTCCTAAACT |
| | | | ACTGATTTAC |
| | LFR3 (DNA) | 47 | GGAGTCCCTGATCGCTTCAGCGGCAGTGGATCTG |
| | | | GGACAGATTTCACTCTCACCATCAGCAGACTGCAG |
| | | | CCTGAAGACTTTGCAGTTTATTACTGT |
| | LFR4 (DNA) | 48 | TTCGGCCAGGGGACCAAGGTGGATATCAAA |
| ANT4044/ANT4044- | IgG1 HC | 235 | ASTKGPSVFPLAPSSKSTSGGTAALGCLVKDYFPEP |
| A2_unmodified | (protein) | | VTVSWNSGALTSGVHTFPAVLQSSGLYSLSSVVTVP |
| human IgG1 | | | SSSLGTQTYICNVNHKPSNTKVDKRVEPKSCDKTHT |
| heavy chain | | | CPPCPAPELLGGPSVFLFPPKPKDTLMISRTPEVTCV |
| constant region | | | VVDVSHEDPEVKFNWYVDGVEVHNAKTKPREEQYN |
| | | | STYRVVSVLTVLHQDWLNGKEYKCKVSNKALPAPIEK |
| | | | TISKAKGQPREPQVYTLPPSREEMTKNQVSLTCLVK |
| | | | GFYPSDIAVEWESNGQPENNYKTTPPVLDSDGSFFL |
| | | | YSKLTVDKSRWQQGNVFSCSVMHEALHNHYTQKSL |
| | | | SLSPGK |
| ANT4044/ANT4044- | IgG1 H310A | 236 | ASTKGPSVFPLAPSSKSTSGGTAALGCLVKDYFPEP |
| A2 modified | H435Q HC | | VTVSWNSGALTSGVHTFPAVLQSSGLYSLSSVVTVP |
| human IgG1 | (protein) | | SSSLGTQTYICNVNHKPSNTKVDKRVEPKSCDKTHT |
| heavy chain | | | CPPCPAPELLGGPSVFLFPPKPKDTLMISRTPEVTCV |
| constant region | | | VVDVSHEDPEVKFNWYVDGVEVHNAKTKPREEQYN |
| | | | STYRVVSVLTVLAQDWLNGKEYKCKVSNKALPAPIEK |
| | | | TISKAKGQPREPQVYTLPPSREEMTKNQVSLTCLVK |
| | | | GFYPSDIAVEWESNGQPENNYKTTPPVLDSDGSFFL |
| | | | YSKLTVDKSRWQQGNVFSCSVMHEALHNQYTQKSL |
| | | | SLSPGK |
| ANT4044/ANT4044- | IgG4 S228P | 237 | ASTKGPSVFPLAPCSRSTSESTAALGCLVKDYFPEPV |
| A2_modified | L235E H310A | | TVSWNSGALTSGVHTFPAVLQSSGLYSLSSVVTVPS |
| human IgG4 | H435Q HC | | SSLGTKTYTCNVDHKPSNTKVDKRVESKYGPPCPPC |
| constant chain | (protein) | | PAPEFEGGPSVFLFPPKPKDTLMISRTPEVTCVVVDV |
| region | | | SQEDPEVQFNWYVDGVEVHNAKTKPREEQFNSTYR |
| | | | VVSVLTVLAQDWLNGKEYKCKVSNKGLPSSIEKTISK |
| | | | AKGQPREPQVYTLPPSQEEMTKNQVSLTCLVKGFYP |
| | | | SDIAVEWESNGQPENNYKTTPPVLDSDGSFFLYSRL |
| | | | TVDKSRWQEGNVFSCSVMHEALHNQYTQKSLSLSL |
| | | | GK |
| ANT4044/ANT4044- | Human κ LC | 238 | RTVAAPSVFIFPPSDEQLKSGTASVVCLLNNFYPREA |
| A2 kappa light | constant region | | KVQWKVDNALQSGNSQESVTEQDSKDSTYSLSSTLT |
| chain constant | | | LSKADYEKHKVYACEVTHQGLSSPVTKSFNRGEC |
| region | | | |
| ANT4044 | FcRn-null, IgG1 | 239 | EVQLVQSGAEVKKPGASVKVSCKASGYTFTEYTIHW |
| RADmAb IgG1 | allotype G1m(3) | | VRQAPGKGLEWIGNINPNNGGTTYNQKFEDRVTITV |
| heavy chain | H310A H435Q | | DKSTSTAYMELSSLRSEDTAVYYCAAGWNFDYWGQ |
| (HuX592r) | | | GTTVTVSSASTKGPSVFPLAPSSKSTSGGTAALGCLV |
| | | | KDYFPEPVTVSWNSGALTSGVHTFPAVLQSSGLYSL |
| | | | SSVVTVPSSSLGTQTYICNVNHKPSNTKVDKRVEPKS |
| | | | CDKTHTCPPCPAPELLGGPSVFLFPPKPKDTLMISRT |
| | | | PEVTCVVVDVSHEDPEVKFNWYVDGVEVHNAKTKP |
| | | | REEQYNSTYRVVSVLTVLAQDWLNGKEYKCKVSNK |
| | | | ALPAPIEKTISKAKGQPREPQVYTLPPSREEMTKNQV |
| | | | SLTCLVKGFYPSDIAVEWESNGQPENNYKTTPPVLD |
| | | | SDGSFFLYSKLTVDKSRWQQGNVFSCSVMHEALHN |
| | | | QYTQKSLSLSPGK |
| ANT4044 | FcRn + FcRγ-null, | 240 | EVQLVQSGAEVKKPGASVKVSCKASGYTFTEYTIHW |
| RADmAb IgG4 | IgG4 S228P | | VRQAPGKGLEWIGNINPNNGGTTYNQKFEDRVTITV |
| heavy chain | L235E H310A | | DKSTSTAYMELSSLRSEDTAVYYCAAGWNFDYWGQ |
| | H435Q | | GTTVTVSSASTKGPSVFPLAPCSRSTSESTAALGCLV |
| | | | KDYFPEPVTVSWNSGALTSGVHTFPAVLQSSGLYSL |
| | | | SSVVTVPSSSLGTKTYTCNVDHKPSNTKVDKRVESK |
| | | | YGPPCPPCPAPEFEGGPSVFLFPPKPKDTLMISRTPE |
| | | | VTCVVVDVSQEDPEVQFNWYVDGVEVHNAKTKPRE |
| | | | EQFNSTYRVVSVLTVLAQDWLNGKEYKCKVSNKGLP |
| | | | SSIEKTISKAKGQPREPQVYTLPPSQEEMTKNQVSLT |
| | | | CLVKGFYPSDIAVEWESNGQPENNYKTTPPVLDSDG |
| | | | SFFLYSRLTVDKSRWQEGNVFSCSVMHEALHNQYT |
| | | | QKSLSLSLGK |
| ANT4044-A2 | FcRn-null, IgG1 | 241 | EVQLVQSGAEVKKPGASVKVSCKASGYTFTEYTIHW |
| RADmAb IgG1 | H310A H435Q | | VRQAPGKGLEWIGNINPNNGGTTYNQKFEDRVTITV |
| heavy chain | | | DKSTSTAYMELSSLRSEDTAVYYCAAYWLFDYWGQ |
| | | | GTTVTVSSASTKGPSVFPLAPSSKSTSGGTAALGCLV |
| | | | KDYFPEPVTVSWNSGALTSGVHTFPAVLQSSGLYSL |
| | | | SSVVTVPSSSLGTQTYICNVNHKPSNTKVDKRVEPKS |
| | | | CDKTHTCPPCPAPELLGGPSVFLFPPKPKDTLMISRT |
| | | | PEVTCVVVDVSHEDPEVKFNWYVDGVEVHNAKTKP |
| | | | REEQYNSTYRVVSVLTVLAQDWLNGKEYKCKVSNK |
| | | | ALPAPIEKTISKAKGQPREPQVYTLPPSREEMTKNQV |
| | | | SLTCLVKGFYPSDIAVEWESNGQPENNYKTTPPVLD |
| | | | SDGSFFLYSKLTVDKSRWQQGNVFSCSVMHEALHN |
| | | | QYTQKSLSLSPGK |
| ANT4044-A2 | FcRn + FcRγ-null | 242 | EVQLVQSGAEVKKPGASVKVSCKASGYTFTEYTIHW |
| RADmAb IgG4 | IgG4 S228P | | VRQAPGKGLEWIGNINPNNGGTTYNQKFEDRVTITV |
| heavy chain | L235E H310A | | DKSTSTAYMELSSLRSEDTAVYYCAAYWLFDYWGQ |
| | H435Q | | GTTVTVSSASTKGPSVFPLAPCSRSTSESTAALGCLV |
| | | | KDYFPEPVTVSWNSGALTSGVHTFPAVLQSSGLYSL |
| | | | SSVVTVPSSSLGTKTYTCNVDHKPSNTKVDKRVESK |
| | | | YGPPCPPCPAPEFEGGPSVFLFPPKPKDTLMISRTPE |
| | | | VTCVVVDVSQEDPEVQFNWYVDGVEVHNAKTKPRE |
| | | | EQFNSTYRVVSVLTVLAQDWLNGKEYKCKVSNKGLP |
| | | | SSIEKTISKAKGQPREPQVYTLPPSQEEMTKNQVSLT |
| | | | CLVKGFYPSDIAVEWESNGQPENNYKTTPPVLDSDG |
| | | | SFFLYSRLTVDKSRWQEGNVFSCSVMHEALHNQYT |
| | | | QKSLSLSLGK |
| ANT4044- | Vk Light chain | 243 | DIQMTQSPSTLSASVGDRVTITCKASQDVGTAVDWY |
| ANT4044-A2 Vk | | | QQKPGQAPKLLIYWASTRHTGVPDRFSGSGSGTDFT |
| Light chain | | | LTISRLQPEDFAVYYCQQYNSYPLTFGQGTKVDIKRT |
| | | | VAAPSVFIFPPSDEQLKSGTASVVCLLNNFYPREAKV |
| | | | QWKVDNALQSGNSQESVTEQDSKDSTYSLSSTLTLS |
| | | | KADYEKHKVYACEVTHQGLSSPVTKSFNRGEC |
| Anti-PSMA | alternative heavy | 244 | EVQLVQSGPEVKKPGATVKISCKTSGYTFTEYTIHWV |
| | chain variable | | KQAPGKGLEWIGNINPNNGGTTYNQKFEDKATLTVD |
| | sequence | | KSTDTAYMELSSLRSEDTAVYYCAAGWNFDYWGQG |
| | | | TLLTVSS |
| Anti-PSMA | alternative light | 245 | DIQMTQSPSSLSTSVGDRVTLTCKASQDVGTAVDWY |
| | chain variable | | QQKPGPSPKLLIYWASTRHTGIPSRFSGSGSGTDFTL |
| | sequence | | TISSLQPEDFADYYCQQYNSYPLTFGPGTKVDIK |

TABLE 2
Summary of amino acid and nucleotide sequences for CAIX-
binding antibodies for use in the invention

| | | SEQ ID | |
|---|---|---|---|
| Antibody ID | Region | NO: | Amino acid or nucleotide sequence |
| Girentuximab | HCDR1 (protein) | 49 | NYYMS |
| chimeric VH0 | HCDR2 (protein) | 50 | AINSDGGITYYLDTVKG |
| Variable Heavy | HCDR3 (protein) | 51 | HRSGYFSMDY |
| chain | VH (protein) | 52 | DVKLVESGGGLVKLGGSLKLSCAASGFTFSNYYMSWV |
| | | | RQTPEKRLELVAAINSDGGITYYLDTVKGRFTISRDNAK |
| | | | NTLYLQMSSLKSEDTALFYCARHRSGYFSMDYWGQG |
| | | | TSVTVSS |
| | HCDR1 (DNA) | 53 | AACTATTACATGTCT |
| | HCDR2 (DNA) | 54 | GCCATTAATAGTGATGGTGGTATCACCTACTATCTAG |
| | | | ACACTGTGAAGGGC |
| | HCDR3 (DNA) | 55 | CACCGCTCGGGCTACTTTTCTATGGACTAC |
| | VH (DNA) | 56 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGTAACTATTACATGTCTTGG |
| | | | GTTCGCCAGACTCCAGAGAAGAGGCTGGAGTTGGT |
| | | | CGCAGCCATTAATAGTGATGGTGGTATCACCTACTA |
| | | | TCTAGACACTGTGAAGGGCCGATTCACCATTTCAAG |
| | | | AGACAATGCCAAGAACACCCTGTACCTGCAAATGAG |
| | | | CAGTCTGAAGTCTGAGGACACAGCCTTGTTTTACTG |
| | | | TGCAAGACACCGCTCGGGCTACTTTTCTATGGACTA |
| | | | CTGGGGTCAAGGAACCTCAGTCACCGTCTCCTCA |
| | HFR1 (protein) | 57 | DVKLVESGGGLVKLGGSLKLSCAASGFTFS |
| | HFR2 (protein) | 58 | WVRQTPEKRLELVA |
| | HFR3 (protein) | 59 | RFTISRDNAKNTLYLQMSSLKSEDTALFYCAR |
| | HFR4 (protein) | 60 | WGQGTSVTVSS |
| | HFR1 (DNA) | 61 | GACGTGAAGCTCGTGGAGTCTGGGGGAGGCTTAGT |
| | | | GAAGCTTGGAGGATCCCTGAAACTCTCCTGTGCAGC |
| | | | CTCTGGATTCACTTTCAGT |
| | HFR2 (DNA) | 62 | TGGGTTCGCCAGACTCCAGAGAAGAGGCTGGAGTT |
| | | | GGTCGCA |
| | HFR3 (DNA) | 63 | CGATTCACCATTTCAAGAGACAATGCCAAGAACACC |
| | | | CTGTACCTGCAAATGAGCAGTCTGAAGTCTGAGGAC |
| | | | ACAGCCTTGTTTTACTGTGCAAGA |
| | HFR4 (DNA) | 64 | TGGGGTCAAGGAACCTCAGTCACCGTCTCCTCA |
| Girentuximab | HCDR1 (protein) | 65 | NYYMS |
| humanised VH1 | HCDR2 (protein) | 66 | AINSDGGITYYLDTVKG |
| Variable Heavy | HCDR3 (protein) | 67 | HRSGYFSMDY |
| chain | VH (protein) | 68 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMSSLKSEDTALYYCARHRSGYFSMDYWG |
| | | | QGTSVTVSS |
| | HCDR1 (DNA) | 69 | AACTACTACATGAGC |
| | HCDR2 (DNA) | 70 | GCCATTAACAGTGACGGTGGCATCACCTACTACCTG |
| | | | GACACCGTGAAGGGC |
| | HCDR3 (DNA) | 71 | CACAGGAGCGGCTACTTCTCTATGGACTAC |
| | VH (DNA) | 72 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAGCAGCCTGAAGAGCGAGGACACGGCCCTGTATT |
| | | | ACTGTGCGAGACACAGGAGCGGCTACTTCTCTATGG |
| | | | ACTACTGGGGCCAGGGCACCAGCGTCACTGTCTCC |
| | | | TCA |
| | HFR1 (protein) | 73 | DVKLVESGGGLVKPGGSLRLSCAASGFTFS |
| | HFR2 (protein) | 74 | WVRQAPGKGLELVA |
| | HFR3 (protein) | 75 | RFTISRDNAKNTLYLQMSSLKSEDTALYYCAR |
| | HFR4 (protein) | 76 | WGQGTSVTVSS |
| | HFR1 (DNA) | 77 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 78 | TGGGTGCGCCAGGCTCCAGGGAAGGGGCTGGAGC |
| | | | TGGTTGCC |
| | HFR3 (DNA) | 79 | CGATTCACCATCTCCAGGGACAACGCCAAGAACACC |
| | | | CTGTATCTGCAAATGAGCAGCCTGAAGAGCGAGGA |
| | | | CACGGCCCTGTATTACTGTGCGAGA |
| | HFR4 (DNA) | 80 | TGGGGCCAGGGCACCAGCGTCACTGTCTCCTCA |
| Girentuximab | HCDR1 (protein) | 81 | NYYMS |
| humanised VH3 | HCDR2 (protein) | 82 | AINSDGGITYYLDTVKG |
| Variable Heavy | HCDR3 (protein) | 83 | HRSGYFSMDY |
| chain | VH (protein) | 84 | DVKLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 85 | AACTACTACATGAGC |
| | | | ACACCGTGAAGGGC |
| | HCDR3 (DNA) | 87 | CACAGGAGCGGCTACTTCTCTATGGACTAC |
| | VH (DNA) | 88 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 89 | DVKLVESGGGLVKPGGSLRLSCAASGFTFS |
| | HFR2 (protein) | 90 | WVRQAPGKGLELVA |
| | HFR3 (protein) | 91 | RFTISRDNAKNTLYLQMNSLRAEDTALYYCAR |
| | HFR4 (protein) | 92 | WGQGTLVTVSS |
| | HFR1 (DNA) | 93 | GACGTGAAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 94 | TGGGTGCGCCAGGCTCCAGGGAAGGGGCTGGAGC |
| | | | TGGTTGCC |
| | HFR3 (DNA) | 95 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 96 | TGGGGCCAGGGCACCCTGGTCACTGTCTCCTCA |
| Girentuximab | HCDR1 (protein) | 97 | NYYMS |
| humanised VH4 | HCDR2 (protein) | 98 | AINSDGGITYYLDTVKG |
| Variable Heavy | HCDR3 (protein) | 99 | HRSGYFSMDY |
| chain | VH (protein) | 100 | EVQLVESGGGLVKPGGSLRLSCAASGFTFSNYYMSW |
| | | | VRQAPGKGLELVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 101 | AACTACTACATGAGC |
| | HCDR2 (DNA) | 102 | GCCATTAACAGTGACGGTGGCATCACCTACTACCTG |
| | | | GACACCGTGAAGGGC |
| | HCDR3 (DNA) | 103 | CACAGGAGCGGCTACTTCTCTATGGACTAC |
| | VH (DNA) | 104 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGCT |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 105 | EVQLVESGGGLVKPGGSLRLSCAASGFTFS |
| | HFR2 (protein) | 106 | WVRQAPGKGLELVA |
| | HFR3 (protein) | 107 | RFTISRDNAKNTLYLQMNSLRAEDTALYYCAR |
| | HFR4 (protein) | 108 | WGQGTLVTVSS |
| | HFR1 (DNA) | 109 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 110 | TGGGTGCGCCAGGCTCCAGGGAAGGGGCTGGAGC |
| | | | TGGTTGCC |
| | HFR3 (DNA) | 111 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 112 | TGGGGCCAGGGCACCCTGGTCACTGTCTCCTCA |
| Girentuximab | HCDR1 (protein) | 113 | NYYMS |
| humanised VH5 | HCDR2 (protein) | 114 | AINSDGGITYYLDTVKG |
| Variable Heavy | HCDR3 (protein) | 115 | HRSGYFSMDY |
| chain | VH (protein) | 116 | EVQLVESGGGLVKPGGSLRLSCAASGFTFSNYYMSW |
| | | | VRQAPGKGLEWVAAINSDGGITYYLDTVKGRFTISRDN |
| | | | AKNTLYLQMNSLRAEDTALYYCARHRSGYFSMDYWG |
| | | | QGTLVTVSS |
| | HCDR1 (DNA) | 117 | AACTACTACATGAGC |
| | HCDR2 (DNA) | 118 | GCCATTAACAGTGACGGTGGCATCACCTACTACCTG |
| | | | GACACCGTGAAGGGC |
| | HCDR3 (DNA) | 119 | CACAGGAGCGGCTACTTCTCTATGGACTAC |
| | VH (DNA) | 120 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGTAACTACTACATGAGCT |
| | | | GGGTGCGCCAGGCTCCAGGGAAGGGGCTGGAGTG |
| | | | GGTTGCCGCCATTAACAGTGACGGTGGCATCACCTA |
| | | | CTACCTGGACACCGTGAAGGGCCGATTCACCATCTC |
| | | | CAGGGACAACGCCAAGAACACCCTGTATCTGCAAAT |
| | | | GAACAGCCTGAGGGCCGAGGACACGGCCCTGTATT |
| | | | ACTGTGCGAGACACAGGAGCGGCTACTTCTCTATGG |
| | | | ACTACTGGGGCCAGGGCACCCTGGTCACTGTCTCC |
| | | | TCA |
| | HFR1 (protein) | 121 | EVQLVESGGGLVKPGGSLRLSCAASGFTFS |
| | HFR2 (protein) | 122 | WVRQAPGKGLEWVA |
| | HFR3 (protein) | 123 | RFTISRDNAKNTLYLQMNSLRAEDTALYYCAR |
| | HFR4 (protein) | 124 | WGQGTLVTVSS |
| | HFR1 (DNA) | 125 | GAGGTGCAGCTGGTGGAGTCTGGGGGAGGCTTGGT |
| | | | CAAGCCTGGAGGATCCCTGAGACTCTCCTGTGCAG |
| | | | CCTCTGGATTCACCTTCAGT |
| | HFR2 (DNA) | 126 | TGGGTGCGCCAGGCTCCAGGGAAGGGGCTGGAGT |
| | | | GGGTTGCC |
| | HFR3 (DNA) | 127 | CGATTCACCATCTCCAGGGACAACGCCAAGAACACC |
| | | | CTGTATCTGCAAATGAACAGCCTGAGGGCCGAGGA |
| | | | CACGGCCCTGTATTACTGTGCGAGA |
| | HFR4 (DNA) | 128 | TGGGGCCAGGGCACCCTGGTCACTGTCTCCTCA |
| Girentuximab | LCDR1 (protein) | 129 | KASQNVVSAVA |
| Chimeric Vk0 | LCDR2 (protein) | 130 | SASNRYT |
| Variable light | LCDR3 (protein) | 131 | QQYSNYPWT |
| chain | VL (protein) | 132 | DIVMTQSQRFMSTTVGDRVSITCKASQNVVSAVAWYQ |
| | | | QKPGQSPKLLIYSASNRYTGVPDRFTGSGSGTDFTLTI |
| | | | SNMQSEDLADFFCQQYSNYPWTFGGGTKLEIK |
| | LCDR1 (DNA) | 133 | AAGGCCAGTCAGAATGTGGTTTCTGCTGTTGCC |
| | LCDR2 (DNA) | 134 | TCAGCATCCAATCGGTACACT |
| | LCDR3 (DNA) | 135 | CAACAATATAGCAACTATCCGTGGACG |
| | VL (DNA) | 136 | GACATTGTGATGACCCAGTCTCAAAGATTCATGTCC |
| | | | ACAACAGTAGGAGACAGGGTCAGCATCACCTGCAA |
| | | | GGCCAGTCAGAATGTGGTTTCTGCTGTTGCCTGGTA |
| | | | TCAACAGAAACCAGGACAATCTCCTAAACTACTGATT |
| | | | TACTCAGCATCCAATCGGTACACTGGAGTCCCTGAT |
| | | | CGCTTCACAGGCAGTGGATCTGGGACAGATTTCACT |
| | | | CTCACCATTAGCAATATGCAGTCTGAAGACCTGGCT |
| | | | GATTTTTTCTGTCAACAATATAGCAACTATCCGTGGA |
| | | | CGTTCGGTGGAGGCACCAAGCTGGAAATCAAA |
| | LFR1 (protein) | 137 | DIVMTQSQRFMSTTVGDRVSITC |
| | LFR2 (protein) | 138 | WYQQKPGQSPKLLIY |
| | LFR3 (protein) | 139 | GVPDRFTGSGSGTDFTLTISNMQSEDLADFFC |
| | LFR4 (protein) | 140 | FGGGTKLEIK |
| | LFR1 (DNA) | 141 | GACATTGTGATGACCCAGTCTCAAAGATTCATGTCC |
| | | | ACAACAGTAGGAGACAGGGTCAGCATCACCTGC |
| | LFR2 (DNA) | 142 | TGGTATCAACAGAAACCAGGACAATCTCCTAAACTA |
| | | | CTGATTTAC |
| | LFR3 (DNA) | 143 | GGAGTCCCTGATCGCTTCACAGGCAGTGGATCTGG |
| | | | GACAGATTTCACTCTCACCATTAGCAATATGCAGTCT |
| | | | GAAGACCTGGCTGATTTTTTCTGT |
| | LFR4 (DNA) | 144 | TTCGGTGGAGGCACCAAGCTGGAAATCAAA |
| Girentuximab | LCDR1 (protein) | 145 | KASQNVVSAVA |
| Humanised Vk1 | LCDR2 (protein) | 146 | SASNRYT |
| Variable light | LCDR3 (protein) | 147 | QQYSNYPWT |
| chain | VL (protein) | 148 | DIVMTQSPSFLSASVGDRVTITCKASQNVVSAVAWYQ |
| | | | QKPGQAPKLLIYSASNRYTGVPDRFTGSGSGTDFTLTI |
| | | | SSLQSEDLADYFCQQYSNYPWTFGGGTKVEIK |
| | LCDR1 (DNA) | 149 | AAGGCAAGTCAGAACGTGGTGAGTGCTGTGGCC |
| | LCDR2 (DNA) | 150 | AGCGCCTCCAACAGGTACACC |
| | LCDR3 (DNA) | 151 | CAACAGTACAGCAATTACCCTTGGACG |
| | VL (DNA) | 152 | GACATCGTGATGACCCAGTCTCCATCCTTCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAAGCTCCTGAT |
| | | | CTATAGCGCCTCCAACAGGTACACCGGGGTCCCAG |
| | | | ACAGGTTCACCGGCAGTGGATCTGGGACAGATTTCA |
| | | | CTCTCACCATCAGCAGCCTGCAGAGCGAAGATCTG |
| | | | GCAGACTATTTCTGTCAACAGTACAGCAATTACCCTT |
| | | | GGACGTTCGGGGGGGGGACCAAGGTGGAAATCAAA |
| | LFR1 (protein) | 153 | DIVMTQSPSFLSASVGDRVTITC |
| | LFR2 (protein) | 154 | WYQQKPGQAPKLLIY |
| | LFR3 (protein) | 155 | GVPDRFTGSGSGTDFTLTISSLQSEDLADYFC |
| | LFR4 (protein) | 156 | FGGGTKVEIK |
| | LFR1 (DNA) | 157 | GACATCGTGATGACCCAGTCTCCATCCTTCCTGTCT |
| | | | GCATCTGTAGGAGACAGAGTCACCATCACTTGC |
| | LFR2 (DNA) | 158 | TGGTATCAGCAGAAACCAGGGCAGGCTCCTAAGCT |
| | | | CCTGATCTAT |
| | LFR3 (DNA) | 159 | GGGGTCCCAGACAGGTTCACCGGCAGTGGATCTGG |
| | | | GACAGATTTCACTCTCACCATCAGCAGCCTGCAGAG |
| | | | CGAAGATCTGGCAGACTATTTCTGT |
| | LFR4 (DNA) | 160 | TTCGGCGGCGGGACCAAGGTGGAAATCAAA |
| Girentuximab | LCDR1 (protein) | 161 | KASQNVVSAVA |
| Humanised Vk2 | LCDR2 (protein) | 162 | SASNRYT |
| Variable light | LCDR3 (protein) | 163 | QQYSNYPWT |
| chain | VL (protein) | 164 | DIVMTQSPSSLSASVGDRVTITCKASQNVVSAVAWYQ |
| | | | QKPGQAPRLLIYSASNRYTGVPDRFTGSGSGTDFTLTI |
| | | | SSLQAEDLADYFCQQYSNYPWTFGGGTKVEIK |
| | LCDR1 (DNA) | 165 | AAGGCAAGTCAGAACGTGGTGAGTGCTGTGGCC |
| | LCDR2 (DNA) | 166 | AGCGCCTCCAACAGGTACACC |
| | LCDR3 (DNA) | 167 | CAACAGTACAGCAATTACCCTTGGACG |
| | VL (DNA) | 168 | GACATCGTGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCACCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 (protein) | 169 | DIVMTQSPSSLSASVGDRVTITC |
| | LFR2 (protein) | 170 | WYQQKPGQAPRLLIY |
| | LFR3 (protein) | 171 | GVPDRFTGSGSGTDFTLTISSLQAEDLADYFC |
| | LFR4 (protein) | 172 | FGGGTKVEIK |
| | LFR1 (DNA) | 173 | GACATCGTGATGACCCAGTCTCCATCCAGCCTGTCT |
| | | | GCATCTGTAGGAGACAGAGTCACCATCACTTGC |
| | LFR2 (DNA) | 174 | TGGTATCAGCAGAAACCAGGGCAGGCTCCTAGGCT |
| | | | CCTGATCTAT |
| | LFR3 (DNA) | 175 | GGGGTCCCAGACAGGTTCACCGGCAGTGGATCTGG |
| | | | GACAGATTTCACTCTCACCATCAGCAGCCTGCAGGC |
| | | | CGAAGATCTGGCAGACTATTTCTGT |
| | LFR4 (DNA) | 176 | TTCGGCGGCGGGACCAAGGTGGAAATCAAA |
| Girentuximab | LCDR1 (protein) | 177 | KASQNVVSAVA |
| Humanised Vk3 | LCDR2 (protein) | 178 | SASNRYT |
| Variable light | LCDR3 (protein) | 179 | QQYSNYPWT |
| chain | VL (protein) | 180 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYFCQQYSNYPWTFGGGTKVEIK |
| | LCDR1 (DNA) | 181 | AAGGCAAGTCAGAACGTGGTGAGTGCTGTGGCC |
| | LCDR2 (DNA) | 182 | AGCGCCTCCAACAGGTACACC |
| | LCDR3 (DNA) | 183 | CAACAGTACAGCAATTACCCTTGGACG |
| | VL (DNA) | 184 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTTCTGTCAACAGTACAGCAATTACCC |
| | | | TTGGACGTTCGGCGGCGGGACCAAGGTGGAAATCA |
| | | | AA |
| | LFR1 (protein) | 185 | DIQMTQSPSSLSASVGDRVTITC |
| | LFR2 (protein) | 186 | WYQQKPGQAPRLLIY |
| | LFR3 (protein) | 187 | GVPDRFSGSGSGTDFTLTISSLQAEDLADYFC |
| | LFR4 (protein) | 188 | FGGGTKVEIK |
| | LFR1 (DNA) | 189 | GACATCCAGATGACCCAGTCTCCATCCAGCCTGTCT |
| | | | GCATCTGTAGGAGACAGAGTCACCATCACTTGC |
| | LFR2 (DNA) | 190 | TGGTATCAGCAGAAACCAGGGCAGGCTCCTAGGCT |
| | | | CCTGATCTAT |
| | LFR3 (DNA) | 191 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTTCTGT |
| | LFR4 (DNA) | 192 | TTCGGCGGCGGGACCAAGGTGGAAATCAAA |
| Girentuximab | LCDR1 (protein) | 193 | KASQNVVSAVA |
| Humanised Vk4 | LCDR2 (protein) | 194 | SASNRYT |
| Variable light | LCDR3 (protein) | 195 | QQYSNYPWT |
| chain | VL (protein) | 196 | DIQMTQSPSSLSASVGDRVTITCKASQNVVSAVAWYQ |
| | | | QKPGQAPRLLIYSASNRYTGVPDRFSGSGSGTDFTLTI |
| | | | SSLQAEDLADYYCQQYSNYPWTFGGGTKVEIK |
| | LCDR1 (DNA) | 197 | AAGGCAAGTCAGAACGTGGTGAGTGCTGTGGCC |
| | LCDR2 (DNA) | 198 | AGCGCCTCCAACAGGTACACC |
| | LCDR3 (DNA) | 199 | CAACAGTACAGCAATTACCCTTGGACG |
| | VL (DNA) | 200 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGCTCCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 (protein) | 201 | DIQMTQSPSSLSASVGDRVTITC |
| | LFR2 (protein) | 202 | WYQQKPGQAPRLLIY |
| | LFR3 (protein) | 203 | GVPDRFSGSGSGTDFTLTISSLQAEDLADYYC |
| | LFR4 (protein) | 204 | FGGGTKVEIK |
| | LFR1 (DNA) | 205 | GACATCCAGATGACCCAGTCTCCATCCAGCCTGTCT |
| | | | GCATCTGTAGGAGACAGAGTCACCATCACTTGC |
| | LFR2 (DNA) | 206 | TGGTATCAGCAGAAACCAGGGCAGGCTCCTAGGCT |
| | | | CCTGATCTAT |
| | LFR3 (DNA) | 207 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 (DNA) | 208 | TTCGGCGGCGGGACCAAGGTGGAAATCAAA |
| Girentuximab | LCDR1 (protein) | 209 | KASQNVVSAVA |
| Humanised Vk5 | LCDR2 (protein) | 210 | SASNRYT |
| Variable light | LCDR3 (protein) | 211 | QQYSNYPWT |
| chain | VL (protein) | 212 | DIQMTQSPSSLSASVGDRVTITCKASQNVVSAVAWYQ |
| | | | QKPGQAPRRLIYSASNRYTGVPDRFSGSGSGTDFTLTI |
| | | | SSLQAEDLADYYCQQYSNYPWTFGGGTKVEIK |
| | LCDR1 (DNA) | 213 | AAGGCAAGTCAGAACGTGGTGAGTGCTGTGGCC |
| | LCDR2 (DNA) | 214 | AGCGCCTCCAACAGGTACACC |
| | LCDR3 (DNA) | 215 | CAACAGTACAGCAATTACCCTTGGACG |
| | VL (DNA) | 216 | GACATCCAGATGACCCAGTCTCCATCCAGCCTGTCT |
| | | | GCATCTGTAGGAGACAGAGTCACCATCACTTGCAAG |
| | | | GCAAGTCAGAACGTGGTGAGTGCTGTGGCCTGGTA |
| | | | TCAGCAGAAACCAGGGCAGGCTCCTAGGAGGCTGA |
| | | | TCTATAGCGCCTCCAACAGGTACACCGGGGTCCCA |
| | | | GACAGGTTCAGCGGCAGTGGATCTGGGACAGATTT |
| | | | CACTCTCACCATCAGCAGCCTGCAGGCCGAAGATCT |
| | | | GGCAGACTATTACTGTCAACAGTACAGCAATTACCC |
| | | | TTGGACGTTCGGCGGCGGGACCAAGGTGGAAATCA |
| | | | AA |
| | LFR1 (protein) | 217 | DIQMTQSPSSLSASVGDRVTITC |
| | LFR2 (protein) | 218 | WYQQKPGQAPRRLIY |
| | LFR3 (protein) | 219 | GVPDRFSGSGSGTDFTLTISSLQAEDLADYYC |
| | LFR4 (protein) | 220 | FGGGTKVEIK |
| | LFR1 (DNA) | 221 | GACATCCAGATGACCCAGTCTCCATCCAGCCTGTCT |
| | | | GCATCTGTAGGAGACAGAGTCACCATCACTTGC |
| | LFR2 (DNA) | 222 | TGGTATCAGCAGAAACCAGGGCAGGCTCCTAGGAG |
| | | | GCTGATCTAT |
| | LFR3 (DNA) | 223 | GGGGTCCCAGACAGGTTCAGCGGCAGTGGATCTGG |
| | | | GACAGATTTCACTCTCACCATCAGCAGCCTGCAGGC |
| | | | CGAAGATCTGGCAGACTATTACTGT |
| | LFR4 (DNA) | 224 | TTCGGCGGCGGGACCAAGGTGGAAATCAAA |
| Girentuximab | IgG1 HC | 225 | ASTKGPSVFPLAPSSKSTSGGTAALGCLVKDYFPEPVT |
| unmodified | (protein) | | VSWNSGALTSGVHTFPAVLQSSGLYSLSSVVTVPSSS |
| human IgG1 | | | LGTQTYICNVNHKPSNTKVDKKVEPKSCDKTHTCPPC |
| constant chain | | | PAPELLGGPSVFLFPPKPKDTLMISRTPEVTCVVVDVS |
| region | | | HEDPEVKFNWYVDGVEVHNAKTKPREEQYNSTYRVV |
| | | | SVLTVLHQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNHYTQKSLSLSPGK |
| Girentuximab | IgG1 H310A | 226 | ASTKGPSVFPLAPSSKSTSGGTAALGCLVKDYFPEPVT |
| modified human | H435Q HC | | VSWNSGALTSGVHTFPAVLQSSGLYSLSSVVTVPSSS |
| IgG1 constant | (protein) | | LGTQTYICNVNHKPSNTKVDKKVEPKSCDKTHTCPPC |
| chain region | | | PAPELLGGPSVFLFPPKPKDTLMISRTPEVTCVVVDVS |
| | | | HEDPEVKFNWYVDGVEVHNAKTKPREEQYNSTYRVV |
| | | | SVLTVLAQDWLNGKEYKCKVSNKALPAPIEKTISKAKG |
| | | | QPREPQVYTLPPSRDELTKNQVSLTCLVKGFYPSDIAV |
| | | | EWESNGQPENNYKTTPPVLDSDGSFFLYSKLTVDKSR |
| | | | WQQGNVFSCSVMHEALHNQYTQKSLSLSPGK |
| Girentuximab | IgG4 S228P | 227 | ASTKGPSVFPLAPCSRSTSESTAALGCLVKDYFPEPVT |
| modified human | L235E HC | | VSWNSGALTSGVHTFPAVLQSSGLYSLSSVVTVPSSS |
| IgG4 constant | (protein) | | LGTKTYTCNVDHKPSNTKVDKRVESKYGPPCPPCPAP |
| chain region | | | EFEGGPSVFLFPPKPKDTLMISRTPEVTCVVVDVSQED |
| | | | PEVQFNWYVDGVEVHNAKTKPREEQFNSTYRVVSVLT |
| | | | VLHQDWLNGKEYKCKVSNKGLPSSIEKTISKAKGQPR |
| | | | EPQVYTLPPSQEEMTKNQVSLTCLVKGFYPSDIAVEW |
| | | | ESNGQPENNYKTTPPVLDSDGSFFLYSRLTVDKSRWQ |
| | | | EGNVFSCSVMHEALHNHYTQKSLSLSLGK |
| Girentuximab | IgG4 S228P | 228 | ASTKGPSVFPLAPCSRSTSESTAALGCLVKDYFPEPVT |
| modified human | L235E H310A | | VSWNSGALTSGVHTFPAVLQSSGLYSLSSVVTVPSSS |
| IgG4 constant | H435Q HC | | LGTKTYTCNVDHKPSNTKVDKRVESKYGPPCPPCPAP |
| chain region | (protein) | | EFEGGPSVFLFPPKPKDTLMISRTPEVTCVVVDVSQED |
| | | | PEVQFNWYVDGVEVHNAKTKPREEQFNSTYRVVSVLT |
| | | | VLAQDWLNGKEYKCKVSNKGLPSSIEKTISKAKGQPRE |
| | | | PQVYTLPPSQEEMTKNQVSLTCLVKGFYPSDIAVEWE |
| | | | SNGQPENNYKTTPPVLDSDGSFFLYSRLTVDKSRWQE |
| | | | GNVFSCSVMHEALHNQYTQKSLSLSLGK |
| Girentuximab | Human κ LC | 229 | RTVAAPSVFIFPPSDEQLKSGTASVVCLLNNFYPREAK |
| kappa light chain | constant region | | VQWKVDNALQSGNSQESVTEQDSKDSTYSLSSTLTLS |
| constant region | | | KADYEKHKVYACEVTHQGLSSPVTKSFNRGEC |
| Girentuximab | IgG1 VH4 heavy | 230 | EVQLVESGGGLVKPGGSLRLSCAASGFTFSNYYMSW |
| native IgG1 | chain | | VRQAPGKGLELVAAINSDGGITYYLDTVKGRFTISRDN |
| allotype Gm | | | AKNTLYLQMNSLRAEDTALYYCARHRSGYFSMDYWG |
| (1,17) VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLHQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNHYTQKSLSLS |
| | | | PGK |
| hGirentuximab | FcRn-null IgG1 | 231 | EVQLVESGGGLVKPGGSLRLSCAASGFTFSNYYMSW |
| IgG1 allotype | allotype H130A | | VRQAPGKGLELVAAINSDGGITYYLDTVKGRFTISRDN |
| Gm (1,17) | H435Q | | AKNTLYLQMNSLRAEDTALYYCARHRSGYFSMDYWG |
| RADmAb VH4 | | | QGTLVTVSSASTKGPSVFPLAPSSKSTSGGTAALGCLV |
| heavy chain | | | KDYFPEPVTVSWNSGALTSGVHTFPAVLQSSGLYSLS |
| | | | SVVTVPSSSLGTQTYICNVNHKPSNTKVDKKVEPKSCD |
| | | | KTHTCPPCPAPELLGGPSVFLFPPKPKDTLMISRTPEV |
| | | | TCVVVDVSHEDPEVKFNWYVDGVEVHNAKTKPREEQ |
| | | | YNSTYRVVSVLTVLAQDWLNGKEYKCKVSNKALPAPIE |
| | | | KTISKAKGQPREPQVYTLPPSRDELTKNQVSLTCLVKG |
| | | | FYPSDIAVEWESNGQPENNYKTTPPVLDSDGSFFLYS |
| | | | KLTVDKSRWQQGNVFSCSVMHEALHNQYTQKSLSLS |
| | | | PGK |
| hGirentuximab | FcRγ-null IgG4 | 232 | EVQLVESGGGLVKPGGSLRLSCAASGFTFSNYYMSW |
| IgG4 RADmAb | S228P L235E | | VRQAPGKGLELVAAINSDGGITYYLDTVKGRFTISRDN |
| VH4 heavy chain | | | AKNTLYLQMNSLRAEDTALYYCARHRSGYFSMDYWG |
| | | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLHQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNHYTQKSLSLSLG |
| | | | K |
| hGirentuximab | FcRn + FcRγ-null | 233 | EVQLVESGGGLVKPGGSLRLSCAASGFTFSNYYMSW |
| IgG4 RADmAb | IgG4 S228P | | VRQAPGKGLELVAAINSDGGITYYLDTVKGRFTISRDN |
| VH4 heavy chain | L235E H310A | | AKNTLYLQMNSLRAEDTALYYCARHRSGYFSMDYWG |
| | H435Q | | QGTLVTVSSASTKGPSVFPLAPCSRSTSESTAALGCLV |
| | | | KDYFPEPVTVSWNSGALTSGVHTFPAVLQSSGLYSLS |
| | | | SVVTVPSSSLGTKTYTCNVDHKPSNTKVDKRVESKYG |
| | | | PPCPPCPAPEFEGGPSVFLFPPKPKDTLMISRTPEVTC |
| | | | VVVDVSQEDPEVQFNWYVDGVEVHNAKTKPREEQFN |
| | | | STYRVVSVLTVLAQDWLNGKEYKCKVSNKGLPSSIEKT |
| | | | ISKAKGQPREPQVYTLPPSQEEMTKNQVSLTCLVKGF |
| | | | YPSDIAVEWESNGQPENNYKTTPPVLDSDGSFFLYSR |
| | | | LTVDKSRWQEGNVFSCSVMHEALHNQYTQKSLSLSL |
| | | | GK |
| hGirentuximab | Vκ Light Chain | 234 | DIQMTQSPSSLSASVGDRVTITCKASQNVVSAVAWYQ |
| Vκ Light Chain | | | QKPGQAPRLLIYSASNRYTGVPDRFSGSGSGTDFTLTI |
| | | | SSLQAEDLADYYCQQYSNYPWTFGGGTKVEIKRTVAA |
| | | | PSVFIFPPSDEQLKSGTASVVCLLNNFYPREAKVQWKV |
| | | | DNALQSGNSQESVTEQDSKDSTYSLSSTLTLSKADYE |
| | | | KHKVYACEVTHQGLSSPVTKSFNRGEC |

TABLE 3
Amino acid sequence of TGFβECD for use in the molecules of
the invention

| | SEQ ID | |
|---|---|---|
| Description | NO: | Amino acid or nucleotide sequence |
| TGFβRI ECD | 246 | LQCFCHLCTKDNFTCVTDGLCFVSVTETTDKVIHNSM |
| | | CIAEIDLIPRDRPFVCAPSSKTGSVTTTYCCNQDHCN |
| | | KIELPTTVKSSPGLGPVEL |
| TGFβRII isoform A ECD | 247 | IPPHVQKSDVEMEAQKDEIICPSCNRTAHPLRHINND |
| | | MIVTDNNGAVKFPQLCKFCDVRFSTCDNQKSCMSN |
| | | CSITSICEKPQEVCVAVWRKNDENITLETVCHDPKLP |
| | | YHDFILEDAASPKCIMKEKKKPGETFFMCSCSSDECN |
| | | DNIIFSEE |
| TGFβRII isoform B ECD | 248 | IPPHVQKSVNNDMIVTDNNGAVKFPQLCKFCDVRFS |
| | | TCDNQKSCMSNCSITSICEKPQEVCVAVWRKNDENI |
| | | TLETVCHDPKLPYHDFILEDAASPKCIMKEKKKPGET |
| | | FFMCSCSSDECNDNIIFSEEYNTSNPDLLLVIFQ |
| TGFβRII isoform B ECD | 249 | IPPHVQKSVNNDMIVTDNNGAVKFPQLCKFCDVRFS |
| | | TCDNQKSCMSNCSITSICEKPQEVCVAVWRKNDENI |
| | | TLETVCHDPKLPYHDFILEDAASPKCIMKEKKKPGET |
| | | FFMCSCSSDECNDNIIFSEEYNTSNPD |
| TGFβRII isoform B ECD (C | 250 | IPPHVQKSVNNDMIVTDNNGAVKFPQLCKFCDVRFS |
| terminal truncation) | | TCDNQKSCMSNCSITSICEKPQEVCVAVWRKNDENI |
| | | TLETVCHDPKLPYHDFILEDAASPKCIMKEKKKPGET |
| | | FFMCSCSSDECNDNIIFSEE |
| TGFβRII isoform B ECD (C | 251 | TDNNGAVKFPQLCKFCDVRFSTCDNQKSCMSNCSIT |
| terminal truncation and which | | SICEKPQEVCVAVWRKNDENITLETVCHDPKLPYHDF |
| has an N-terminal deletion of | | ILEDAASPKCIMKEKKKPGETFFMCSCSSDECNDNIIF |
| aas 1-14 (Δ14). | | SEE |
| TGFβRII isoform B ECD (C | 252 | QLCKFCDVRFSTCDNQKSCMSNCSITSICEKPQEVC |
| terminal truncation and which | | VAVWRKNDENITLETVCHDPKLPYHDFILEDAASPKCI |
| has an N-terminal deletion of | | MKEKKKPGETFFMCSCSSDECNDNIIFSEE |
| aas 1-25 (Δ25). | | |
| TGFβRIII A isoform | 253 | MTSHYVIAIFALMSSCLATAGPEPGALCELSPVSASH |
| | | PVQALMESFTVLSGCASRGTTGLPQEVHVLNLRTAG |
| | | QGPGQLQREVTLHLNPISSVHIHHKSVVFLLNSPHPL |
| | | VWHLKTERLATGVSRLFLVSEGSVVQFSSANFSLTA |
| | | ETEERNFPHGNEHLLNWARKEYGAVTSFTELKIARNI |
| | | YIKVGEDQVFPPKCNIGKNFLSLNYLAEYLQPKAAEG |
| | | CVMSSQPQNEEVHIIELITPNSNPYSAFQVDITIDIRPS |
| | | QEDLEVVKNLILILKCKKSVNWVIKSFDVKGSLKIIAPN |
| | | SIGFGKESERSMTMTKSIRDDIPSTQGNLVKWALDN |
| | | GYSPITSYTMAPVANRFHLRLENNAEEMGDEEVHTIP |
| | | PELRILLDPGALPALQNPPIRGGEGQNGGLPFPFPDI |
| | | SRRVWNEEGEDGLPRPKDPVIPSIQLFPGLREPEEV |
| | | QGSVDIALSVKCDNEKMIVAVEKDSFQASGYSGMDV |
| | | TLLDPTCKAKMNGTHFVLESPLNGCGTRPRWSALDG |
| | | VVYYNSIVIQVPALGDSSGWPDGYEDLESGDNGFPG |
| | | DMDEGDASLFTRPEIVVFNCSLQQVRNPSSFQEQPH |
| | | GNITFNMELYNTDLFLVPSQGVFSVPENGHVYVEVS |
| | | VTKAEQELGFAIQTCFISPYSNPDRMSHYTIIENICPK |
| | | DESVKFYSPKRVHFPIPQADMDKKRFSFVFKPVENTS |
| | | LLFLQCELTLCTKMEKHPQKLPKCVPPDEACTSLDAS |
| | | IIWAMMQNKKTFTKPLAVIHHEAESKEKGPSMKEPNP |
| | | ISPPIFHGLDTLTVMGIAFAAFVIGALLTGALWYIYSHT |
| | | GETAGRQQVPTSPPASENSSAAHSIGSTQSTPCSSS |
| | | STA |
| TGFβRIII B isoform | 254 | MTSHYVIAIFALMSSCLATAGPEPGALCELSPVSASH |
| | | PVQALMESFTVLSGCASRGTTGLPQEVHVLNLRTAG |
| | | QGPGQLQREVTLHLNPISSVHIHHKSVVFLLNSPHPL |
| | | VWHLKTERLATGVSRLFLVSEGSVVQFSSANFSLTA |
| | | ETEERNFPHGNEHLLNWARKEYGAVTSFTELKIARNI |
| | | YIKVGEDQVFPPKCNIGKNFLSLNYLAEYLQPKAAEG |
| | | CVMSSQPQNEEVHIIELITPNSNPYSAFQVDITIDIRPS |
| | | QEDLEVVKNLILILKCKKSVNWVIKSFDVKGSLKIIAPN |
| | | SIGFGKESERSMTMTKSIRDDIPSTQGNLVKWALDN |
| | | GYSPITSYTMAPVANRFHLRLENNEEMGDEEVHTIPP |
| | | ELRILLDPGALPALQNPPIRGGEGQNGGLPFPFPDIS |
| | | RRVWNEEGEDGLPRPKDPVIPSIQLFPGLREPEEVQ |
| | | GSVDIALSVKCDNEKMIVAVEKDSFQASGYSGMDVT |
| | | LLDPTCKAKMNGTHFVLESPLNGCGTRPRWSALDG |
| | | VVYYNSIVIQVPALGDSSGWPDGYEDLESGDNGFPG |
| | | DMDEGDASLFTRPEIVVFNCSLQQVRNPSSFQEQPH |
| | | GNITFNMELYNTDLFLVPSQGVFSVPENGHVYVEVS |
| | | VTKAEQELGFAIQTCFISPYSNPDRMSHYTIIENICPK |
| | | DESVKFYSPKRVHFPIPQADMDKKRFSFVFKPVENTS |
| | | LLFLQCELTLCTKMEKHPQKLPKCVPPDEACTSLDAS |
| | | IIWAMMQNKKTFTKPLAVIHHEAESKEKGPSMKEPNP |
| | | ISPPIFHGLDTLTVMGIAFAAFVIGALLTGALWYIYSHT |
| | | GETAGRQQVPTSPPASENSSAAHSIGSTQSTPCSSS |
| | | STA |
| TGFβRII isoform B ECD | 321 | TIPPHVQKSVNNDMIVTDNNGAVKFPQLCKFCDVRF |
| | | STCDNQKSCMSNCSITSICEKPQEVCVAVWRKNDEN |
| | | ITLETVCHDPKLPYHDFILEDAASPKCIMKEKKKPGET |
| | | FFMCSCSSDECNDNIIFSEEYNTSNPD |

TABLE 4
Exemplary multifunctional antibodies of the invention

| | SEQ ID | |
|---|---|---|
| Description | NO: | Amino acid or nucleotide sequence |
| Anti-CAIX + TGFβ ecd | 255 | MGWSCIILFLVATATGVHSDVKLVESGGGLVKLGGSL |
| Heavy chain sequence | | KLSCAASGFTFSNYYMSWVRQTPEKRLELVAAINSD |
| (linker region between antibody | | GGITYYLDTVKGRFTISRDNAKNTLYLQMSSLKSEDT |
| heavy chain and TGFβ ecd | | ALFYCARHRSGYFSMDYWGQGTSVTVSSASTKGPS |
| underlined; N terminal signal peptide, | | VFPLAPSSKSTSGGTAALGCLVKDYFPEPVTVSWN |
| cleavable, italicised; variable region | | SGALTSGVHTFPAVLQSSGLYSLSSVVTVPSSSLGT |
| normal text; constant region in bold) | | QTYICNVNHKPSNTKVDKKVEPKSCDKTHTCPPCPA |
| | | PELLGGPSVFLFPPKPKDTLMISRTPEVTCVVVDVSH |
| | | EDPEVKFNWYVDGVEVHNAKTKPREEQYNSTYRVV |
| | | SVLTVLHQDWLNGKEYKCKVSNKALPAPIEKTISKA |
| | | KGQPREPQVYTLPPSRDELTKNQVSLTCLVKGFYPS |
| | | DIAVEWESNGQPENNYKTTPPVLDSDGSFFLYSKLT |
| | | VDKSRWQQGNVFSCSVMHEALHNHYTQKSLSLSP |
| | | G[K/A]GGGGSGGGGSGGGGSTIPPHVQKSVNNDMI |
| | | VTDNNGAVKFPQLCKFCDVRFSTCDNQKSCMSNCSI |
| | | TSICEKPQEVCVAVWRKNDENITLETVCHDPKLPYHD |
| | | FILEDAASPKCIMKEKKKPGETFFMCSCSSDECNDNII |
| | | FSEEYNTSNPD |
| Anti-CAIX | 256 | MGWSCIILFLVATATGVHSDIVMTQSQRFMSTTVGDR |
| Light chain sequence (N terminal | | VSITCKASQNVVSAVAWYQQKPGQSPKLLIYSASNR |
| signal peptide, cleavable, italicised; | | YTGVPDRFTGSGSGTDFTLTISNMQSEDLADFFCQQ |
| variable region normal text; constant | | YSNYPWTFGGGTKLEIKRTVAAPSVFIFPPSDEQLKS |
| region in bold) | | GTASVVCLLNNFYPREAKVQWKVDNALQSGNSQES |
| | | VTEQDSKDSTYSLSSTLTLSKADYEKHKVYACEVTH |
| | | QGLSSPVTKSFNRGEC |
| Anti-CAIX + TGFβ ecd | 257 | DVKLVESGGGLVKLGGSLKLSCAASGFTFSNYYMSW |
| Heavy chain sequence | | VRQTPEKRLELVAAINSDGGITYYLDTVKGRFTISRDN |
| Mature sequence; variable region | | AKNTLYLQMSSLKSEDTALFYCARHRSGYFSMDYW |
| normal text; constant region in bold | | GQGTSVTVSSASTKGPSVFPLAPSSKSTSGGTAAL |
| | | GCLVKDYFPEPVTVSWNSGALTSGVHTFPAVLQSS |
| | | GLYSLSSVVTVPSSSLGTQTYICNVNHKPSNTKVDK |
| | | KVEPKSCDKTHTCPPCPAPELLGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALPAPIEKTISKAKGQPREPQVYTLPPSRD |
| | | ELTKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGKGGGGSGGGGSGGGG |
| | | STIPPHVQKSVNNDMIVTDNNGAVKFPQLCKFCDVR |
| | | FSTCDNQKSCMSNCSITSICEKPQEVCVAVWRKNDE |
| | | NITLETVCHDPKLPYHDFILEDAASPKCIMKEKKKPGE |
| | | TFFMCSCSSDECNDNIIFSEEYNTSNPD |
| Anti-CAIX + TGFβ ecd | 258 | DVKLVESGGGLVKLGGSLKLSCAASGFTFSNYYMSW |
| Heavy chain sequence | | VRQTPEKRLELVAAINSDGGITYYLDTVKGRFTISRDN |
| Mature sequence; variable region | | AKNTLYLQMSSLKSEDTALFYCARHRSGYFSMDYW |
| normal text; constant region in bold; | | GQGTSVTVSSASTKGPSVFPLAPSSKSTSGGTAAL |
| linker underlined; | | GCLVKDYFPEPVTVSWNSGALTSGVHTFPAVLQSS |
| C terminal K to A substitution in heavy | | GLYSLSSVVTVPSSSLGTQTYICNVNHKPSNTKVDK |
| chain | | KVEPKSCDKTHTCPPCPAPELLGGPSVFLFPPKPKD |
| | | TLMISRTPEVTCVVVDVSHEDPEVKFNWYVDGVEVH |
| | | NAKTKPREEQYNSTYRVVSVLTVLHQDWLNGKEYK |
| | | CKVSNKALPAPIEKTISKAKGQPREPQVYTLPPSRD |
| | | ELTKNQVSLTCLVKGFYPSDIAVEWESNGQPENNYK |
| | | TTPPVLDSDGSFFLYSKLTVDKSRWQQGNVFSCSV |
| | | MHEALHNHYTQKSLSLSPGAGGGGSGGGGSGGGG |
| | | STIPPHVQKSVNNDMIVTDNNGAVKFPQLCKFCDVR |
| | | FSTCDNQKSCMSNCSITSICEKPQEVCVAVWRKNDE |
| | | NITLETVCHDPKLPYHDFILEDAASPKCIMKEKKKPGE |
| | | TFFMCSCSSDECNDNIIFSEEYNTSNPD |
| Anti-CAIX | 259 | DIVMTQSQRFMSTTVGDRVSITCKASQNVVSAVAWY |
| Light chain sequence-mature | | QQKPGQSPKLLIYSASNRYTGVPDRFTGSGSGTDFT |
| sequence; variable region normal text; | | LTISNMQSEDLADFFCQQYSNYPWTFGGGTKLEIKR |
| constant region in bold | | TVAAPSVFIFPPSDEQLKSGTASVVCLLNNFYPREA |
| | | KVQWKVDNALQSGNSQESVTEQDSKDSTYSLSSTL |
| | | TLSKADYEKHKVYACEVTHQGLSSPVTKSFNRGEC |
| Anti-PSMA + TGFβ ecd | 260 | MGWSCIILFLVATATGVHSEVQLVQSGPEVKKPGATV |
| Heavy chain sequence | | KISCKTSGYTFTEYTIHWVKQAPGKGLEWIGNINPNN |
| (linker region between antibody | | GGTTYNQKFEDKATLTVDKSTDTAYMELSSLRSEDT |
| heavy chain and TGFβ ecd | | AVYYCAAGWNFDYWGQGTLLTVSSASTKGPSVFPL |
| underlined; N terminal signal peptide, | | APSSKSTSGGTAALGCLVKDYFPEPVTVSWNSGAL |
| cleavable, italicised; variable region | | TSGVHTFPAVLQSSGLYSLSSVVTVPSSSLGTQTYIC |
| normal text; constant region in bold) | | NVNHKPSNTKVDKKVEPKSCDKTHTCPPCPAPELL |
| | | GGPSVFLFPPKPKDTLMISRTPEVTCVVVDVSHEDP |
| | | EVKFNWYVDGVEVHNAKTKPREEQYNSTYRVVSVL |
| | | TVLHQDWLNGKEYKCKVSNKALPAPIEKTISKAKGQ |
| | | PREPQVYTLPPSRDELTKNQVSLTCLVKGFYPSDIA |
| | | VEWESNGQPENNYKTTPPVLDSDGSFFLYSKLTVD |
| | | KSRWQQGNVFSCSVMHEALHNHYTQKSLSLSPG[K/A] |
| | | GGGGSGGGGSGGGGSTIPPHVQKSVNNDMIVTD |
| | | NNGAVKFPQLCKFCDVRFSTCDNQKSCMSNCSITSI |
| | | CEKPQEVCVAVWRKNDENITLETVCHDPKLPYHDFIL |
| | | EDAASPKCIMKEKKKPGETFFMCSCSSDECNDNIIFS |
| | | EEYNTSNPD |
| Anti-PSMA | 261 | MGWSCIILFLVATATGVHSDIQMTQSPSSLSTSVGDR |
| Light chain sequence (N terminal | | VTLTCKASQDVGTAVDWYQQKPGPSPKLLIYWASTR |
| signal peptide, cleavable, italicised; | | HTGIPSRFSGSGSGTDFTLTISSLQPEDFADYYCQQY |
| variable region normal text; constant | | NSYPLTFGPGTKVDIKRTVAAPSVFIFPPSDEQLKSG |
| region in bold) | | TASVVCLLNNFYPREAKVQWKVDNALQSGNSQESV |
| | | TEQDSKDSTYSLSSTLTLSKADYEKHKVYACEVTHQ |
| | | GLSSPVTKSFNRGEC |
| Anti-PSMA + TGFβ ecd | 262 | EVQLVQSGPEVKKPGATVKISCKTSGYTFTEYTIHWV |
| Heavy chain sequence | | KQAPGKGLEWIGNINPNNGGTTYNQKFEDKATLTVD |
| Mature sequence; variable region | | KSTDTAYMELSSLRSEDTAVYYCAAGWNFDYWGQG |
| normal text; constant region in bold) | | TLLTVSSASTKGPSVFPLAPSSKSTSGGTAALGCLV |
| | | KDYFPEPVTVSWNSGALTSGVHTFPAVLQSSGLYSL |
| | | SSVVTVPSSSLGTQTYICNVNHKPSNTKVDKKVEPK |
| | | SCDKTHTCPPCPAPELLGGPSVFLFPPKPKDTLMIS |
| | | RTPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGKGGGGSGGGGSGGGGSTIPP |
| | | HVQKSVNNDMIVTDNNGAVKFPQLCKFCDVRFSTCD |
| | | NQKSCMSNCSITSICEKPQEVCVAVWRKNDENITLET |
| | | VCHDPKLPYHDFILEDAASPKCIMKEKKKPGETFFMC |
| | | SCSSDECNDNIIFSEEYNTSNPD |
| Anti-PSMA + TGFβ ecd | 263 | EVQLVQSGPEVKKPGATVKISCKTSGYTFTEYTIHWV |
| Heavy chain sequence | | KQAPGKGLEWIGNINPNNGGTTYNQKFEDKATLTVD |
| Mature sequence; variable region | | KSTDTAYMELSSLRSEDTAVYYCAAGWNFDYWGQG |
| normal text; constant region in bold) | | TLLTVSSASTKGPSVFPLAPSSKSTSGGTAALGCLV |
| C terminal K to A substitution in | | KDYFPEPVTVSWNSGALTSGVHTFPAVLQSSGLYSL |
| heavy chain. | | SSVVTVPSSSLGTQTYICNVNHKPSNTKVDKKVEPK |
| | | SCDKTHTCPPCPAPELLGGPSVFLFPPKPKDTLMIS |
| | | RTPEVTCVVVDVSHEDPEVKFNWYVDGVEVHNAKT |
| | | KPREEQYNSTYRVVSVLTVLHQDWLNGKEYKCKVS |
| | | NKALPAPIEKTISKAKGQPREPQVYTLPPSRDELTK |
| | | NQVSLTCLVKGFYPSDIAVEWESNGQPENNYKTTPP |
| | | VLDSDGSFFLYSKLTVDKSRWQQGNVFSCSVMHEA |
| | | LHNHYTQKSLSLSPGAGGGGSGGGGSGGGGSTIPP |
| | | HVQKSVNNDMIVTDNNGAVKFPQLCKFCDVRFSTCD |
| | | NQKSCMSNCSITSICEKPQEVCVAVWRKNDENITLET |
| | | VCHDPKLPYHDFILEDAASPKCIMKEKKKPGETFFMC |
| | | SCSSDECNDNIIFSEEYNTSNPD |
| Anti-PSMA | 264 | DIQMTQSPSSLSTSVGDRVTLTCKASQDVGTAVDWY |
| Light chain sequence-mature | | QQKPGPSPKLLIYWASTRHTGIPSRFSGSGSGTDFTL |
| sequence; variable region normal text; | | TISSLQPEDFADYYCQQYNSYPLTFGPGTKVDIKRTV |
| constant region in bold) | | AAPSVFIFPPSDEQLKSGTASVVCLLNNFYPREAKV |
| | | QWKVDNALQSGNSQESVTEQDSKDSTYSLSSTLTL |
| | | SKADYEKHKVYACEVTHQGLSSPVTKSFNRGEC |

TABLE 5
Summary of amino acid of PDGFRa-binding antibodies or
antigen binding fragments thereof for use in the invention

| Antibody ID | Region | SEQ ID NO: | Amino acid sequence |
|---|---|---|---|
| olaratumab | Heavy chain | 265 | QLQLQESGPGLVKPSETLSLTCTVSGGSINSSSYY |
| | variable | | WGWLRQSPGKGLEWIGSFFYTGSTYYNPSLRSRL |
| | | | TISVDTSKNQFSLMLSSVTAADTAVYYCARQSTYYY |
| | | | GSGNYYGWFDRWDQGTLVTVSS |
| | Light chain | 266 | EIVLTQSPATLSLSPGERATLSCRASQSVSSYLAWY |
| | variable | | QQKPGQAPRLLIYDASNRATGIPARFSGSGSGTDF |
| | | | TLTISSLEPEDFAVYYCQQRSNWPPAFGQGTKVEIK |
| | HCDR1 | 267 | GGSINGSSSYY |
| | (Chothia) | | |
| | HCDR2 | 268 | FFYTGST |
| | (Chothia) | | |
| | HCDR3 | 269 | ARQSTYYYGSGNYYGWFDR |
| | (Chothia) | | |
| | LCDR1 | 270 | QSVSSY |
| | (Chothia) | | |
| | LCDR2 | 271 | DAS |
| | (Chothia) | | |
| | LCDR3 | 272 | QQRSNWPPA |
| | (Chothia) | | |
| | HFR1 | 273 | QLQLQESGPGLVKPSETLSLTCTVS |
| | (Chothia) | | |
| | HFR2 | 274 | WGWLRQSPGKGLEWIGS |
| | (Chothia) | | |
| | HFR3 | 275 | YYNPSLRSRLTISVDTSKNQFSLMLSSVTAADTAVY |
| | (Chothia) | | Y |
| | HFR4 | 276 | WDQGTLVTVSS |
| | (Chothia) | | |
| | LFR1 | 277 | EIVLTQSPATLSLSPGERATLSCRAS |
| | (Chothia) | | |
| | LFR2 | 278 | LAWYQQKPGQAPRLLIY |
| | (Chothia) | | |
| | LFR3 | 279 | NRATGIPARFSGSGSGTDFTLTISSLEPEDFAVYY |
| | (Chothia) | | |
| | LFR4 | 300 | FGQGTKVEIK |
| | (Chothia) | | |
| | Heavy chain | 301 | ASTKGPSVFPLAPSSKSTSGGTAALGCLVKDYFPE |
| | constant | | PVTVSWNSGALTSGVHTFPAVLQSSGLYSLSSVVT |
| | | | VPSSSLGTQTYICNVNHKPSNTKVDKRVEPKSCDK |
| | | | THTCPPCPAPELLGGPSVFLFPPKPKDTLMISRTPE |
| | | | VTCVVVDVSHEDPEVKFNWYVDGVEVHNAKTKPR |
| | | | EEQYNSTYRVVSVLTVLHQDWLNGKEYKCKVSNK |
| | | | ALPAPIEKTISKAKGQPREPQVYTLPPSREEMTKNQ |
| | | | VSLTCLVKGFYPSDIAVEWESNGQPENNYKTTPPV |
| | | | LDSDGSFFLYSKLTVDKSRWQQGNVFSCSVMHEA |
| | | | LHNHYTQKSLSLSPGK |
| | Light chain | 302 | RTVAAPSVFIFPPSDEQLKSGTASVVCLLNNFYPRE |
| | constant | | AKVQWKVDNALQSGNSQESVTEQDSKDSTYSLSS |
| | | | TLTLSKADYEKHKVYACEVTHQGLSSPVTKSFNRG |
| | | | EC |

TABLE 6
Summary of amino acid of La/SSB-binding antibodies or antigen
binding fragments thereof for use in the invention

| | | SEQ ID | |
|---|---|---|---|
| Antibody ID | Region | NO: | Amino acid or nucleotide sequence |
| apoMab | Heavy chain | 303 | EVQLQQPGAELVKPGASVKLSCKASGYAFTHYYIYWI |
| | variable | | KQRPGQGLEWIGGVNPSNGGTHFNEKFKSKATLTV |
| | | | DKSSSTAYMQLSSLTSEDSAVYYCTRSEYDYGLGFA |
| | | | YWGQGTLLTVSS |
| | Light chain | 304 | EIVMSQSPSSLAVSAGEKVTMSCKSSQSLLNSRTPK |
| | variable | | NYLAWYQQKPGQSPKLLIYWASTRKSGVPDRFTGS |
| | | | GSGTDFTLTISSVQAEDLAVYYCKQSYNLLTFGAGTK |
| | | | LELK |
| | HCDR1 | 305 | HYYIY |
| | (Kabat) | | |
| | HCDR2 | 306 | GVNPSNGGTHFNEKFKS |
| | (Kabat) | | |
| | HCDR3 | 307 | SEYDYGLGFAY |
| | (Kabat) | | |
| | LCDR1 | 308 | KSSQSLLNSRTPKNYLA |
| | (Kabat) | | |
| | LCDR2 | 309 | WASTRKS |
| | (Kabat) | | |
| | LCDR3 | 310 | KQSYNLLT |
| | (Kabat) | | |
| | HFR1 | 311 | EVQLQQPGAELVKPGASVKLSCKASGYAFT |
| | (Kabat) | | |
| | HFR2 | 312 | WIKQRPGQGLEWIG |
| | (Kabat) | | |
| | HFR3 | 313 | KATLTVDKSSSTAYMQLSSLTSEDSAVYYCTR |
| | (Kabat) | | |
| | HDR4 | 314 | WGQGTLLTVSS |
| | (Kabat) | | |
| | LFR1 | 315 | EIVMSQSPSSLAVSAGEKVTMSC |
| | (Kabat) | | |
| | LFR2 | 316 | WYQQKPGQSPKLLIY |
| | (Kabat) | | |
| | LFR3 | 317 | GVPDRFTGSGSGTDFTLTISSVQAEDLAVYYC |
| | (Kabat) | | |
| | LFR4 | 318 | GAGTKLELK |
| | (Kabat) | | |
| | Heavy chain | 319 | ASTKGPSVFPLAPSSKSTSGGTAALGCLVKDYFPEP |
| | constant | | VTVSWNSGALTSGVHTFPAVLQSSGLYSLSSVVTVP |
| | [optional K | | SSSLGTQTYICNVNHKPSNTKVDKKVEPKSCDKTHT |
| | to A | | CPPCPAPELLGGPSVFLFPPKPKDTLMISRTPEVTCV |
| | substitution at | | VVDVSHEDPEVKFNWYVDGVEVHNAKTKPREEQYN |
| | position 322] | | STYRVVSVLTVLHQDWLNGKEYKC[K/A]VSNKALPAP |
| | | | IEKTISKAKGQPREPQVYTLPPSREEMTKNQVSLTCL |
| | | | VKGFYPSDIAVEWESNGQPENNYKTTPPVLDSDGSF |
| | | | FLYSKLTVDKSRWQQGNVFSCSVMHEALHNHYTQK |
| | | | SLSLSPGK |
| | Light chain | 320 | RTVAAPSVFIFPPSDEQLKSGTASVVCLLNNFYPREA |
| | constant | | KVQWKVDNALQSGNSQESVTEQDSKDSTYSLSSTLT |
| | | | LSKADYEKHKVYACEVTHQGLSSPVTKSFNRGEC |

### EXAMPLES

### Example 1: Anti-CAIX-TGFβ Trap and External Beam Radiation (EBRT) to Enhance Efficacy of Checkpoint Inhibitors

A molecule comprising an antigen binding domain for binding to CAIX and an ECD derived from TGFβR II is obtained by expressing in a mammalian cell line, a first expression construct (encoding a fusion protein comprising the sequence of SEQ ID NO: 255) and a second expression construct (encoding an antibody light chain as set forth in SEQ ID NO: 256). Upon expression of the proteins, the protein encoded by the first construct and the protein encoded by the second construct associate to form a single molecule through intramolecular disulphide bonding. This molecule is termed an anti-CAIX-TGFβ TRAP.

Standard in vitro assays of the molecule are conducted to confirm binding to CAIX (including CAIX-expressing tumour cells) and to TGFβ.

A mouse model is established in which tumours expressing human CAIX are engrafted into mice. Mice are split into 5 groups as follows:
1) control: no treatment;
2) anti-PD-1;
3) treatment with EBRT;
4) treatment with EBRT+anti PD-1;
5) treatment with EBRT+anti PD-1+anti-CAIX TGFβ-trap.

Mice are monitored for tumour growth. Some mice are sacrificed at fixed time points to monitor T cell infiltration and fibrosis.

The extent of fibrosis and T cell infiltration at the tumour site is assessed between treatment groups. The extent of radiation-induced fibrosis in treatment group 5 will be lower compared to groups 3 and 4. The extent of T cell infiltration in group 5 will be higher than T cell infiltration in all other groups.

### Example 2: Anti-CAIX-TGFβ TRAP and Molecular Targeted Radiation to Enhance Checkpoint Inhibitor Treatment

A molecule comprising an antigen binding domain for binding to CAIX and an ECD derived from TGFβR II is obtained according to the method of Example 1. In this example, the molecule is further conjugated to the radionuclide 177-Lutetium.

Standard in vitro assays of the molecule are conducted to confirm binding of the molecule to CAIX (including CAIX-expressing tumour cells) and to TGFβ.

A mouse model is established in which tumours expressing human CAIX are engrafted into mice. Mice are split into 8 groups as follows:
1) control: no treatment;
2) anti-PD-1;
3) treatment with ¹⁷⁷-Lu labelled anti-CAIX binding protein;
4) treatment with ¹⁷⁷-Lu labelled anti-CAIX-TGFβ-trap;
5) treatment with ¹⁷⁷-Lu labelled anti-CAIX binding protein+anti PD-1;
6) treatment with ¹⁷⁷-Lu labelled anti-CAIX-TGFβ-trap+anti PD-1;
7) treatment with ¹⁷⁷-Lu labelled anti-CAIX binding protein followed by anti PD-1+anti-CAIX-TGFβ-trap;
8) treatment with ¹⁷⁷-Lu labelled anti-CAIX TGFβ-trap followed by anti PD-1+anti-CAIX-TGFβ-trap.

Mice are monitored for tumour growth. Some mice are sacrificed at fixed time points to monitor T cell infiltration and fibrosis.

The extent of fibrosis and T cell infiltration at the tumour site is assessed between treatment groups. The extent of radiation-induced fibrosis in treatment groups 4,6,7,8 will be lower compared to groups 3 and 5. The extent of T cell infiltration in groups that have been irradiated and treated with a TGFβ trap will be higher than in groups that have been irradiated but not treated with a TGFβ trap.

### Example 3: Characterisation of Anti-CAIX-TGFβ TRAP (TLX250 Trap)

A molecule comprising an antigen binding domain for binding to CAIX and an ECD derived from TGFβR II was obtained according to the method of Example 1 (referred to as TLX250 Trap).

Analysis of dimerisation and purity of resultant TLX250 trap containing samples is shown in FIG. 1.

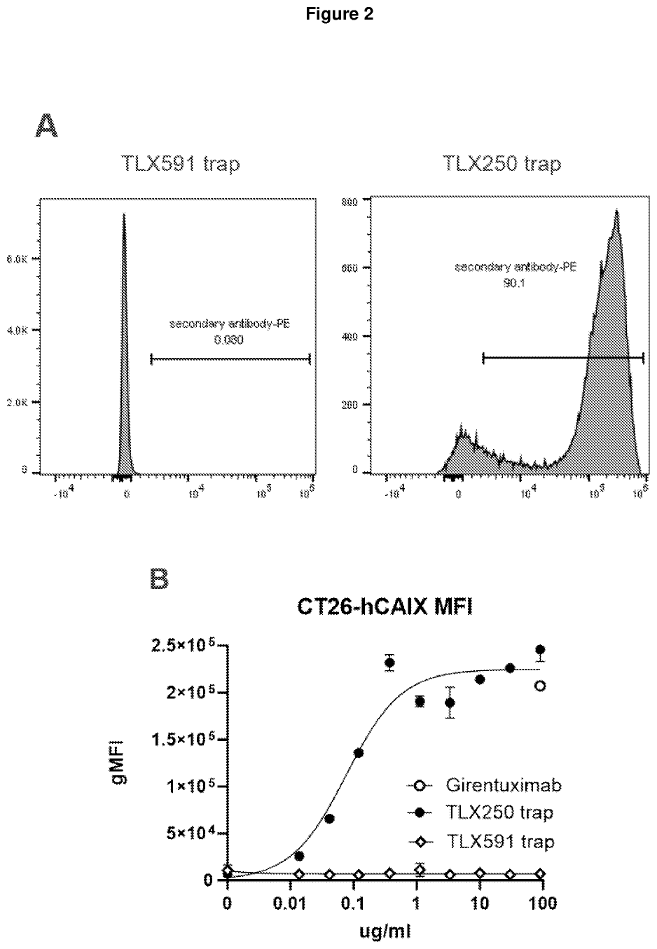

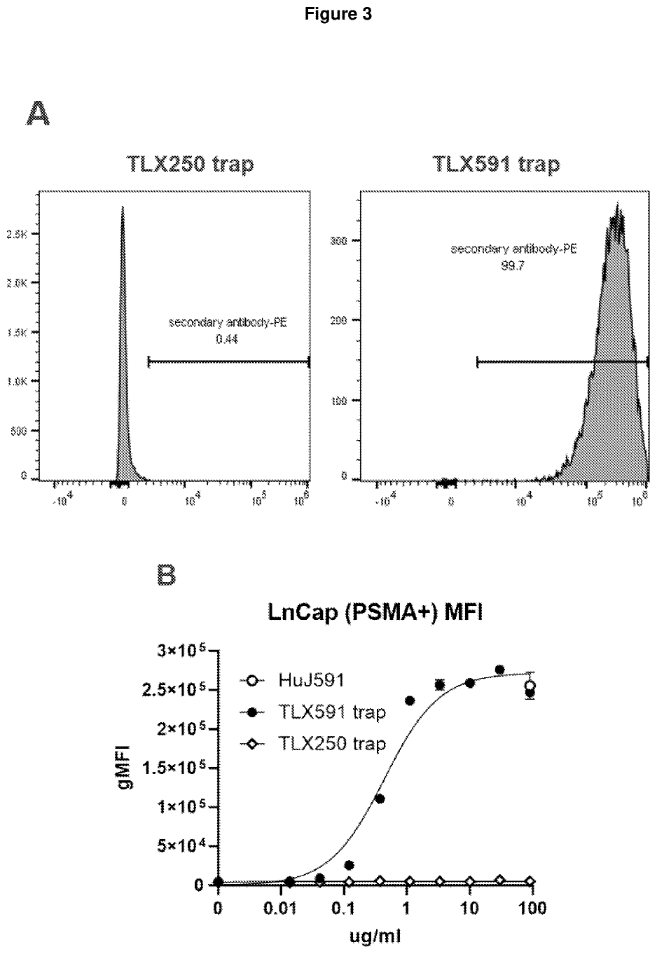

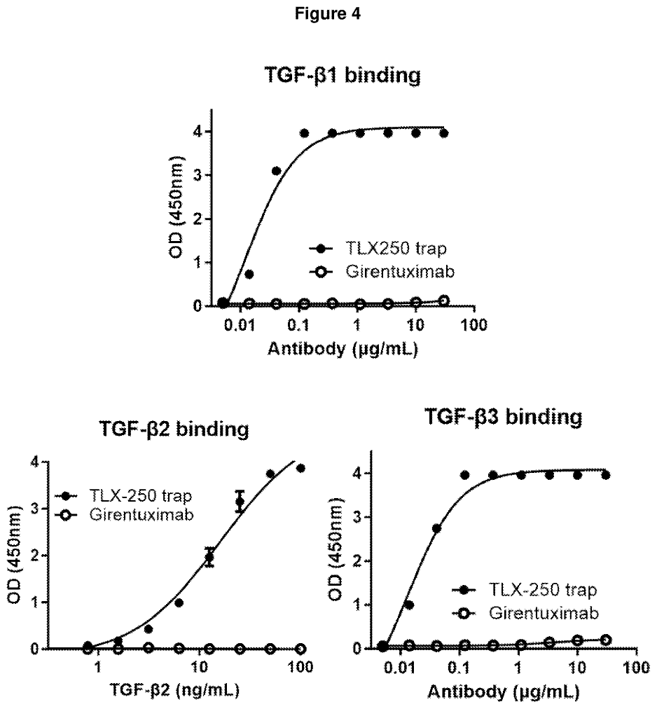

In vitro, TLX250 trap was shown to specifically bind to CT26-hCAIX cells in a dose dependent manner (See FIGS. 2 and 3). TLX250 trap was also shown to specifically bind to TGFβ1, TGFβ2 and TGFβ3 (FIG. 4).

### Example 4: Anti-PSMA-TGFβ Trap and External Beam Radiation (EBRT) to Enhance Efficacy of Checkpoint Inhibitors

A molecule comprising an antigen binding domain for binding to PSMA and an ECD derived from TGFβR II is obtained by expressing in a mammalian cell line, a first expression construct (encoding a fusion protein comprising the sequence of SEQ ID NO: 260) and a second expression construct (encoding an antibody light chain as set forth in SEQ ID NO: 261). Upon expression of the proteins, the protein encoded by the first construct and the protein encoded by the second construct associate to form a single molecule through intramolecular disulphide bonding. This molecule is termed an anti-PSMA-TGFβ TRAP.

Standard in vitro assays of the molecule are conducted to confirm binding to PSMA (including PSMA-expressing tumour cells) and to TGFβ.

A mouse model is established in which tumours expressing human PSMA are engrafted into mice. Mice are split into 5 groups as follows:
1) control: no treatment;
2) anti-PD-1;
3) treatment with EBRT;
4) treatment with EBRT+anti PD-1;
5) treatment with EBRT+anti PD-1+anti-PSMA TGFβ-trap.

Mice are monitored for tumour growth. Some mice are sacrificed at fixed time points to monitor T cell infiltration and fibrosis.

The extent of fibrosis and T cell infiltration at the tumour site is assessed between treatment groups. The extent of radiation-induced fibrosis in treatment group 5 will be lower compared to groups 3 and 4. The extent of T cell infiltration in group 5 will be higher than T cell infiltration in all other groups.

### Example 5: Anti-PSMA-TGFβ TRAP and Molecular Targeted Radiation to Enhance Checkpoint Inhibitor Treatment

A molecule comprising an antigen binding domain for binding to PSMA and an ECD derived from TGFβR II is obtained according to the method of Example 1. In this example, the molecule is further conjugated to the radionuclide 177-Lutetium.

Standard in vitro assays of the molecule are conducted to confirm binding of the molecule to PSMA (including PSMA-expressing tumour cells) and to TGFβ.

A mouse model is established in which tumours expressing human PSMA are engrafted into mice. Mice are split into 8 groups as follows:
1) control: no treatment;
2) anti-PD-1;
3) treatment with ¹⁷⁷-Lu labelled anti-PSMA binding protein;
4) treatment with ¹⁷⁷-Lu labelled anti-PSMA-TGFβ-trap;
5) treatment with ¹⁷⁷-Lu labelled anti-PSMA binding protein+anti PD-1;
6) treatment with ¹⁷⁷-Lu labelled anti-PSMA-TGFβ-trap+anti PD-1;
7) treatment with ¹⁷⁷-Lu labelled anti-PSMA binding protein followed by anti PD-1+anti-PSMA-TGFβ-trap;
8) treatment with ¹⁷⁷-Lu labelled anti-PSMA TGFβ-trap followed by anti PD-1+anti-PSMA-TGFβ-trap.

Mice are monitored for tumour growth. Some mice are sacrificed at fixed time points to monitor T cell infiltration and fibrosis.

The extent of fibrosis and T cell infiltration at the tumour site is assessed between treatment groups. The extent of radiation-induced fibrosis in treatment groups 4,6,7,8 will be lower compared to groups 3 and 5. The extent of T cell infiltration in groups that have been irradiated and treated with a TGFβ trap will be higher than in groups that have been irradiated but not treated with a TGFβ trap.

### Example 6: Characterisation of Anti-PSMA-TGFβ TRAP (TLX591 Trap)

A molecule comprising an antigen binding domain for binding to CAIX and an ECD derived from TGFβR II was obtained according to the method of Example 4 (referred to as TLX591 Trap).

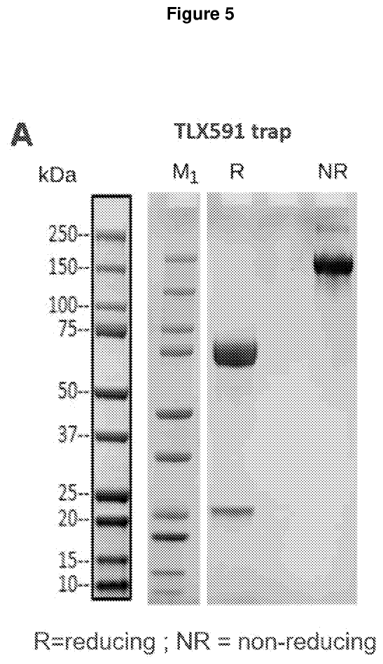

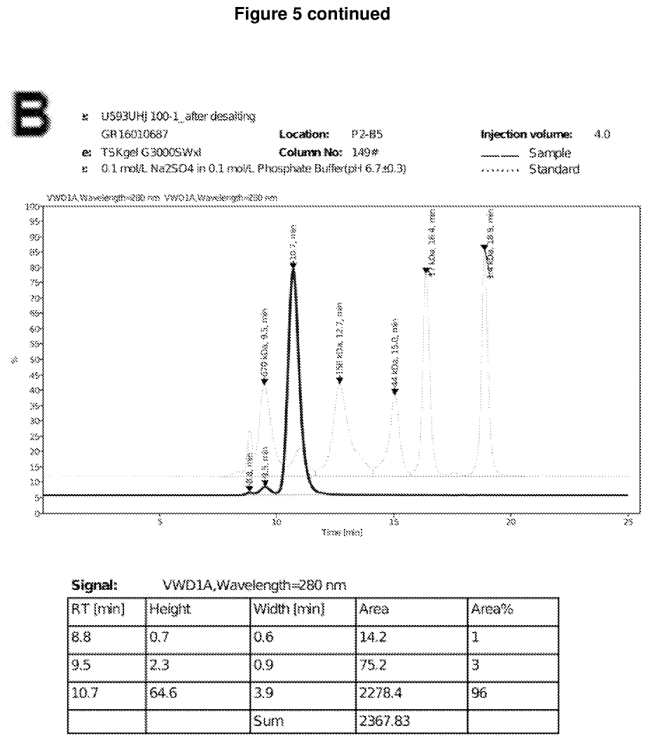

Analysis of dimerisation and purity of the resultant TLX591 trap containing samples is shown in FIG. 5.

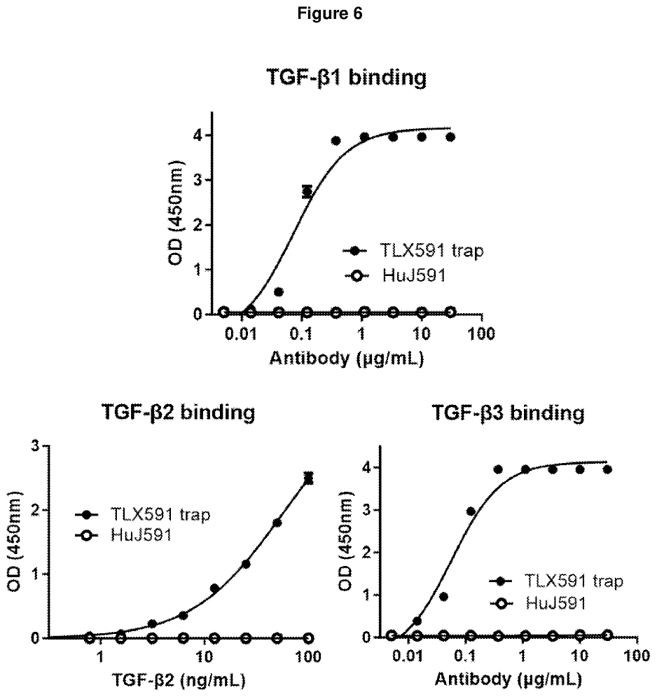

In vitro, TLX591 trap was shown to specifically bind to PSMA expressing LNCap cells in a dose dependent manner (See FIGS. 2 and 3). TLX591 trap was also shown to specifically bind to TGFβ1, TGFβ2 and TGFβ3 (FIG. 6).

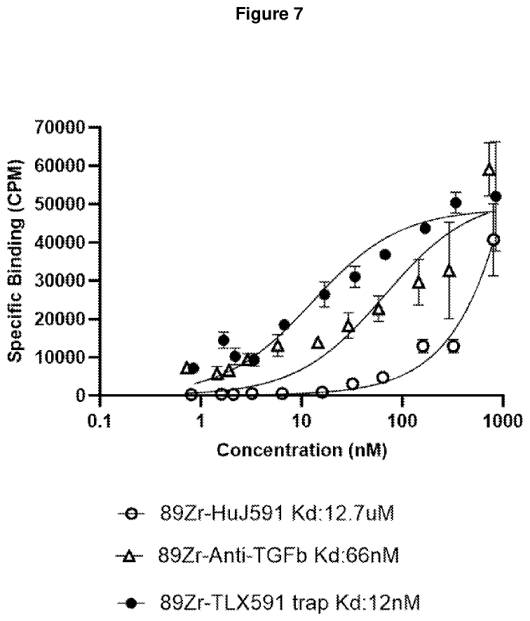

Further, ⁸⁹-Zr radiolabelled TLX591 trap has displayed high binding affinity to TGFβ1 in vitro (FIG. 7).

### Example 7: TLX591 Trap Sequesters TGFβ in the Tumour Microenvironment

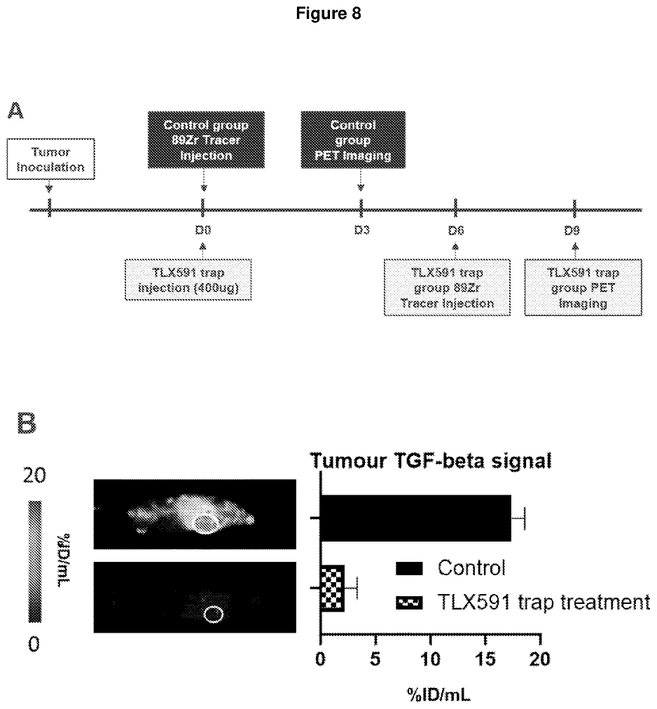

In an in vivo experiment, mice bearing RM1-hPSMA tumours were split into the following groups:
1) control group ⁸⁹-Zr Tracer alone (⁸⁹-Zr-labelled anti-TGFβ antibody, Fresolimumab)
2) TLX591 trap followed by ⁸⁹-Zr Tracer 6 days later.
   PET imaging was performed 3 days after administration of the ⁸⁹-Zr Tracer for both groups. The protocol timeline is shown in FIG. 8A.

As shown in FIG. 8B, administration of TLX591 trap was shown to significantly reduce detection of TGFβ in the tumour microenvironment compared to control.

### Example 8: TLX591 Trap Sequesters Circulating TGFβ Following External Beam Radiation Therapy (EBRT)

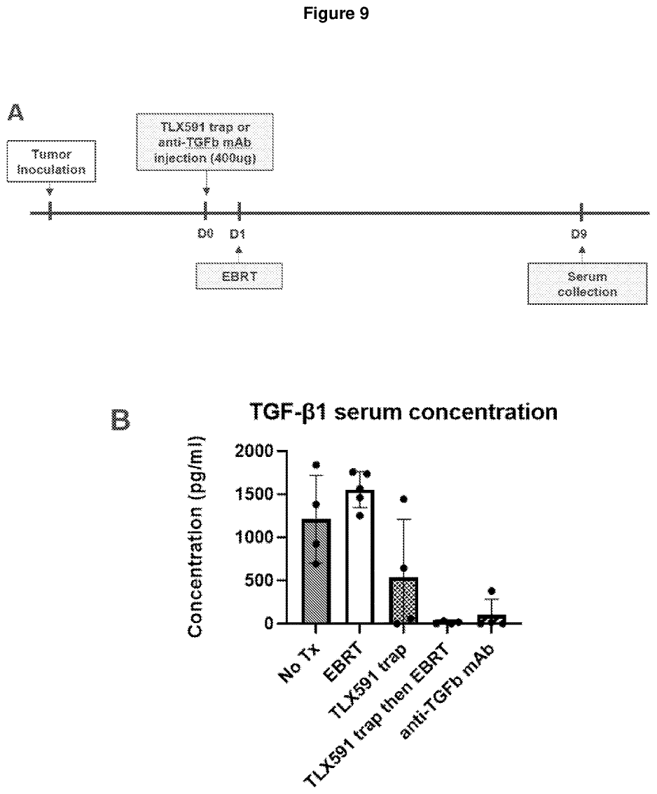

In another experiment, mice bearing RM1-hPSMA tumours were split into the following 5 groups:
1) no treatment on day 0;
2) EBRT treatment alone on day 1;
3) TLX591 trap alone on day 0;
4) TLX591 trap on day 0 followed by EBRT on day 1; and
5) anti-TGFβ mAb on day 0 followed by EBRT on day 1.
   Serum sample were collected on day 9 (see FIG. 9A for the protocol timeline).

As shown in FIG. 9B, administration of TLX591 trap prior to external beam radiation therapy (EBRT), significantly reduced TGFβ1 serum concentrations compared to no treatment prior to EBRT (FIG. 9B). The reduction in TGFβ1 serum concentrations was also comparable to treatment with control anti-TGFβ monoclonal antibody prior to EBRT (FIG. 9B).

It is expected that radiation-induced fibrosis in treatment group 4 will be lower compared to group 2.

### Example 9: Anti-CAIX-TGFβ Trap Administration Prior to External Beam Radiation (EBRT) to Reduce EBRT Side Effects

A molecule comprising an antigen binding domain for binding to CAIX and an ECD derived from TGFβR II is obtained by expressing in a mammalian cell line, a first expression construct (encoding a fusion protein comprising the sequence of SEQ ID NO: 255) and a second expression construct (encoding an antibody light chain as set forth in SEQ ID NO: 256). Upon expression of the proteins, the protein encoded by the first construct and the protein encoded by the second construct associate to form a single molecule through intramolecular disulphide bonding. This molecule is termed an anti-CAIX-TGFβ TRAP.

Standard in vitro assays of the molecule are conducted to confirm binding to CAIX (including CAIX-expressing tumour cells) and to TGFβ.

A mouse model is established in which tumours expressing human CAIX are engrafted into mice. Mice are split into 5 groups as follows:
1) no treatment;
2) EBRT treatment alone;
3) anti-CAIX TGFβ-trap trap alone;
4) anti-CAIX TGFβ-trap trap followed by EBRT; and
5) anti-TGFβ mAb followed by EBRT.

Mice are monitored for tumour growth. Some mice are sacrificed at fixed time points to monitor T cell infiltration and fibrosis.

The extent of fibrosis and T cell infiltration at the tumour site is assessed between treatment groups. The extent of radiation-induced fibrosis in treatment group 4 will be lower compared to group 2.

It will be understood that the invention disclosed and defined in this specification extends to all alternative combinations of two or more of the individual features mentioned or evident from the text or drawings. All of these different combinations constitute various alternative aspects of the invention.

1. A molecule comprising a tumour antigen binding protein and an extracellular domain (ECD) or ligand binding fragment of a transforming growth factor β receptor (TGFβR).

2. The molecule of claim 1, wherein the tumour antigen binding protein is selected from: an antigen binding domain of an immunoglobulin, antibody, bispecific or multispecific antibody, antibody fragment, single chain variable fragment (scFv), dimeric scFv, bivalent or multivalent scFv, a Fab, a F(ab′)2, an Fv, or a Fc-containing polypeptide.

3. The molecule of claim 1 or 2, wherein the tumour antigen binding protein is an antibody or antigen binding fragment thereof and the ECD or ligand binding fragment of a TGFβR is joined to the C terminus of the heavy chain of the antibody or fragment thereof.

4. The molecule of claim 1 or 2, wherein the tumour antigen binding protein is an antibody or antigen binding fragment thereof and the ECD or ligand binding fragment of a TGFβR is joined to the C terminus of the light chain of the antibody or fragment thereof.

5. The molecule of claim 1 or 2, wherein the tumour antigen binding protein is an antibody or antigen binding fragment thereof and the ECD or ligand binding fragment of a TGFβR is joined to a non-antigen binding portion of the antibody or antigen binding fragment thereof.

6. The molecule of any one of claims 1 to 5, wherein the tumour antigen binding protein binds to prostate specific membrane antigen (PSMA), carbonic anhydrase IX (CAIX), PDGFRα or La/SSB.

7. The molecule according to any one of claims 1 to 6, wherein the TGFβR is a type I (TGFβRI), type II (TGFβRII) or type III (TGFβRIII) TGFβR.

8. The molecule according to any one of claims 1 to 7, wherein the tumour antigen binding domain and the ECD or ligand binding fragment of a TGFβR are joined via a linker.

9. The molecule according to any one of claims 1 to 8, wherein the molecule is conjugated to a therapeutic agent, optionally via a linker or chelator moiety.

10. The molecule according to claim 9, wherein the therapeutic agent is a radioisotope.

11. The molecule according to claim 10, wherein the radioisotope is selected from the group consisting of: actinium-225 (225Ac), astatine-211 (211At), bismuth-212 and bismuth-213 (²¹²Bi, ²¹³Bi), copper-67 (⁶⁷Cu), iodine-123, -124, -125 or -131 (1231, 1241, 1251, 1311) (123 I), lead-212 (²¹²Pb), lutetium-177 (¹⁷⁷Lu), radium-223 and radium-224 (²²³Ra, ²²⁴Ra), samarium-153 (¹⁵³Sm), scandium-47 (⁴⁷Sc), strontium-90 (⁹⁰Sr), and yttrium-90 (⁹⁰Y) optionally wherein the radionuclide conjugated to the molecule is lutetium-¹⁷⁷.

12. The molecule according to any one of claims 9 to 11, wherein the therapeutic agent is conjugated to the portion of the molecule that comprises the tumour antigen binding protein.

13. The molecule according to any one of claims 9 to 11, wherein the therapeutic agent is conjugated to a portion of the molecule that comprises the ECD or ligand binding fragment of a TGFβR.

14. The molecule according to any one of claims 1 to 13, wherein the tumour antigen binding protein binds specifically to CAIX and wherein the antigen binding protein comprises:
FR1-CDR1-FR2-CDR2-FR3-CDR3-FR4 and FR1a-CDR1a-FR2a-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions;
wherein the sequence of any of the complementarity determining regions have an amino acid sequence as described in Table 2.

15. The molecule according to claim 14, wherein FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

16. The molecule of claim 14 or 15, wherein the tumour antigen binding protein comprises an antigen binding domain that consists essentially of or consists of an amino acid sequence of SEQ ID NO: 52, 68, 84, 100 or 116 (VH) and/or a sequence set forth in SEQ ID NO: 132, 148, 164, 180, 196 or 212 (VL).

17. The molecule of claim 14 or 15, wherein the tumour antigen binding protein comprises an antigen binding domain that comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO: 49, a CDR2 comprising a sequence at least about 80%, at least 85%, at least 90%, at least 92%, at least 95%, at least 97%, at least 99% identical to a sequence set in SEQ ID NO:50, and a CDR3 comprising a sequence at least about 80%, at least 85%, at least 90%, at least 92%, at least 95%, at least 97%, at least 99% identical to a sequence set forth in SEQ ID NO: 51;
(ii) a VH comprising a sequence at least about 95% or 96% or 97% or 98% or 99% identical to a sequence set forth in SEQ ID NO: 52, 68, 84, 100 or 116;
(iii) a VL comprising a CDR1 comprising a sequence at least about 80%, at least 85%, at least 90%, at least 92%, at least 95%, at least 97%, at least 99% identical to a sequence set forth in SEQ ID NO: 129, a CDR2 comprising a sequence at least about 80%, at least 85%, at least 90%, at least 92%, at least 95%, at least 97%, at least 99% identical to a sequence set forth in SEQ ID NO: 130, and a CDR3 comprising a sequence at least about 80%, at least 85%, at least 90%, at least 92%, at least 95%, at least 97%, at least 99% identical to a sequence set forth in SEQ ID NO: 131;
(iv) a VL comprising a sequence at least about 95% identical to a sequence set forth in SEQ ID NO: 132, 148, 164, 180, 196 or 212;
(v) a VH comprising a CDR1 comprising a sequence set forth in SEQ ID NO: 49, a CDR2 comprising a sequence set forth between in SEQ ID NO: 50, and a CDR3 comprising a sequence set forth in SEQ ID NO: 51;
(vi) a VH comprising a sequence set forth in SEQ ID NO: 52, 68, 84, 100 or 116;
(vii) a VL comprising a CDR1 comprising a sequence set SEQ ID NO: 129, a CDR2 comprising a sequence set forth in SEQ ID NO: 130, and a CDR3 comprising a sequence set forth in SEQ ID NO: 131;
(viii) a VL comprising a sequence set forth in SEQ ID NO: 132, 148, 164, 180, 196 or 212;
(ix) a VH comprising a CDR1 comprising a sequence set forth in SEQ ID NO: 49, a CDR2 comprising a sequence set forth between in SEQ ID NO: 50, and a CDR3 comprising a sequence set forth in SEQ ID NO: 51; and a VL comprising a CDR1 comprising a sequence set SEQ ID NO: 129, a CDR2 comprising a sequence set forth in SEQ ID NO: 130, and a CDR3 comprising a sequence set forth in SEQ ID NO: 131; or
(x) a VH comprising a sequence set forth in SEQ ID NO: 52, 68, 84, 100 or 116 and a VL comprising a sequence set forth in SEQ ID NO: 132, 148, 164, 180, 196 or 212.

18. The molecule of any one of claim 14, 15 or 17, wherein the tumour antigen binding protein comprises a variable heavy chain comprising an amino acid sequence as set forth in SEQ ID NO: 52, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX; and/or a variable light chain comprising an amino acid sequence as set forth in SEQ ID NO: 132, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for CAIX.

19. The molecule of claim 18, wherein the tumour antigen binding protein comprises the same CDR sequences as defined for the variable heavy chain of SEQ ID NO: 52 and/or the same CDR sequences as defined for the variable light chain of SEQ ID NO: 132.

20. The molecule according to any one of claims 1 to 13, wherein the tumour antigen binding protein binds specifically to PSMA and wherein the antigen binding protein comprises:
FR1-CDR1-FR2-CDR2-FR3-CDR3-FR4 and FR1a-CDR1a-FR2α-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
wherein the sequence of any of the complementarity determining regions have an amino acid sequence as described in Table 1.

21. The molecule according to claim 20, wherein FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

22. The molecule according to claim 20 or 21 wherein the tumour antigen binding protein comprises an antigen binding domain that consists essentially of or consists of an amino acid sequence of (in order of N to C terminus or C to N terminus) SEQ ID NO: 4, 20 or 244 (VH); and 36 or 245 (VL)

23. The molecule according to claim 20 or 21, wherein the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 1, a CDR2 comprising a sequence at least about 80%, at least 85%, at least 90%, at least 92%, at least 95%, at least 97%, at least 99% identical to a sequence set in SEQ ID NO: 2, and a CDR3 comprising a sequence at least about 80%, at least 85%, at least 90%, at least 92%, at least 95%, at least 97%, at least 99% identical to a sequence set forth in SEQ ID NO: 3;
(ii) a VH comprising a sequence at least about 95% or 96% or 97% or 98% or 99% identical to a sequence set forth in SEQ ID NO: 4, 20 or 244;
(iii) a VL comprising a CDR1 comprising a sequence at least about 80%, at least 85%, at least 90%, at least 92%, at least 95%, at least 97%, at least 99% identical to a sequence set forth in SEQ ID NO: 33, a CDR2 comprising a sequence at least about 80%, at least 85%, at least 90%, at least 92%, at least 95%, at least 97%, at least 99% identical to a sequence set forth in SEQ ID NO: 34 and a CDR3 comprising a sequence at least about 80%, at least 85%, at least 90%, at least 92%, at least 95%, at least 97%, at least 99% identical to a sequence set forth in SEQ ID NO: 35;
(iv) a VL comprising a sequence at least about 95% identical to a sequence set forth in SEQ ID NO: 36 or 245;
(v) a VH comprising a CDR1 comprising a sequence set forth in SEQ ID NO: 1, a CDR2 comprising a sequence set forth between in SEQ ID NO: 2, and a CDR3 comprising a sequence set forth in SEQ ID NO: 3;
(vi) a VH comprising a sequence set forth in SEQ ID NO: 4, 20 or 244;
(vii) a VL comprising a CDR1 comprising a sequence set SEQ ID NO: 33, a CDR2 comprising a sequence set forth in SEQ ID NO: 34 and a CDR3 comprising a sequence set forth in SEQ ID NO: 45;
(viii) a VL comprising a sequence set forth in SEQ ID NO: 36 or 245;
(ix) a VH comprising a CDR1 comprising a sequence set forth in SEQ ID NO: 1, a CDR2 comprising a sequence set forth between in SEQ ID NO: 2 and a CDR3 comprising a sequence set forth in SEQ ID NO: 3; and a VL comprising a CDR1 comprising a sequence set SEQ ID NO: 33, a CDR2 comprising a sequence set forth in SEQ ID NO: 34 and a CDR3 comprising a sequence set forth in SEQ ID NO: 35; or
(x) a VH comprising a sequence set forth in SEQ ID NO: 4, 20 or 244 and a VL comprising a sequence set forth in SEQ ID NO: 36 or 245.

24. The molecule according to claim 20, 21 or 23, wherein the tumour antigen binding protein comprises a variable heavy chain comprising an amino acid sequence as set forth in SEQ ID NO: 244, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PSMA; and/or a variable light chain comprising an amino acid sequence as set forth in SEQ ID NO: 245, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PSMA.

25. The molecule according to claim 24, wherein the tumour antigen binding protein comprises the same CDR sequences as defined for the variable heavy chain of SEQ ID NO: 244 and/or the same CDR sequences as defined for the variable light chain of SEQ ID NO: 245.

26. The molecule according to any one of claims 1 to 13, wherein the tumour antigen binding protein binds specifically to PDGFRα and wherein the antigen binding protein comprises:
FR1-CDR1-FR2-CDR2-FR3-CDR3-FR4 and FR1a-CDR1a-FR2α-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
wherein the sequence of any of the complementarity determining regions have an amino acid sequence as described in Table 5.

27. The molecule according to claim 26, wherein FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

28. The molecule according to claim 26 or 27 wherein the tumour antigen binding protein comprises an antigen binding domain that consists essentially of or consists of an amino acid sequence of (in order of N to C terminus or C to N terminus) SEQ ID NO: 265 and/or 266.

29. The molecule according to claim 26 or 27, wherein the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 267, a CDR2 comprising a sequence at least about 80%, at least 85%, at least 90%, at least 92%, at least 95%, at least 97%, at least 99% identical to a sequence set in SEQ ID NO: 268, and a CDR3 comprising a sequence at least about 80%, at least 85%, at least 90%, at least 92%, at least 95%, at least 97%, at least 99% identical to a sequence set forth in SEQ ID NO: 269;
(ii) a VH comprising a sequence at least about 95% or 96% or 97% or 98% or 99% identical to a sequence set forth in SEQ ID NO: 265;
(iii) a VL comprising a CDR1 comprising a sequence at least about 80%, at least 85%, at least 90%, at least 92%, at least 95%, at least 97%, at least 99% identical to a sequence set forth in SEQ ID NO: 270, a CDR2 comprising a sequence at least about 80%, at least 85%, at least 90%, at least 92%, at least 95%, at least 97%, at least 99% identical to a sequence set forth in SEQ ID NO: 271 and a CDR3 comprising a sequence at least about 80%, at least 85%, at least 90%, at least 92%, at least 95%, at least 97%, at least 99% identical to a sequence set forth in SEQ ID NO: 272;
(iv) a VL comprising a sequence at least about 95% identical to a sequence set forth in SEQ ID NO:266;
(v) a VH comprising a CDR1 comprising a sequence set forth in SEQ ID NO: 267, a CDR2 comprising a sequence set forth between in SEQ ID NO: 268 and a CDR3 comprising a sequence set forth in SEQ ID NO: 269;
(vi) a VH comprising a sequence set forth in SEQ ID NO: 265;
(vii) a VL comprising a CDR1 comprising a sequence set SEQ ID NO: 270, a CDR2 comprising a sequence set forth in SEQ ID NO: 271 and a CDR3 comprising a sequence set forth in SEQ ID NO: 272;
(viii) a VL comprising a sequence set forth in SEQ ID NO: 266;
(ix) a VH comprising a CDR1 comprising a sequence set forth in SEQ ID NO: 267, a CDR2 comprising a sequence set forth between in SEQ ID NO: 268 and a CDR3 comprising a sequence set forth in SEQ ID NO: 269; and a VL comprising a CDR1 comprising a sequence set SEQ ID NO: 270, a CDR2 comprising a sequence set forth in SEQ ID NO: 271 and a CDR3 comprising a sequence set forth in SEQ ID NO: 272; or
(x) a VH comprising a sequence set forth in SEQ ID NO: 265 and a VL comprising a sequence set forth in SEQ ID NO: 266.

30. The molecule according to claim 26, 27 or 29, wherein the tumour antigen binding protein comprises a variable heavy chain comprising an amino acid sequence as set forth in SEQ ID NO: 265, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PDGFRα; and/or a variable light chain comprising an amino acid sequence as set forth in SEQ ID NO: 266, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for PDGFRα.

31. The molecule according to claim 30, wherein the tumour antigen binding protein comprises the same CDR sequences as defined for the variable heavy chain of SEQ ID NO: 265 and/or the same CDR sequences as defined for the variable light chain of SEQ ID NO: 266.

32. The molecule according to any one of claims 1 to 13, wherein the tumour antigen binding protein binds specifically to La/SSB and wherein the antigen binding protein comprises:
FR1-CDR1-FR2-CDR2-FR3-CDR3-FR4 and FR1a-CDR1a-FR2α-CDR2a-FR3a-CDR3a-FR4a
wherein:
FR1, FR2, FR3 and FR4 are each framework regions;
CDR1, CDR2 and CDR3 are each complementarity determining regions;
FR1a, FR2a, FR3a and FR4a are each framework regions;
CDR1a, CDR2a and CDR3a are each complementarity determining regions; and
wherein the sequence of any of the complementarity determining regions have an amino acid sequence as described in Table 6.

33. The molecule according to claim 26, wherein FR1-CDR1-FR2-CDR2-FR3-CDR3-FR4 and FR1a-CDR1a-FR2a-CDR2a-FR3a-CDR3a-FR4a are linked via a linker, optionally in the form of a chemical, one or more amino acids, or a disulphide bond formed between two cysteine residues.

34. The molecule according to claim 32 or 33 wherein the tumour antigen binding protein comprises an antigen binding domain that consists essentially of or consists of an amino acid sequence of (in order of N to C terminus or C to N terminus) SEQ ID NO: 303 and/or 304.

35. The molecule according to claim 33 or 33, wherein the tumour antigen binding protein comprises at least one of:
(i) a VH comprising a complementarity determining region (CDR) 1 comprising a sequence at least about 80%, at least 85%, at least 90%, at least 92%, at least 95%, at least 97%, at least 99% identical to a sequence set forth in SEQ ID NO 305, a CDR2 comprising a sequence at least about 80%, at least 85%, at least 90%, at least 92%, at least 95%, at least 97%, at least 99% identical to a sequence set in SEQ ID NO: 306, and a CDR3 comprising a sequence at least about 80%, at least 85%, at least 90%, at least 92%, at least 95%, at least 97%, at least 99% identical to a sequence set forth in SEQ ID NO: 307;
(ii) a VH comprising a sequence at least about 95% or 96% or 97% or 98% or 99% identical to a sequence set forth in SEQ ID NO: 303;
(iii) a VL comprising a CDR1 comprising a sequence at least about 80%, at least 85%, at least 90%, at least 92%, at least 95%, at least 97%, at least 99% identical to a sequence set forth in SEQ ID NO: 308, a CDR2 comprising a sequence at least about 80%, at least 85%, at least 90%, at least 92%, at least 95%, at least 97%, at least 99% identical to a sequence set forth in SEQ ID NO: 309 and a CDR3 comprising a sequence at least about 80%, at least 85%, at least 90%, at least 92%, at least 95%, at least 97%, at least 99% identical to a sequence set forth in SEQ ID NO: 310;
(iv) a VL comprising a sequence at least about 95% identical to a sequence set forth in SEQ ID NO: 304;
(v) a VH comprising a CDR1 comprising a sequence set forth in SEQ ID NO: 305, a CDR2 comprising a sequence set forth between in SEQ ID NO: 306 and a CDR3 comprising a sequence set forth in SEQ ID NO: 307;
(vi) a VH comprising a sequence set forth in SEQ ID NO: 303;
(vii) a VL comprising a CDR1 comprising a sequence set SEQ ID NO: 308, a CDR2 comprising a sequence set forth in SEQ ID NO: 309 and a CDR3 comprising a sequence set forth in SEQ ID NO: 310;
(viii) a VL comprising a sequence set forth in SEQ ID NO: 304;
(ix) a VH comprising a CDR1 comprising a sequence set forth in SEQ ID NO: 305, a CDR2 comprising a sequence set forth between in SEQ ID NO: 306 and a CDR3 comprising a sequence set forth in SEQ ID NO: 307; and a VL comprising a CDR1 comprising a sequence set SEQ ID NO: 308, a CDR2 comprising a sequence set forth in SEQ ID NO: 309 and a CDR3 comprising a sequence set forth in SEQ ID NO: 310; or
(x) a VH comprising a sequence set forth in SEQ ID NO: 303 and a VL comprising a sequence set forth in SEQ ID NO: 304.

36. The molecule according to claim 33, 34 or 35, wherein the tumour antigen binding protein comprises a variable heavy chain comprising an amino acid sequence as set forth in SEQ ID NO: 303, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for La/SSB; and/or a variable light chain comprising an amino acid sequence as set forth in SEQ ID NO: 304, or a sequence that is at least 80%, 81%, 82%, 84%, 84%, 85%, 86%, 87%, 88%, 89%, 90%, 91%, 92%, 93%, 94%, 95% or 96% or 97% or 98% or 99% identical thereto, while retaining binding affinity for La/SSB.

37. The molecule according to claim 36, wherein the tumour antigen binding protein comprises the same CDR sequences as defined for the variable heavy chain of SEQ ID NO: 303 and/or the same CDR sequences as defined for the variable light chain of SEQ ID NO: 304.

38. The molecule according to any one of claims 1 to 37, wherein the antigen binding protein comprises a human constant region.

39. The molecule according to claim 38, wherein the heavy chain constant region comprises one or more amino acid substitutions for stabilising the linkage to the ECD or ligand binding domain of the TGFβR and/or one or more amino acid substitutions for reducing the serum half-life of the molecule, as herein described.

40. The molecule according to any one of claims 1 to 39, wherein the ECD or ligand binding domain of the TGFβR comprises or consists of an amino acid sequence as set forth in any one of SEQ ID NOs: 246 to 254, or as described in Table 4.

41. The molecule according to any one of claims 1 to 40, wherein the ECD or ligand binding domain of the TGFβR comprises or consists of the amino acid sequence as set forth in SEQ ID NO: 249 or 321 or a sequence at least about 80%, 81%, 82%, 84%, 84%, 85%, 86%, 87%, 88%, 89%, 90%, 91%, 92%, 93%, 94%, 95% or 96% or 97% or 98% or 99% identical thereto, which retains the ability to bind to TGFβ.

42. The molecule according to any one of claims 1 to 19, wherein the molecule comprises an antibody for binding CAIX and comprises a heavy chain comprising the amino acid sequence as set forth in SEQ ID NO: 257 or 258 and a light chain comprising the amino acid sequence as set forth in SEQ ID NO: 259.

43. The molecule according to any one of claims 1 to 13 and 20 to 25, wherein the molecule comprises an antibody for binding PSMA and comprises a heavy chain comprising the amino acid sequence as set forth in SEQ ID NO: 262 or 263 and a light chain comprising the amino acid sequence as set forth in SEQ ID NO: 264.

44. A therapeutic IgG molecule comprising:
heavy and light chains, wherein each chain comprises a variable region and a constant region, and at least one variable region binds a tumour antigen;
a radioisotope conjugated to one or more amino acid residues of the heavy or light chains; and
an extracellular domain (ECD) or ligand binding fragment of a transforming growth factor β receptor (TGFβR).

45. The therapeutic IgG molecule according to claim 44, wherein the variable region of the heavy chain comprises an amino acid sequence as set forth in any one of SEQ ID NO: 52, 68, 84, 100, and 116.

46. The therapeutic IgG molecule according to any one of claim 44 or 45, wherein the variable region of the light chain comprises an amino acid sequence as set forth in any one of: SEQ ID NOs: 132, 148, 164, 180, 196 and 212.

47. The therapeutic IgG molecule according to claim 44, wherein the variable region of the heavy chain comprises an amino acid sequence as set forth in SEQ ID NO: 4, 20 or 244.

48. The therapeutic IgG molecule according to claim 44 or 47, wherein the variable region of the light chain comprises an amino acid sequence as set forth in SEQ ID NO: 36 or 245.

49. The therapeutic IgG molecule according to claim 44, wherein the variable region of the heavy chain comprises an amino acid sequence as set forth in SEQ ID NO: 265.

50. The therapeutic IgG molecule according to claim 44 or 49, wherein the variable region of the light chain comprises an amino acid sequence as set forth in SEQ ID NO: 266.

51. The therapeutic IgG molecule according to claim 44, wherein the variable region of the heavy chain comprises an amino acid sequence as set forth in SEQ ID NO: 303.

52. The therapeutic IgG molecule according to claim 44 or 51, wherein the variable region of the light chain comprises an amino acid sequence as set forth in SEQ ID NO: 304.

53. The therapeutic IgG molecule according to any one of claims 44 to 52, wherein heavy chain constant region of the molecule comprises one or more amino acid substitutions for altering the serum half-life of the molecule, optionally wherein the substitutions are at position His310 or at His435, preferably, wherein the amino acid substitutions are at both His310 and His435, or positions equivalent thereto.

54. The therapeutic IgG molecule according to any one of claims 44 to 53, wherein the molecule further comprises: (a) one or more amino acid substitutions that reduce the affinity of the molecule for one or more Fc gamma receptors compared to a wild-type antibody of the class IgG; and/or (b) one or more amino acid substitutions that increase the stability of the CH1-CH2 hinge region in the molecule compared to a wild-type antibody of the class IgG.

55. The therapeutic IgG molecule according to claim 54, wherein the one or more amino acid substitutions that reduce affinity of the molecule for an Fc gamma receptor comprises a substitution at position Leu235, or position equivalent thereto.

56. The therapeutic IgG molecule according to claim 54 or 55 wherein the one or more amino acid substitutions that increase the stability of the CH1-CH2 hinge region in the molecule comprises a substitution at IgG molecule.

57. The therapeutic antibody according to any one of claims 55 to 56 wherein the heavy chain comprises an amino acid sequence as set forth in SEQ ID NO: 231.

58. The therapeutic IgG molecule according to any one of claims 44 to 57 wherein the light chain comprises an amino acid sequence as set forth in SEQ ID NO: 234.

59. The therapeutic antibody of any one of claims 44 to 58 wherein the heavy chain comprises an amino acid sequence as set forth in SEQ ID NO: 53.

60. The therapeutic IgG molecule of any one of claims 44 to 59 wherein the light chain comprises an amino acid sequence as set forth in SEQ ID NO: 57.

61. The therapeutic IgG molecule of any one of claims 44 to 60 wherein the antibody comprises the amino acid sequence as set forth in SEQ ID NO: 53 and the antibody comprises the amino acid sequence set forth in SEQ ID NO: 57.

62. The therapeutic IgG molecule according to any one of claims 44 to 61 wherein the radioisotope is selected from the group consisting of: actinium-225 (225Ac), astatine-211 (211At), bismuth-212 and bismuth-213 (²¹²Bi, ²¹³Bi), copper-67 (⁶⁷Cu), iodine-123, -124, -125 or -131 (¹²³I, ¹²⁴I, ¹²⁵I, ¹³¹I) (¹²³I), lead-212 (²¹²Pb), lutetium-177 (¹⁷⁷Lu), radium-223 and radium-224 (²²³Ra, ²²⁴Ra), samarium-153 (¹⁵³Sm), scandium-47 (⁴⁷Sc), strontium-90 (⁹⁰Sr), and yttrium-90 (⁹⁰Y).

63. The therapeutic IgG molecule according to any one of claims 44 to 62 wherein the radioactive element is selected from: actinium-225 (²²⁵Ac), astatine-211 (²¹¹At), and lutetium-177 (¹⁷⁷Lu).

64. The therapeutic IgG molecule according to any one of claims 44 to 63, wherein the ECD or ligand binding domain of the TGFβR comprises or consists of an amino acid sequence derived from a TGFβRII.

65. The therapeutic IgG molecule according to any one of claims 44 to 64, wherein the ECD or ligand binding domain of the TGFβR comprises or consists of an amino acid sequence as set forth in any one of SEQ ID NOs: 246 to 254, or as described in Table 4.

66. A composition comprising a molecule according to of any one of claims 1 to 43, or a therapeutic IgG molecule of any one of claims 44 to 65, and a pharmaceutically acceptable excipient or carrier.

67. A bioconjugate comprising a molecule according to claims 1 to 8 conjugated to a chelator moiety or linker group.

68. The bioconjugate according to claim 67, wherein the chelator moiety is selected from TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-NN′,N″(N′″-tetraacetic acid), TCMC, DO3A, CB-DO2A, NOTA, Diamsar, DTPA, CHX-A″-DTPA, TETE, Te2A, HBED, DFO, DFOsq and HOPO.

69. A method of treating cancer in an individual, the method comprising administering to an individual in need thereof, a molecule of any one of claims 1 to 43, or a therapeutic IgG molecule of any one of claims 44 to 65, or a composition of claim 66.

70. Use of a molecule of any one of claims 1 to 43, or a therapeutic IgG molecule of any one of claims 44 to 65, or a bioconjugate according to claim 67 or 68, in the manufacture of a medicament for the treatment of cancer in a subject.

71. A molecule of any one of claims 1 to 43, or a therapeutic IgG of any one of claims 44 to 65, or the composition of claim 66, for use in the treatment of cancer in a subject.

72. A method of treating cancer in an individual, the method comprising administering to a subject in need thereof, (a) a molecule of any one of claims 1 to 43, or a therapeutic IgG molecule of any one of claims 44 to 54, or a composition of claim 66, and (b) a molecule comprising an immunoglobulin moiety and a non-protein agent conjugated thereto, wherein the immunoglobulin moiety specifically binds to a tumour antigen, wherein the non-protein agent comprises a therapeutic moiety, preferably a cytotoxin or a radioactive element.

73. A method of claim 72, wherein the molecule or therapeutic molecule of (a) does not comprise a radionuclide.

74. A method of any one of claim 69 or 72 to 73, or the use of claim 70, or the molecule or therapeutic IgG for use of claim 71, wherein the subject has received a prior treatment for cancer that is suspected of causing, or causes, an increase in TGFβ activity in the tumour microenvironment.

75. The method or use of claim 74, wherein the prior treatment for cancer is selected from the group consisting of: treatment with external beam radiation (EBR), treatment with a chemotherapeutic agent, surgery or resection of the tumour, treatment with an immunomodulatory agent, including a CPI, treatment with a molecular targeted radionuclide (MTR), treatment with a cell therapy, such as CAR T therapy.

76. A method of reducing or inhibiting radiation-induced TGFβ activity in a subject receiving or requiring radiation treatment for cancer, wherein the method comprises administering a molecule of any one of claims 1 to 43, or a therapeutic IgG molecule of any one of claims 44 to 65, or a composition of claim 66.

77. The method or use of claim 76, wherein the method is for reducing or inhibiting radiation-induced fibrosis in the subject.

78. A method for enhancing or increasing the likelihood of success of treatment with an immune checkpoint inhibitor in a subject, wherein the method comprises administering to a subject requiring treatment with an immune checkpoint inhibitor, a molecule of any one of claims 1 to 43, or a therapeutic IgG molecule of any one of claims 44 to 65, or a composition of claim 66.

79. The method or use of claim 78, wherein the subject has received or is receiving treatment with external beam radiation, or molecular targeted radiation, or any other treatment which increases the activity of TGFβ in the tumour microenvironment.

80. A method of any one of claim 69 or 72 to 73, or the use of claim 70, or the molecule or therapeutic IgG for use of claim 71, wherein the method comprises concomitantly or subsequently administering a treatment for cancer selected from the group consisting of: treatment with external beam radiation (EBR), treatment with a chemotherapeutic agent, surgery or resection of the tumour, treatment with an immunomodulatory agent, including an immune checkpoint inhibitor, treatment with a molecular targeted radionuclide (MTR), treatment with a cell therapy, such as CAR T therapy.

81. A method of reducing or inhibiting radiation-induced TGFβ activity in an individual comprising:
administering to the individual, a molecule of any one of claims 1 to 43, or a therapeutic IgG molecule of any one of claims 44 to 65, or a composition of claim 66; and
administering to the subject a treatment for cancer that is suspected or known to cause an increase in TGFβ activity in the tumour microenvironment, thereby reducing or inhibiting radiation-induced TGFβ activity in the subject.

82. The method of claim 81, wherein the treatment for cancer is a molecular targeted radionuclide (MTR).

83. The method of claim 82, wherein the MTR comprises a radiolabelled antigen binding protein for binding to CAIX.

84. The method of claim 82, wherein the MTR comprises a radiolabelled antigen binding protein for binding to PSMA.

85. The method of claim 82, wherein the MTR comprises a radiolabelled antigen binding protein for binding to PDGFRα.

86. The method of claim 82, wherein the MTR comprises a radiolabelled antigen binding protein for binding to La/SSB.
